SEQUENCE LISTING (83 sequences)

SEQ ID NO: 1 - protein, 152 aa, artificial
  IL7
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 152 aa, artificial
  IL7-SS1
DSDIEGKDGKQYESVLMVSIDQLLDSMKEIGSNSLNNEFNFFKRHICDANKEGMFLFRAARKLRQFLKMNSTGDFDLHLLKVSEGTTILLNCTGQVKGRKPAALGEAQPTKSLEENKSLKEQKKLNDLSFLKRLLQEIKTSWNKILMGTKEH

SEQ ID NO: 3 - protein, 152 aa, artificial
  IL7-SS2
DSDIEGKDGKQYESVLMVSIDQLLDSMKEIGSNCLNNEFNFFKRHISDANKEGMFLFRAARKLRQFLKMNSTGDFDLHLLKVSEGTTILLNSTGQVKGRKPAALGEAQPTKSLEENKSLKEQKKLNDLCFLKRLLQEIKTSWNKILMGTKEH

SEQ ID NO: 4 - protein, 152 aa, artificial
  IL7-SS3
DCDIEGKDGKQYESVLMVSIDQLLDSMKEIGSNSLNNEFNFFKRHISDANKEGMFLFRAARKLRQFLKMNSTGDFDLHLLKVSEGTTILLNSTGQVKGRKPAALGEAQPTKSLEENKSLKEQKKLNDLSFLKRLLQEIKTCWNKILMGTKEH

SEQ ID NO: 5 - protein, 152 aa, artificial
  IL7-W142H
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 6 - protein, 152 aa, artificial
  IL7-W142Y
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCYNKILMGTKEH

SEQ ID NO: 7 - protein, 152 aa, artificial
  IL7-W142F
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCFNKILMGTKEH

SEQ ID NO: 8 - protein, 152 aa, artificial
  IL7-Q11E
DCDIEGKDGKEYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 9 - protein, 152 aa, artificial
  IL7-Y12F
DCDIEGKDGKQFESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 10 - protein, 152 aa, artificial
  IL7-M17L
DCDIEGKDGKQYESVLLVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 11 - protein, 152 aa, artificial
  IL7-Q22E
DCDIEGKDGKQYESVLMVSIDELLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 12 - protein, 152 aa, artificial
  IL7-D74E
DCDIEGKDGKQYESVLMVSIDQLLDSMKEIGSNCLNNEFNFFKRHICDANKEGMFLFRAARKLRQFLKMNSTGEFDLHLLKVSEGTTILLNCTGQVKGRKPAALGEAQPTKSLEENKSLKEQKKLNDLCFLKRLLQEIKTCWNKILMGTKEH

SEQ ID NO: 13 - protein, 152 aa, Artificial Sequence
  IL7-D74Q
DCDIEGKDGKQYESVLMVSIDQLLDSMKEIGSNCLNNEFNFFKRHICDANKEGMFLFRAARKLRQFLKMNSTGQFDLHLLKVSEGTTILLNCTGQVKGRKPAALGEAQPTKSLEENKSLKEQKKLNDLCFLKRLLQEIKTCWNKILMGTKEH

SEQ ID NO: 14 - protein, 152 aa, artificial
  IL7-D74N
DCDIEGKDGKQYESVLMVSIDQLLDSMKEIGSNCLNNEFNFFKRHICDANKEGMFLFRAARKLRQFLKMNSTGNFDLHLLKVSEGTTILLNCTGQVKGRKPAALGEAQPTKSLEENKSLKEQKKLNDLCFLKRLLQEIKTCWNKILMGTKEH

SEQ ID NO: 15 - protein, 152 aa, artificial
  IL7-K81R
DCDIEGKDGKQYESVLMVSIDQLLDSMKEIGSNCLNNEFNFFKRHICDANKEGMFLFRAARKLRQFLKMNSTGDFDLHLLRVSEGTTILLNCTGQVKGRKPAALGEAQPTKSLEENKSLKEQKKLNDLCFLKRLLQEIKTCWNKILMGTKEH

SEQ ID NO: 16 - protein, 9 aa, artificial
  LC CDR3
FQGTHVPNT

SEQ ID NO: 17 - protein, 117 aa, artificial
  HC variable domain
  MISC_FEATURE (105)..(105): X is X1, D or E
  MISC_FEATURE (106)..(106): X is X2, and is T, H, A, Y, N, E or S
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMXXWGQGTLVTVSS

SEQ ID NO: 18 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSS

SEQ ID NO: 19 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSS

SEQ ID NO: 20 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSS

SEQ ID NO: 21 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSS

SEQ ID NO: 22 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSS

SEQ ID NO: 23 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSS

SEQ ID NO: 24 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSS

SEQ ID NO: 25 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSS

SEQ ID NO: 26 - protein, 112 aa, artificial
  LC variable domain
  MISC_FEATURE (34)..(34): X is G or T
DVVMTQSPLSLPVTLGQPASISCRSSQSLVHANXNTYLEWYQQRPGQSPRLLIYKVSNRFSGVPDRFSGSGSGTDFTLKISRVEAEDVGVYYCFQGTHVPNTFGQGTKLEIK

SEQ ID NO: 27 - protein, 112 aa, artificial
  LC variable domain
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIK

SEQ ID NO: 28 - protein, 112 aa, artificial
  LC variable domain
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIK

SEQ ID NO: 29 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 30 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 32 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 33 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 34 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 35 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 36 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 37 - protein, 219 aa, artificial
  LC
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 219 aa, artificial
  LC
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 327 aa, artificial
  HC constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 40 - protein, 107 aa, artificial [sequence omitted]

SEQ ID NO: 41 - protein, 30 aa, artificial
  HFR1
QIQLVQSGSELKKPGASVKVSCKASGYTFT

SEQ ID NO: 42 - protein, 14 aa, artificial
  HFR2
WVRQAPGQGLEWMG

SEQ ID NO: 43 - protein, 32 aa, artificial
  HFR3
RFVFSLDTSVSTAYLQISSLKAEDTAVYYCAR

SEQ ID NO: 44 - protein, 11 aa, artificial
  HFR4
WGQGTLVTVSS

SEQ ID NO: 45 - protein, 23 aa, artificial
  LFR1
DVVMTQSPLSLPVTLGQPASISC

SEQ ID NO: 46 - protein, 15 aa, artificial
  LFR2
WYQQRPGQSPRLLIY

SEQ ID NO: 47 - protein, 32 aa, artificial
  LFR3
GVPDRFSGSGSGTDFTLKISRVEAEDVGVYYC

SEQ ID NO: 48 - protein, 10 aa, artificial
  LFR4
FGQGTKLEIK

SEQ ID NO: 49 - protein, 19 aa, artificial
  signal peptide
MDWLWNLLFLMAAAQSIQA

SEQ ID NO: 50 - protein, 19 aa, artificial
  signal peptide
MKLPVRLLVLMFWIPASSS

SEQ ID NO: 51 - protein, 5 aa, artificial
  HC CDR1
HYAMN

SEQ ID NO: 52 - protein, 330 aa, artificial
  Heavy chain constant domain (IgG1m-N298A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 17 aa, artificial
  HC CDR2
WINTNTGEPTYAQGFTG

SEQ ID NO: 54 - protein, 8 aa, artificial
  HC CDR3
  MISC_FEATURE (7)..(7): X is X1 (D or E)
  MISC_FEATURE (8)..(8): X is X2 and is T, H, A, Y, N, E or S
EREPGMXX

SEQ ID NO: 55 - protein, 8 aa, artificial
  HC CDR3
EREPGMDS

SEQ ID NO: 56 - protein, 8 aa, artificial
  HC CDR3
EREPGMDT

SEQ ID NO: 57 - protein, 8 aa, artificial
  HC CDR3
EREPGMES

SEQ ID NO: 58 - protein, 8 aa, artificial
  HC CDR3
EREPGMDH

SEQ ID NO: 59 - protein, 8 aa, artificial
  HC CDR3
EREPGMDA

SEQ ID NO: 60 - protein, 8 aa, artificial
  HC CDR3
EREPGMDY

SEQ ID NO: 61 - protein, 8 aa, artificial
  HC CDR3
EREPGMDN

SEQ ID NO: 62 - protein, 8 aa, artificial
  HC CDR3
EREPGMDE

SEQ ID NO: 63 - protein, 16 aa, artificial
  LC CDR1
  MISC_FEATURE (11)..(11): X is G or T
RSSQSLVHANXNTYLE

SEQ ID NO: 64 - protein, 16 aa, artificial
  LC CDR1
RSSQSLVHANGNTYLE

SEQ ID NO: 65 - protein, 16 aa, artificial
  LC CDR1
RSSQSLVHANTNTYLE

SEQ ID NO: 66 - protein, 7 aa, artificial
  LC CDR2
KVSNRFS

SEQ ID NO: 67 - protein, 9 aa, artificial
  linker C
GGGGSGGGS

SEQ ID NO: 68 - protein, 5 aa, artificial
  linker G4S
GGGGS

SEQ ID NO: 69 - protein, 10 aa, artificial
  linker (G4S)2
GGGGSGGGGS

SEQ ID NO: 70 - protein, 15 aa, artificial
  linker (G4S)3
GGGGSGGGGSGGGGS

SEQ ID NO: 71 - protein, 444 aa, artificial
  HC anti-PD1 chimeric
QIQLVQSGPELKKPGETVKISCKASGYTFTHYGMNWVKQAPGKGLKWMGWINTNTGEPTYAEEFKGRFAFSLETSASAAFLQINNLKNEDTATYFCAKEREPGMDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 72 - protein, 219 aa, artificial
  LC anti-PD1 chimeric
DVLLTQTPLSLPVSLGDQASISCRSSQNIVHANGNTYLEWYLQKPGQSPKLLIYKVSNRFSGVPDRFSGSGSGTDFTLKITRVEAEDLGVYYCFQGSHVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 2670 bp, artificial
  VH-VK
cagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgacagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 74 - DNA, 660 bp, artificial
  VL-VD
gacgtggtcatgacacagagcccactgtctctgcctgtgaccctgggacagccagcctctatctcctgcagatccagccagtctctggtgcacgctaacaccaatacatacctggagtggtatcagcagaggccaggacagtccccaaggctgctgatctacaaggtgtccaacagattcagcggagtgccagaccgctttagcggatctggatccggaaccgacttcaccctgaagatctccagggtggaggctgaggatgtgggcgtgtactattgtttccagggcacccatgtgcctaatacatttggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 75 - protein, 227 aa, artificial
  KIH-FcG1m (Knob)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 394 aa, artificial
  KIH-FcG1m-(G4S)3-W142H (Knob)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 77 - protein, 227 aa, artificial
  KIH-FcG1m (Hole)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 78 - protein, 394 aa, artificial
  KIH-FcG1m-(G4S)3-W142H (Hole)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 79 - protein, 220 aa, artificial
  VHOSE279-CH1
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 80 - protein, 219 aa, artificial; identical to SEQ ID NO: 38
  VLOSE279-CL

SEQ ID NO: 81 - protein, 447 aa, artificial
  VHOSE279-CH1-KIH-FcG1m (Knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 614 aa, artificial
  VHOSE279-CH1-KIH-FcG1m-(G4S)3-W142H (Knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

SEQ ID NO: 83 - protein, 614 aa, artificial
  VHOSE279-CH1-KIH-FcG1m-(G4S)3-W142H (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCHNKILMGTKEH

(19) United States
(12) Patent Application Publication
(10) Pub. No.: US 2023/0303648 A1
(43) Pub. Date: Sep. 28, 2023

(54) BIFUNCTIONAL MOLECULES COMPRISING AN IL-7 VARIANT

(71) Applicant: OSE IMMUNOTHERAPEUTICS, NANTES (FR)

(72) Inventors: NICOLAS POIRIER, TREILLIERES (FR); CAROLINE MARY, SAINTE PAZANNE (FR); AURORE MORELLO, SAINT SEBASTIEN SUR LOIRE (FR)

(21) Appl. No.: 17/785,427

(22) PCT Filed: Dec. 17, 2020
(86) PCT No.: PCT/EP2020/086600

§ 371 Date: Jun. 15, 2022

(30) Foreign Application Priority Data
Dec. 17, 2019 (EP) 19306671.9

(51) Int. Cl. C07K 14/54; A61K 47/68; A61P 35/00

(52) U.S. Cl. CPC C07K 14/5418; A61K 47/6813; A61K 47/6851; A61P 35/00

(57) ABSTRACT

The present invention relates to IL-7 variants, bifunctional molecules comprising it and their uses.

### CROSS-REFERENCE TO RELATED APPLICATION

This application is the U.S. national stage application of International Patent Application No. PCT/EP2020/086600, filed Dec. 17, 2020.

### REFERENCE TO SEQUENCE LISTING

The Sequence Listing for this application is labeled “Seq-List.txt” which was created on Dec. 3, 2020 and is 122,578 bytes. The entire content of the sequence listing is incorporated herein by reference in its entirety.

### FIELD OF THE INVENTION

The invention pertains to the field of immunotherapy. The present invention provides a bifunctional molecule that comprises an IL-7 variant.

### BACKGROUND OF THE INVENTION

Interleukin-7 is an immunostimulatory cytokine member of the IL-2 superfamily and plays an important role in an adaptive immune system by promoting immune responses. This cytokine activates immune functions through the survival and differentiation of T cells and B cells, survival of lymphoid cells, stimulation of activity of natural killer (NK) cell. IL-7 also regulates the development of lymph nodes through lymphoid tissue inducer (LTi) cells and promotes the survival and division of naive T cells or memory T cells. Furthermore, IL-7 enhances immune response in human by promoting the secretion of IL-2 and Interferon-y. The receptor of IL-7 is heterodimeric and consists of the IL-7Rα (CD127) and the common γ chain (CD132). The γ chain is expressed on all hematopoietic cell types whereas IL-7Rα is mainly expressed by lymphocytes that include B and T lymphoid precursors, naïve T cells and memory T cell. A low expression of IL-7Rα is observed on regulatory T cells compared to effector/naive T cells that express a higher level. Thereby, CD127 is used as surface marker to discriminate these 2 populations. IL-7Rα is also expressed on Innate lymphoid cells as NK and gut-associated lymphoid tissue (GALT)-derived T cells. IL-7Rα (CD127) chain is shared with TSLP (Tumor stromal lymphopoietin) and CD132 (y chain) is shared with IL-2, IL-4, IL-9, IL-15 and interleukin-21. Two main signaling pathways are induced through CD127/CD132: (1) Janus kinase/STAT pathway (i.e. Jak-Stat-3 and 5) and (2) the phosphatidyl-inositol-3kinase pathway (i.e. Pl3K-Akt). IL-7 administration is well tolerated in patient and leads to CD8 and CD4 cell expansion and a relative decrease of CD4+ T regulatory cells. Recombinant naked IL-7 or IL-7 fused to N terminal domain of the Fc of antibodies have been tested in clinic, with the rationale to increase IL-7 half-life via fusion of the Fc domain and enhance long lasting efficiency of the treatment.

Recombinant IL-7 cytokine has a poor pharmacokinetic profile limiting its use in clinic. After injection, recombinant IL-7 is rapidly distributed and eliminated leading to a poor half-life of IL-7 in human (ranging from 6.8 to 9.5 hours) (Sportes et al., Clin Cancer Res. 2010 Jan 15;16(2):727-35) or in mice (2.5 hours) (Hyo Jung Nam et al., Eur. J. Immunol. 2010. 40:351-358). A fusion of IgG Fc domain to IL-7 extends its half-life since the IgG can bind neonatal Fc receptor (FcRn) and engage transcytosis and endosomal recycling of the molecule. A prolonged circulating half-life is observed for the IL-7 Fc fusion molecule (t½=13 h) that remains at detectable levels (200 pg/mL) up to 8 days after administration in mice (Hyo Jung Nam et al., Eur. J. Immunol. 2010. 40:351-358). Although the half-life is increased for IL-7 cytokine fused to a Fc domain, the molecule required frequent in vivo injections to have a biological effect. In the context of immunocytokine molecules, the cytokine is fused to an antibody (e.g. targeting cancer antigen, immune checkpoint blockade, costimulatory molecule...) to preferentially concentrate the cytokine to the targeted antigen-expressing cells. However, the affinity of IL-7 cytokine for its CD127/CD132 receptor (nanomolar to picomolar range) may be higher than the affinity of the antibody for its target. Hence, the cytokine will drive the pharmacokinetics of the product leading to a fast depletion of the available drug in vivo due to the target-mediated drug disposition (TMDD) mechanism. This rapid elimination has been described for immunocytokine like IL-2 or IL-15 showing that pharmacokinetic properties of the fusion protein may directly impact on drug performance (List et Neri Clin Pharmacol. 2013; 5(Suppl 1): 29-45).

Then, it remains therefore a significant need in the art for new and improved IL-7 variant that allows to improve the distribution and reduce elimination of IL-7 products, particularly of bifunctional molecule comprising IL-7. The inventors have made a significant step forward with the invention disclosed herein.

### SUMMARY OF THE INVENTION

The inventors provide IL-7 mutations and optimized Fc backbones in order to improve the distribution and elimination of a bifunctional molecule for an enhanced biological effect in vivo. The inventors observed that IL-7 mutations; particularly in combination with the IgG isotype and linker length, allows a better distribution of the bifunctional molecule and a longer half-life in vivo.

The bifunctional molecules provided herein particularly demonstrates a good pharmacokinetics and pharmacodynamics in vivo, particularly in comparison with bifunctional molecule comprising an IL-7 wild type. In addition, advantageous and unexpected properties have been associated to these new molecules as detailed in the introduction of the detailed description and in the examples.

In a first aspect, the invention relates to a bifunctional molecule comprising an interleukin 7 (IL-7) variant conjugated to a binding moiety, wherein:
the binding moiety binds to a target specifically expressed on immune cells surface,
the IL-7 variant presents at least 75% identity with a wild type human IL-7 (wth-IL-7) comprising or consisting of the amino acid sequence set forth in SEQ ID NO: 1, wherein the variant comprises at least one amino acid mutation which i) reduces affinity of the IL-7 variant for IL-7 receptor (IL-7R) in comparison to the affinity of wth-IL-7 for IL-7R, and ii) improves pharmacokinetics of the bifunctional molecule comprising the IL-7 variant in comparison with a bifunctional molecule comprising wth-IL-7.

In particular, the at least one mutation is an amino acid substitution or a group of amino acid substitutions selected from the group consisting of (i) C2S-C141S and C47S-C92S, C2S-C141S and C34S-C129S, or C47S-C92S and C34S-C129S, (ii) W142H, W142F or W142Y, (iii) D74E, D74Q or D74N, iv) Q11E, Y12F, M17L, Q22E and/or K81R; or any combination thereof (i.e., the amino acid numbering being as shown in SEQ ID NO: 1).

In particular, the invention concerns bifunctional molecule comprising an interleukin 7 (IL-7) variant conjugated to a binding moiety, wherein:
the binding moiety binds to a target specifically expressed on immune cells surface,
the IL-7 variant presents at least 75% identity with a wild type human IL-7 (wth-IL-7) comprising or consisting of the amino acid sequence set forth in SEQ ID NO: 1, wherein the variant comprises at least one amino acid mutation selected from the group consisting of (i) W142H, W142F or W142Y, (ii) C2S-C141S and C47S-C92S, C2S-C141S and C34S-C129S, or C47S-C92S and C34S-C129S, (iii) D74E, D74Q or D74N, iv) Q11E, Y12F, M17L, Q22E and/or K81R; or any combination thereof, the amino acid numbering being as shown in SEQ ID NO: 1, which i) reduces affinity of the IL-7 variant for IL-7 receptor (IL-7R) in comparison to the affinity of wth-IL-7 for IL-7R, and ii) improves pharmacokinetics of the bifunctional molecule comprising the IL-7 variant in comparison with a bifunctional molecule comprising wth-IL-7.

In one aspect, the IL-7 variant comprises a group of amino acid substitutions selected from the group consisting of C2S-C141S and C47S-C92S, C2S-C141S and C34S-C129S, and C47S-C92S and C34S-C129S (i.e., the amino acid numbering being as shown in SEQ ID NO: 1).

In another aspect, the IL-7 variant comprises an amino acid substitution selected from the group consisting of W142H, W142F and W142Y (i.e., the amino acid numbering being as shown in SEQ ID NO: 1).

In another aspect, the IL-7 variant comprises in the amino acid substitution selected from the group consisting of D74E, D74Q and D74N (i.e., the amino acid numbering being as shown in SEQ ID NO: 1).

Particularly, the IL-7 variant comprises or consists of the amino acid sequence set forth in SEQ ID NO: 2-15.

In one aspect, the binding moiety comprises a heavy chain constant domain, preferably a Fc domain, of a human IgG1, optionally with a substitution or a combination of substitutions selected from the group consisting of T250Q/M428L; M252Y/S254T/T256E + H433K/N434F; E233P/L234V/L235A/G236A + A327G/A330S/P331S; E333A; S239D/A330L/l332E; P257I/Q311; K326W/E333S; S239D/I332E/G236A; N297A; L234A/L235A; N297A + M252Y/S254T/T256E; K322A and K444A, preferably selected from the group consisting of N297A optionally in combination with M252Y/S254T/T256E, and L234A/L235A.

Particularly, the binding moiety comprises a heavy chain constant domain, preferably a Fc domain, of a human IgG4, optionally with a substitution or a combination of substitutions selected from the group consisting of S228P; L234A/L235A, S228P + M252Y/S254T/T256E and K444A.

Preferably, the immune cell is a T cell, preferably an exhausted T cell.

In one aspect, the target is expressed by T cells and the binding moiety binds to a target selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8.

Preferably, the target is expressed by T exhausted cells and the binding moiety binds to a target preferably selected from the group consisting of PD-1, CTLA-4, BTLA, TIGIT, LAG3 and TIM3.

In one aspect, the binding moiety is an antibody or an antigen fragment thereof, and the N-terminal of the IL-7 variant is fused to the C-terminal of a heavy or light chain constant domain of the antibody or antibody fragment thereof, preferably to the C-terminal of the heavy chain constant domain, optionally via a peptide linker.

In another aspect, the IL-7 variant is fused to the binding moiety by a peptide linker selected from the group consisting of GGGGS (SEQ ID NO: 68), GGGGSGGGS (SEQ ID NO: 67), GGGGSGGGGS (SEQ ID NO: 69) and GGGGSGGGGSGGGGS (SEQ ID NO: 70), preferably is (GGGGS)₃ (SEQ ID NO: 70).

In one aspect, the molecule comprises a first monomer comprising an antigen-binding domain covalently linked via C-terminal end to N-terminal end of a first heterodimeric Fc chain optionally via a peptide linker, said first heterodimeric Fc chain being covalently linked by the C-terminal end to the N-terminal end of the IL-7 variant, optionally via a peptide linker, and a second monomer comprising a complementary second heterodimeric Fc chain devoid of antigen-binding domain. Preferably, in the second monomer, the complementary second heterodimeric Fc chain covalently linked to the IL-7 variant, optionally via a peptide linker, preferably covalently linked by C-terminal end to N-terminal of the IL-7 variant, optionally via a peptide linker.

In another aspect, the molecule comprises a first monomer comprising an antigen-binding domain covalently linked by C-terminal end to N-terminal end of a first heterodimeric Fc chain, optionally via a peptide linker, said first heterodimeric Fc chain being devoid of IL-7 variant, and a second monomer comprising a complementary second heterodimeric Fc chain devoid of antigen-binding domain, said second heterodimeric Fc chain being covalently to the IL-7 variant, optionally via a peptide linker, preferably linked by C-terminal end to N-terminal of the IL-7 variant, optionally via a peptide linker.

In another aspect, the molecule comprises a first monomer comprising an antigen-binding domain covalently linked via C-terminal end to N-terminal end of a first heterodimeric Fc chain optionally via a peptide linker, and a second monomer comprising an antigen-binding domain covalently linked via C-terminal end to N-terminal end of a complementary second heterodimeric Fc chain optionally via a peptide linker, wherein only one of heterodimeric Fc chains, preferably the first one, is covalently linked by the C-terminal end to the N-terminal end of the IL-7 variant.

Particularly, the antigen-binding domain of the bifunctional molecule is a Fab domain, a Fab′, a single-chain variable fragment (scFV) or a single domain antibody (sdAb).

Preferably, the antigen-binding domain comprises or consists essentially of: (i) a heavy chain comprising a CDR1 of SEQ ID NO: 51, a CDR2 of SEQ ID NO: 53 and a CDR3 of SEQ ID NO: 55,56, 57, 58, 59, 60, 61 or 62; and (ii) a light chain comprising a CDR1 of SEQ ID NO: 64 or SEQ ID NO: 65, a CDR2 of SEQ ID NO: 66 and a CDR3 of SEQ ID NO: 16.

Particularly, the antigen-binding domain comprises or consists essentially of:
(a) a heavy chain variable region (VH) comprising or consisting of an amino acid sequence of SEQ ID NO: 18, 19, 20, 21, 22, 23, 24 or 25;
(b) a light chain variable region (VL) comprising or consisting of an amino acid sequence of SEQ ID NO: 27 or SEQ ID NO: 28.

Preferably, the antigen-binding domain comprises or consists essentially of a heavy chain variable region (VH) of SEQ ID NO: 24 and a light chain variable region (VL) of SEQ ID NO: 28.

The invention also relates to an isolated nucleic acid sequence or a group of isolated nucleic acid molecules encoding the bifunctional molecule according to the invention.

The invention also concerns a host cell comprising the isolated nucleic acid according to the invention.

The invention also concerns a pharmaceutical composition comprising the bifunctional molecule, the nucleic acid or the host cell according to the invention, optionally with a pharmaceutically acceptable carrier.

The invention finally concerns the bifunctional molecule, the nucleic acid, the host cell or the pharmaceutical composition according to the invention, for use as a medicament, especially for use in the treatment of a cancer or an infectious disease.

### BRIEF DESCRIPTION OF THE DRAWINGS

FIG. 1: PD-1 binding ELISA assay. Human recombinant PD-1 (rPD1) protein was immobilized and antibodies were added at different concentrations. Revelation was performed with an anti-human Fc antibody coupled to peroxidase. Colorimetry was determined at 450 nm using TMB substrate. A. PD-1 binding of the bifunctional molecule comprising an anti-PD1 antibody and an IL-7 mutated on the amino acid D74, Q22, M17, Q11, K81. B. PD-1 binding of the bifunctional molecule comprising an IL-7 mutated on the amino acid W142 C. PD-1 binding of the bifunctional molecule mutated in the disulfide bonds of IL-7 (SS1, SS2 and SS3 mutant). All molecules tested in this figure were constructed with an lgG4m isotype and a GGGGSGGGGSGGGGS linker between the Fc and IL-7 domain.

FIG. 2: CD127 binding ELISA assay of IgG fused mutated IL-7. PD-1 recombinant protein was immobilized on the plate, then bifunctional anti-PD-1 IL-7 molecules were preincubated with CD127 recombinant protein (Histidine tagged, Sino ref 10975-H08H) and added to the well. Revelation was performed with a mixture of an anti-histidine antibody coupled to biotin + streptavidin coupled to Peroxidase. Colorimetry was determined at 450 nm using TMB substrate. A. CD127 binding of the bifunctional molecule comprising IL-7 mutated on the amino acid D74, Q22, M17, Q11, K81. B. CD127 binding of the bifunctional molecule comprising IL-7 mutated on the amino acid W142.

FIG. 3: IL-7R signaling pathway of the different bifunctional molecules as measured by STAT5 phosphorylation. Human PBMCs isolated from peripheral blood of healthy volunteers were incubated 15 minutes with bifunctional anti-PD-1 IL-7 molecules. Cells were then fixed, permeabilized and stained with an AF647 labeled anti-pSTAT5 (clone 47/Stat5(pY694)). Data were obtained by calculating MFI pSTAT5 in CD3 T cells. A. pSTAT5 activation of the anti-PD-1 IL-7 bifunctional molecule comprising an IL-7 mutated on the amino acid D74, Q22, M17, Q11, K81. B. pSTAT activation of the anti PD-1 IL-7 bifunctional molecule comprising an IL-7 mutated on the amino acid W142 C. pSTAT5 activation of the anti PD-1 IL-7 bifunctional molecule comprising an IL-7 mutated in the disulfide bonds of IL-7, SS2 (• black) and SS3 (▲) in comparison to anti PD-1 IL-7 WT (• grey). All molecules tested in this figure were constructed with an lgG4m isotype and a GGGGSGGGGSGGGGS linker between the Fc and IL-7 domain.

FIG. 4: Pharmacokinetics in mice of the anti PD-1 IL-7 bifunctional molecules Mice were intravenously injected with one dose with IgG fused IL-7 wild type or mutated IL-7. Concentration of the molecule in the sera was assessed by ELISA at multiple time points following injection. A. injection of IgG4-G4S3 IL7 WT (■ grey); IgG4-G4S3 IL7 D74E (•black) B. injection of IgG4-G4S3 IL7 WT (■ grey) or IgG4-G4S3 IL7 W142H (•black) C. injection of IgG4-G4S3 IL7 WT (■ grey); IgG4-G4S3 IL7 SS2 (•) or IgG4-G4S3 IL7 SS3 (▲). D. Correlation between Area under the curve (AUC) calculated from PK vs ED50 pSTAT5 (nM) of each molecule. All molecules tested in this figure were constructed with an IgG4m isotype and a GGGGSGGGGSGGGGS linker between the Fc and IL-7 domain.

FIG. 5: The addition of a disulfide bond between anti PD-1 and IL-7 decreases pSTAT5 activation while it increases drug exposure in vivo. A. IL7R signaling as measured by pSTAT5 activation on human PBMCs after treatment with anti PD-1 IL-7 bifunctional molecule WT (grey ●) or anti PD-1 IL-7 bifunctional molecule with an additional disulfide bond (black ●) B. Pharmacokinetics in mice of the anti PD-1 IL-7 bifunctional molecule WT (grey ●) or anti PD-1 IL-7 bifunctional molecule with an additional disulfide bond (black ●) molecules. Mice were intravenously injected with one dose with ant PD-1 IL7 bifunctional molecules. Concentration of the molecule in the sera was assessed by ELISA at multiple time points following injection. All molecules tested in this figure were constructed with an lgG4m isotype and a GGGGSGGGGSGGGGS linker between the Fc and IL-7 domain.

FIG. 6: PD-1 binding ELISA assay. Human recombinant PD-1 (rPD1) protein was immobilized and antibodies were added at different concentrations. Revelation was performed with an anti-human Fc antibody coupled to peroxidase. Colorimetry was determined at 450 nm using TMB substrate. A. PD-1 binding of the anti PD-1 IL-7 WT bifunctional molecule with an lgG4m (● grey), anti PD-1 IL-7 WT bifunctional molecule with an IgG1m (▲black), the anti PD-1 IL-7 D74E bifunctional molecule with an IgG1m isotype (■) or anti PD-1 IL-7 W142H bifunctional molecule with an IgG1m (◊). B. in another experiment, PD-1 binding of the anti PD-1 IL-7 SS2 bifunctional molecule with an lgG4m isotype (■) or anti PD-1 IL-7 SS2 bifunctional molecule with an IgG1m (▲) were tested.

FIG. 7: CD127 binding ELISA assay of anti PD-1 IL-7 bifunctional molecule constructed with an IgG1N298A or IgG4 isotype. Recombinant protein targeted by the antibody backbone was immobilized, then antibodies fused to IL-7 were preincubated with CD127 recombinant protein (Histidine tagged, Sino ref 10975-H08H). Revelation was performed with a mixture of an anti-histidine antibody coupled to biotin and streptavidin coupled to Peroxidase. Colorimetry was determined at 450 nm using TMB substrate. A. CD127 binding of anti PD-1 IL-7 W142H bifunctional molecule with an lgG4m isotype (● grey), anti PD-1 IL-7 W142H bifunctional molecule with an IgG1m (▲ black), or the anti PD-1 IL-7 WT bifunctional molecule with an IgG1m isotype (● black). B. CD127 binding of the anti PD-1 IL-7 SS2 bifunctional molecule with an lgG4m isotype (● grey), anti PD-1 IL-7 SS2 bifunctional molecule with an IgG1m (▲ black) or the anti PD-1 IL-7 WT bifunctional molecule with an IgG1m (● black). C. CD127 binding of the anti PD-1 IL-7 SS3 bifunctional molecule with an lgG4m isotype (● grey), anti PD-1 IL-7 SS3 bifunctional molecule with an IgG1m (▲ black) or the anti PD-1 IL-7 WT bifunctional molecule IgG1m (● black) D. CD127 binding of the anti PD-1 W142H bifunctional molecule with an isotype IgG1m (● black) or an isotype IgG1m + YTE (● grey). The CD127 binding the anti PD-1 D74E bifunctional molecule with an isotype IgG1m (▲ black) or an isotype IgG1m + YTE (▲ grey) were also tested. All molecules tested in this figure were constructed with a GGGGSGGGGSGGGGS linker between the Fc and IL-7 domain.

FIG. 8: IL-7R signaling analysis of anti PD-1 IL-7 bifunctional molecule constructed with an IgG1N298A or IgG4 isotype. humans PBMCs or Jurkat PD1+ CD127+ cells were incubated 15 minutes with anti PD-1 IL7 bifunctional molecule. Cells were then fixed, permeabilized and stained with an AF647 labeled anti-pSTAT5 (clone 47/Stat5(pY694)). Data were obtained by calculating % of pSTAT5 in CD3 T cells. A. pSTAT5 signaling on human PBMCs after treatment of the bifunctional molecule anti PD-1 IL-7 having the mutation D74E with an lgG4m isotype (● grey) or an IgG1m isotype (▲ black) B. pSTAT5 signaling on human PBMCs after treatment of the anti PD-1 IL-7 SS2 with an lgG4m isotype (● grey) or anti PD-1 IL-7 SS2 with an IgG1m (▲ black) ) C. pSTAT5 signaling on human PBMCs after treatment of the anti PD-1 IL-7 SS3 with an lgG4m isotype (● grey) or an IgG1m (▲ black) D. (left panel) pSTAT5 signaling on Jurkat PD1+CD127+ cells after treatment of the anti PD-1 IL-7 WT or anti PD-1 IL7 SS2 constructed with an lgG4m (● grey) or IgG1m (▲ black) isotype. D. (right panel) pSTAT5 signaling after treatment of the anti PD-1 IL-7 SS2 with an lgG4m isotype (● grey) or anti PD-1 IL-7 SS2 with an IgG1m (▲ black).

FIG. 9: Anti PD-1 IL-7 mutated bifunctional molecule potentiates T cell activation in vitro. Promega PD-⅟PD-L1 bioassay: (1) Effector T cells (Jurkat stably expressing PD-1, NFAT-induced luciferase) and (2) activating target cells (CHO K1 cells stably expressing PDL1 and surface protein designed to activate cognate TCRs in an antigen-independent manner) were co-cultured. After adding BioGlo™ luciferin, luminescence is quantified and reflects T cell activation. Serial molar concentration of anti-PD1 antibody +/- recombinant IL-7 (rIL-7) or anti-PD1IL7 bifunctional molecules were tested. Each dot represents EC50 of one experiment A. NFAT activation of the anti PD-1 IL-7 WT bifunctional molecule with an IgG4m isotype (● grey) or anti PD-1 (▲) or anti PD-1 + rIL-7 (○) B. NFAT activation of anti PD-1 IL-7 D74E IgG4m (●), PD-1 IL-7 D74E IgG1m (▲ dotted line), and anti PD-1 alone (black ▲) C. NFAT activation of anti PD-1 IL-7 W142H bifunctional molecule with IgG4m (●), PD-1 IL-7 W142H bifunctional molecule with IgG1m (▲ dotted line), and anti PD-1 alone (black ▲) D. NFAT activation of anti PD-1 IL-7 SS2 bifunctional molecule with IgG4m (●), and anti PD-1 alone (black ▲).

FIG. 10: Pharmacokinetics of anti PD-1 IL-7 bifunctional molecules constructed with an IgG1m or IgG4m isotype. Mice were intravenously injected with one dose with IgG fused to IL-7 wild type or to mutated IL-7. Concentration of the drug in the sera was assessed by ELISA at multiple time point following injection. A. Pharmacokinetics of the anti PD-1 IL-7 WT bifunctional molecule with lgG4m (● grey plain line), the anti PD-1 IL-7 WT bifunctional molecule with IgG1m (● grey dashed line), the anti PD-1 IL-7 D74E bifunctional molecule with IgG1m (▲ black dashed line), the anti PD-1 IL-7 W142H bifunctional molecule with lgG4m (○ black plain line), the anti PD-1 IL-7 W142H bifunctional molecule with IgG1m (● dashed black plain line), the anti PD-1 IL-7 SS3 with IgG4 (■ plain line), and the anti PD-1 IL-7 SS3 with IgG1m (■ dashed line). B. Pharmacokinetics of anti PD-1 IL-7 D74E, D74Q, W142H, D74E+W142H mutant bifunctional molecules with an IgG1m.

FIG. 11: Pharmacokinetics of anti PD-1 IL-7 bifunctional molecule constructed with an IgG1 N298A+K444A isotype. Mice were intravenously injected with one dose anti PD-1 IL7 D74E bifunctional molecule with an isotype IgG1N298A (■) or an isotype IgG1m+ K444A mutation isotype (●). Concentration of the antibody was assessed by ELISA at multiple time point following injection.

FIG. 12: Length of the linker does not significantly impact pharmacokinetics but decreases the stimulation of IL-7R signaling. A. Pharmacokinetics of anti PD-1 IL-7 WT bifunctional molecules constructed with different linkers (GGGGS), (GGGGS)2, (GGGGS)3). B. Pharmacokinetics of anti PD-1 IL-7 D74 bifunctional molecules constructed with different linkers (GGGGS), (GGGGS)2, (GGGGS)3). C. Pharmacokinetics of anti PD-1 IL-7 W142H bifunctional molecules constructed with different linkers ((GGGGS)2, (GGGGS)3). Mice were intravenously injected with one dose with IgG fused to IL-7 wild type or mutated IL-7. Concentration of the IgG fused to IL-7 was assessed by ELISA at multiple time points following injection. D. pSTAT5 signaling of the anti PD-1 IL-7 bifunctional molecules constructed without linker or with GGGGS, (GGGGS)2, (GGGGS)3 linkers.

FIG. 13: the anti PD-1 IL-7 mutant preferentially target PD-1+ CD127+ cells over PD-1-CD127+ cells

Jurkat cells expressing CD127+ or co-expressing CD127+ and PD-1+ were stained with 45 nM of anti PD-1 IL-7 bifunctional molecule and revealed with an anti IgG-PE (Biolegend, clone HP6017). Data represent ratio of the Median fluorescence on PD-1+CD127+ Jurkat cells over the Median fluorescence obtained on PD1- cells CD127+ Jurkat cells. In this assay, anti PD-1 IL-7 WT bifunctional molecule IgG1m, anti PD-1 IL-7 D74E bifunctional molecule IgG1m, anti PD-1 IL-7 W142H bifunctional molecule IgG1m, anti PD-1 IL-7 SS2 bifunctional molecule IgG4m, anti PD-1 IL-7 SS3 bifunctional molecule IgG1m were tested.

FIG. 14: The anti PD-1 IL-7 mutants preferentially target PD-1+ CD127+ cells over PD-1-CD127+ cells in a coculture assay. A. Expression was analyzed by flow cytometry of human CD127 and human PD-1 on the CHO cells transduced with CD127 only or with both CD127 and PD-1 receptors B. Binding of the anti PD-1 IL-7 mutants on CHO cells expressing CD127+ or co-expressing CD127+ and PD-1+ in a coculture assay. Cells were stained with a cell proliferation dye (CPDe450 or CPDe670), then co-cultivated at a ratio 1:1 prior incubation with different concentrations of anti PD-1 IL-7 bifunctional molecules. Revelation was performed with an anti IgG-PE (Biolegend, clone HP6017) and analyzed by flow cytometry. EC50 (nM) binding of each constructions on each cell type (CHO PD-1+ CD127+ (white histogram) and CHO PD-1-CD127+ (black histogram)) was calculated and reported. Histograms represent mean +/- SD of 3 independent experiments. In this assay, irrelevant mAb IL7 WT (isotype control) molecule IgG4m, anti PD-1 IL-7 W142H bifunctional molecule IgG1m, anti PD-1 IL-7 SS2 bifunctional molecule IgG4m, anti PD-1 IL-7 SS3 bifunctional molecule IgG1m were tested and comprise a GGGGSGGGGSGGGGS linker between the Fc and the IL-7 domain.

FIG. 15: The anti PD-1 IL-7 mutants preferentially activate pSTAT5 signaling into PD-1+ CD127+ cells over PD-1-CD127+ cells in a coculture assay. A. Expression analyzed by flow cytometry of human CD127, human PD-1 and human CD132 on U937 cells transduced with CD127 only or CD127 and PD-1 receptors B. pSTAT5 activity of the anti PD-1 IL-7 mutants in a coculture assay with U937 cells expressing CD127+ or co-expressing CD127+ and PD-1+. Cells were stained with a cell proliferation dye (CPDe450 or CPDe670) and co-cultivated at a ratio 1:1 prior incubation with different concentrations of anti PD-1 IL-7 bifunctional molecules (15 min 37° C.). Cells were then fixed, permeabilized and stained with an AF647 labeled anti-pSTAT5 (clone 47/Stat5(pY694). pSTAT5 activation EC50 (nM) was calculated for each construction and each cell type (CHO PD-1+ CD127+ (white histogram) and CHO PD-1-CD127+ (black histogram)). Histograms represent mean +/- SD of 4 independent experiments. In this assay, rIL-7 (recombinant human IL-7 cytokine), irrelevant mAb IL7 WT (isotype control) molecule IgG4m, anti PD-1 IL-7 D74E bifunctional molecule IgG1m, anti PD-1 IL-7 W142H bifunctional molecule IgG1m, anti PD-1 IL-7 SS2 bifunctional molecule IgG4m, anti PD-1 IL-7 SS3 bifunctional molecule IgG1m were tested and comprise a GGGGSGGGGSGGGGS linker between the Fc and IL-7 domains.

FIG. 16: The anti PD-1 IL-7 W142H mutant preferentially activates pSTAT5 signaling into PD-1+ CD127+ human T cells and synergistically increases proliferation of PD-1+CD127+ exhausted human T cells. Human PBMCs were stimulated on CD3/CD28 coating (3 µg/mL OK3 and CD28.2 antibody) to induce PD-1 expression, then pSTAT5 activity and proliferation were assessed with anti PD-1 IL-7 W142H bifunctional molecules IgG1m A. Left graph. Representative expression of human CD127, human PD-1 on activated human T cells (CD3+ population) analyzed by flow cytometry; A. right graph. human activated T cells were preincubated with isotype control or anti PD-1 competitive antibody (200 µg/mL) prior incubation with recombinant IL-7 or anti PD-1 IL-7 W142H mutant molecules. IL-7 R signaling pSTAT5 was quantified by flow cytometry after fixation and staining with AF647 labeled anti-pSTAT5 (clone 47/Stat5(pY694). pSTAT5 activation (EC50) was calculated in a condition with the isotype control and in a condition with the anti PD-1 competitive antibody. Data represent the fold-change difference between these 2 conditions; n= 5 different donors tested in independent experiments B. Proliferation of human exhausted PD-1+ T cells with an isotype control, an anti PD-1 + isotype IL7 W142H IgG1m or the anti PD-1 IL-7 W142H bifunctional molecule IgG1m (3 nM). Proliferation was measured on Day 5 following restimulation with αCD3/ PD-L1 recombinant protein coated plate. Proliferation was quantified by flow cytometry using a click-it EDU assay (geomean and % click-it EDU + cells); n= 4 independent T cell donors were tested in independent experiments. All constructions tested comprise a GGGGSGGGGSGGGGS linker between the Fc and IL-7 domains.

FIG. 17: Schematic representation of the different molecules used in Examples 8 and 9.

FIG. 18: Anti PD-1 IL7 W142H mutant demonstrates high binding efficiency to PD-1 and antagonizes PDL1 binding. A. PD-1 binding ELISA assay. Human recombinant PD-1 (rPD1) protein was immobilized, and antibodies were added at different concentrations. Revelation was performed with an anti-human Fc antibody coupled to peroxidase. Colorimetry was determined at 450 nm using TMB substrate. The anti PD-1 with 1 (anti PD-1*1 ▲ grey) or 2 anti PD-1 arms (anti PD-1*2 ◆) were tested as control. The bifunctional molecules comprising an IL7 variant (anti PD-1*2 IL7 W142H*2 ● black), (anti PD-1*2 IL7 W142H*1 ■ black), (anti PD-1*1 IL7 W142H*2 ● grey), (anti PD-1*1 IL7 W142H*1 ▼ grey) were also tested. B. Antagonistic capacity to block PD-1/PD-L1 measured by ELISA. PD-L1 was immobilized, and the complex antibodies + biotinylated recombinant human PD-1 was added. This\ complex was generated with a fixed concentration of PD1 (0.6 µg/mL) and different concentrations of anti-PD1*2 IL7 W142H*1 (■ plain line), anti-PD1*2 IL7 W142H*2(o dashed line), anti PD-1*1 (grey ▲ dashed grey line), anti-PD1*1 IL7 W142H*2 (grey ● plain grey line) or anti-PD1*1 IL7 W142H*1 (grey ▼ plain grey line). All constructions tested comprise a GGGGSGGGGSGGGGS linker between the Fc and IL-7 domain.

FIG. 19: Anti PD-1 IL7 molecules constructed with one or two valences of anti PD-1 and one IL-7 W142H cytokine activate pSTAT5 with high efficacy. A. PD-1/CD127 binding of anti PD-1 IL-7 W142H bifunctional molecules. PD-1 Recombinant protein was immobilized, then different concentrations of bifunctional molecules and a fixed quantity of CD127 recombinant protein (Histidine tagged, Sino ref 10975-H08H) were added. Revelation was performed with a mixture of an anti-histidine antibody coupled to biotin and streptavidin coupled to Peroxidase. Colorimetry was determined at 450 nm using TMB substrate. The anti-PD1*2 IL7 W142H1*1 (■) or anti-PD1*2 IL7 W142H*2 (● grey) were tested. B. pSTAT5 signaling assay with anti PD-1*2 backbone fused to IL-7 W142*1 cytokine. Human PBMCs isolated from peripheral blood of healthy volunteers were incubated 15 minutes with anti-PD1*2 IL7 WT*2 (▼) or anti-PD1*2 IL7W142H*1 (■ dashed line). Cells were then fixed, permeabilized and stained with an anti CD3-BV421 and an anti-pSTAT5 AF647 (clone 47/Stat5(pY694)). Data were obtained by calculating MFI %pSTAT5 + cells into CD3+ population. C. pSTAT5 signaling assay after treatment with anti PD-1*1 IL7 W142H*1 (●) anti PD-1*2 IL7WT*2 (■) or anti-PD1*2 IL7 W142H*1 (▲). All W142H constructions tested comprise an IgG1m and a GGGGSGGGGSGGGGS linker between the Fc and IL-7 domain.

FIG. 20: Anti PD-1lL7 molecules constructed with one two valences significantly promote T cell proliferation in vivo. Mice were intraperitoneally injected with one dose (34 nM/kg) of anti PD-1 IL-7 W142H molecules (anti PD-1*2 IL7 W142H*1, anti PD-1*1 IL7 W142H*1, anti PD-1*1 IL7 W142H*2), or an isotype control. On Day 4, blood was collected, and T cells were stained with an anti CD3, anti CD8, anti CD4 and ki67 proliferation marker. Kl67 percentage was quantified in the CD3 CD4+ and CD3 CD8+ populations. Statistical significance (*p<0,05) was calculated with one-way ANOVA test for multiple comparisons with control mice, n=2 to 8 mice per group of 2 independent experiments.

FIG. 21: Anti PD-1*2 IL7*1, Anti PD-1*1 IL7*1, Anti PD-1*1 IL7*2 synergistically activate TCR signaling. Promega PD-⅟PD-L1 bioassay : (1) Effector T cells (Jurkat stably expressing PD-1, NFAT-induced luciferase) and (2) activating target cells (CHO K1 cells stably expressing PDL1 and surface protein designed to activate cognate TCRs in an antigen-independent manner) were co-cultured. After adding BioGlo™ luciferin, luminescence is quantified and reflects T cell activation. A. anti-PD1*2 (● black), anti PD-1*2 IL7 W142H*1 (O white) were added at serial concentrations. Isotype antibody was used as negative control of activation (■) B. Combination of anti-PD1*1 + isotype IL7 W142H*2 control (○ white dashed line), anti PD-1*1 IL7 W142H *2(● grey), anti PD-1*1 IL7 W142H*1 (○ grey) were added at serial concentrations. All W142H constructions tested comprise an IgG1m and a GGGGSGGGGSGGGGS linker between the Fc and IL-7 domains.

FIG. 22: Anti PD-1*2 IL7*1, Anti PD-1*1 IL7*1, Anti PD-1*1 IL7*2 W142H mutants preferentially bind and activate pSTAT5 signaling into PD-1+ CD127+ cells over PD-1-CD127+ cells. U937 cells expressing CD127+ or co-expressing CD127+ and PD-1+ cells were stained with a cell proliferation dye (CPDe450 or CPDe670) and co-cultivated at ratio 1:1 prior incubation with different concentrations of anti PD-1 IL-7 bifunctional molecules. Staining with and anti-human IgG PE and pSTAT5 activation was quantified after incubation by flow cytometry. A. EC50 binding (nM) was calculated for each cell type and each construction. B. EC50 pSTAT5 (nM) was calculated for each cell type and each construction. After treatment with bifunctional molecules, cells were then fixed, permeabilized and stained with an AF647 labeled anti-pSTAT5 (clone 47/Stat5(pY694). pSTAT5 activation. EC50 (nM) was calculated for each construction and each cell type U937 PD-1+ CD127+ (white histogram) and U937 PD-1- CD127+ (black histogram). n=2 independent experiments. In this assay, anti PD-1*2 IL7 W142*1, anti PD-1*1 IL7 W142*1 and anti PD-1*1 IL7 W142*2 were tested and comprise an IgG1m isotype and a GGGGSGGGGSGGGGS linker between the Fc and IL-7 domains.

FIG. 23: Pharmacokinetics of the Anti PD-1*2 IL7*1, Anti PD-1*1 IL7*1, Anti PD-1*1 IL7*2 W142H mutant molecules following intraperitoneal injection. humanized PD1 mice were intraperitoneally injected with one dose (34 nM/kg) of the anti PD-1*2 IL7 IL7*2 lgG4m (Δ), anti PD-1*2 IL7 W142H*1 IgG1m (▼), anti PD-1*1 IL7 W142H*1 IgG1m (● grey), or anti PD-1*1 IL7 W142H*2 IgG1m (○ grey). Concentration of the drugs in the sera was assessed by ELISA following injection until 48h.

### DETAILED DESCRIPTION OF THE INVENTION

### Introduction

The molecule according to the invention are bifunctional since they combine the specific effect of human interleukin 7 variant associated to the targeting of specific target expressed on immune cells.

As known by the one skilled in the art, tumoral cells may not sufficiently be eliminated by T cells due to a phenomenon called T cells exhaustion, observed in many cancers. As described for instance by Jiang, Y., Li, Y. and Zhu, B (Cell Death Dis 6, e1792 (2015)), exhausted T cells in tumor microenvironment can lead to overexpression of inhibitory receptors, decrease of effector cytokine production and cytolytic activity, leading to the failure of cancer elimination and generally to cancer immune evasion. Restoring exhausted T cells is then a clinical strategy envisioned for cancer treatment.

Numerous exhaustion factors are known in the art such as programmed cell death protein 1 (PD1), cytotoxic T-lymphocyte-associated protein 4 (CTLA-4), T cell membrane protein-3 (TIM3), and lymphocyte activation gene 3 protein (LAG3), expressed on the surface of immune cells, in particular T cells. The immunosuppressive environment is particularly induced by the interaction of such molecule and their counterpart expressed on the surface of tumoral cells. More particularly, PD-1 is one of the major inhibitory receptors regulating T-cell exhaustion. Indeed, T cells with high PD-1 expression have a decreased ability to eliminate cancer cells. Anti-PD1 therapeutic compounds, especially anti-PD1 antibody, are used clinically in the treatment of cancer for blocking the inhibiting effect of PD1-PDL1 interaction (PD1 on T cells and PDL1 on tumoral cells) and T cells exhaustion. However, anti-PD1 antibodies are not always sufficiently efficient to allow the « re »activation of exhausted T cells.

The inventors demonstrated that a bifunctional molecule comprising an IL-7 variant according to the present invention and a binding moiety blocking an immunosuppressive interaction (checkpoint inhibitor) surprisingly activates synergistically a NFAT pathway, the main pathway required for T cell activation. Indeed, a synergistic activation of T cells through TCR signaling has been observed. More particularly, it has been shown that bifunctional IL-7 variant - anti-PD-1 molecules lead to a better activation of T cells, in particular of exhausted T cells, when compared to the anti-PD-1 alone.

The inventors have now newly shown that the interaction of the bifunctional molecules with i) an exhaustion factor expressed at the surface of T cells such as PD1, CTLA-4, BTLA, TIGIT, LAG3 and TIM3 (for the binding moiety) and ii) IL7 receptor (for the IL7-variant side) on a same T cell, leads to this unexpected activation of the NFAT pathway (TCR signaling) with the positive effect of activation of T cells, and in particular exhausted T cells that would otherwise not be capable of eliminating tumoral cells. This effect has never been disclosed before.

In addition, the use of IL-7 variants in the bifunctional molecules is important to increase the pharmacokinetics in vivo. Furthermore, by decreasing the affinity of IL-7 variant for its receptor, it increases the capacity of the bifunctional molecules to preferentially bind the targeted immune cells and to present a specific effect on these cells in comparison to others but also to take advantage of the synergistic effect associated to the action of the two parts of the bifunctional molecule on the same immune cells. More particularly, it is thought that the bifunctional molecules comprising an IL-7 variant and a binding moiety targeting an exhaustion factor will allow accumulation of IL-7 in T cells infiltrates and re-localization of IL-7 on T cells. This accumulation of IL-7 near these T cells is of particular interest in the context of exhausted T cells which require high dose of IL-7 for activating or re-activating these T cells.

Surprisingly, the inventors observed that the bifunctional molecules having an IgG1 heavy chain constant domain have an improved activity of IL-7 variants (pStat5 signal, synergistic effect and CD127 binding) compared to the same molecule with an IgG4 heavy chain constant domain. This improvement is specific of the IL-7 mutants and has not been observed with the wildtype IL-7. In addition, the use of a linker (GGGGS)₃ between the antibody and the IL-7 maximizes the activity of IL-7 variants (pStat5 signal and CD127 binding).

The bifunctional molecules of the invention have in particular one or several of the following advantages:
The bifunctional molecules allow a specific localization of IL-7 variant close to immune cells such as T cells or PD-1+ cells, in particular into the tumor, targeting cells that require higher concentration of IL-7.
The mutation in the IL-7 variants decreases the affinity of IL-7 variants to IL-7R without the complete or significant loss of its intrinsic biological activity, in comparison to an IL-7 wild type.
The mutation in the IL-7 variants improves pharmacokinetics and pharmacodynamics in vivo, particularly in comparison with bifunctional molecule comprising an IL-7 wild type. More particularly, improving pharmacokinetics and pharmacodynamics of the molecule allows the bifunctional molecule to reach the targeted cells, and to act on the target expressed at the surface of the immune cells.
The bifunctional molecules according to the invention show synergistic activity of the IL7 mutant (NFAT signaling).
Bifunctional molecules according to the invention have highest selective activity towards PD-1(+) cells than PD-1(-) cells compared to antibodies comprising the wild type IL7.
Bifunctional molecules comprising a mutated IL-7 W142H molecule selectively and synergistically cis-activate PD-1(+) CD127(+) exhausted T cells.
The IL-7 variants may be included in several structures of bifunctional molecules having one or two IL-7 molecules and one or two antigen binding fragments while keeping capacity to bind their target (e.g. PD-1) and to activate the IL7R pathway. In particular, bifunctional molecules having 1 or 2 IL7 W142H variant have a good pharmacokinetic profile in vivo.
The inventors surprisingly show the improved properties of a construction comprising a single IL-7 variant compared to constructions comprising two IL7 variants, both in terms of activity and pharmacokinetics.

### Definitions

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

As used herein, the terms “wild type interleukin-7”, “wt-IL-7” and “wt-IL7” refers to a mammalian endogenous secretory glycoprotein, particularly IL-7 polypeptides, derivatives and analogs thereof having substantial amino acid sequence identity to wild-type functional mammalian IL-7 and substantially equivalent biological activity, e.g., in standard bioassays or assays of IL-7 receptor binding affinity. For example, wt-IL-7 refers to an amino acid sequence of a recombinant or non-recombinant polypeptide having an amino acid sequence of: i) a native or naturally-occurring IL-7 polypeptide, ii) a biologically active fragment of an IL-7 polypeptide, iii) a biologically active polypeptide analog of an IL-7 polypeptide, or iv) a biologically active IL-7 polypeptide. The IL-7 can comprise its peptide signal or be devoid of it. Alternative designations for this molecule are “pre-B cell growth factor” and “lymphopoietin-1”. Preferably, the term “wt-IL-7” refers to human IL-7 (wth-IL7). For example, the human wt-IL-7 amino acid sequence is about 152 amino acids (in absence of signal peptide) and has a Genbank accession number of NP_000871.1, the gene being located on chromosome 8q12-13. Human IL-7 is for example described in UniProtKB - P13232.

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. Unless specifically noted otherwise, the term “antibody” includes intact immunoglobulins and “antibody fragment” or “antigen binding fragment” (such as Fab, Fab′, F(ab′)2, Fv), single chain (scFv), mutants thereof, molecules comprising an antibody portion, diabodies, linear antibodies, single chain antibodies, and any other modified configuration of the immunoglobulin molecule that comprises an antigen recognition site of the required specificity, including glycosylation variants of antibodies, amino acid sequence variants of antibodies. Preferably, the term antibody refers to a humanized antibody.

An “antibody heavy chain” as used herein, refers to the larger of the two types of polypeptide chains present in antibody conformations. The CDRs of the antibody heavy chain are typically referred to as “HCDR1”, “HCDR2” and “HCDR3”. The framework regions of the antibody heavy chain are typically referred to as “HFR1”, “HFR2”, “HFR3” and “HFR4”.

An “antibody light chain,” as used herein, refers to the smaller of the two types of polypeptide chains present in antibody conformations; κ and λ light chains refer to the two major antibody light chain isotypes. The CDRs of the antibody light chain are typically referred to as “LCDR1”, “LCDR2” and “LCDR3”. The framework regions of the antibody light chain are typically referred to as “LFR1”, “LFR2”, “LFR3” and “LFR4”.

As used herein, an “antigen-binding fragment” of an antibody means a part of an antibody, i.e. a molecule corresponding to a portion of the structure of the antibody of the invention, that exhibits antigen-binding capacity for a particular antigen, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof.

As used herein, the term “humanized antibody” is intended to refer to antibodies in which CDR sequences derived from the germline of another mammalian species, such as a mouse, have been grafted onto human framework sequences (e.g. chimeric antibodies that contain minimal sequence derived from a non-human antibody). A “humanized form” of an antibody, e.g., a non- human antibody, also refers to an antibody that has undergone humanization. A humanized antibody is generally a human immunoglobulin (recipient antibody) in which residues from one or more CDRs are replaced by residues from at least one CDR of a non-human antibody (donor antibody) while maintaining the desired specificity, affinity, and capacity of the original antibody. Additional framework region modifications may be made within the human framework sequences. Preferably humanized antibody has a T20 humanness score greater than 80%, 85% or 90%. “Humanness” of an antibody can for example be measured using the T20 score analyzer to quantify the humanness of the variable region of antibodies as described in Gao S H, Huang K, Tu H, Adler A S. BMC Biotechnology. 2013: 13:55 or via a web-based tool to calculate the T20 score of antibody sequences using the T20 Cutoff Human Databases: abAnalyzer.lakepharma.com.

By “chimeric antibody” is meant an antibody made by combining genetic material from a nonhuman source, preferably such as a mouse, with genetic material from a human being. Such antibody derives from both human and non-human antibodies linked by a chimeric region. Chimeric antibodies generally comprise constant domains from human and variable domains from another mammalian species, reducing the risk of a reaction to foreign antibodies from a non-human animal when they are used in therapeutic treatments.

As used herein, the terms “fragment crystallizable region” “Fc region” or “Fc domain” are interchangeable and refers to the tail region of an antibody that interacts with cell surface receptors called Fc receptors. The Fc region or domain is typically composed of two identical domains, derived from the second and third constant domains of the antibody’s two heavy chains (i.e. CH2 and CH3 domains). Portion of the Fc domain refers to the CH2 or the CH3 domain. Optionally, the Fc region or domain may optionally comprise all or a portion of the hinge region between CH1 and CH2. Optionally, the Fc domain is that from IgG1, lgG2, lgG3 or lgG4, optionally with IgG1 hinge-CH2-CH3 and IgG4 hinge-CH2-CH3.

In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat.

By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e. starting from the N terminus) should be considered as having position 1.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A

Amino Acid Residue

| | |
|---|---|
| Amino Acid groups | Amino Acid Residues |
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B

Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C

Further Alternative Physical and Functional Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved in turn formation | A, C, D, E, G, H, K, N, Q, R, S, P, and T |
| Flexible residues | E, Q, T, K, S, G, P, D, E, and R |

As used herein, the “sequence identity” between two sequences is described by the parameter “sequence identity”, “sequence similarity” or “sequence homology”. For purposes of the present invention, the “percentage identity” between two sequences (A) and (B) is determined by comparing the two sequences aligned in an optimal manner, through a window of comparison. Said alignment of sequences can be carried out by well-known methods in the art, for example, using the algorithm for global alignment of Needleman-Wunsch. Protein analysis software matches similar sequences using measures of similarity assigned to various substitutions, deletions and other modifications, including conservative amino acid substitutions. Once the total alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two amino acid sequences, one can use, for example, the tool “Emboss needle” for pairwise sequence alignment of proteins providing by EMBL-EBI and available on: see Worldwide Website: ebi.ac.uk/Tools/services/web/toolform.ebi?tool=emboss_needle& context=protein, for example using default settings: (l) Matrix : BLOSUM62, (ii) Gap open : 10, (iii) gap extend : 0.5, (iv) output format: pair, (v) end gap penalty : false, (vi) end gap open : 10, (vii) end gap extend : 0.5.

Alternatively, Sequence identity can also be typically determined using sequence analysis software Clustal Omega using the HHalign algorithm and its default settings as its core alignment engine. The algorithm is described in Söding, J. (2005) ‘Protein homology detection by HMM-HMM comparison’. Bioinformatics 21, 951-960, with the default settings.

The terms “derive from” and “derived from” as used herein refers to a compound having a structure derived from the structure of a parent compound or protein and whose structure is sufficiently similar to those disclosed herein and based upon that similarity, would be expected by one skilled in the art to exhibit the same or similar properties, activities and utilities as the claimed compounds.

As used herein, a “pharmaceutical composition” refers to a preparation of one or more of the active agents, such as comprising a bifunctional molecule according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one embodiment, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g. the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain embodiments, such a term refers to the improvement or eradication of a disease, a disorder, an infection or symptoms associated with it. In other embodiments, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject who has a disorder/disease.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease e.g. an illness that disrupts normal physical or mental functions. More preferably, the term disorder refers to immune and/or inflammatory diseases that affect animals and/or humans, such as cancer.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT) and myeloid-derived cells (neutrophil, eosinophil, basophil, monocyte, macrophage, dendritic cells). In particular, the immune cell can be selected in the non-exhaustive list comprising B cells, T cells, in particular CD4+ T cells and CD8+ T cells, NK cells, NKT cells, APC cells, dendritic cells and monocytes. “T cell” as used herein includes for example CD4 + T cells, CD8 + T cells, T helper 1 type T cells, T helper 2 type T cells, T helper 17 type T cells and inhibitory T cells.

As used herein, the term “T effector cell”, “T eff” or “effector cell” describes a group of immune cells that includes several T cells types that actively respond to a stimulus, such as co-stimulation. It particularly includes T cells which function to eliminate antigen (e.g., by producing cytokines which modulate the activation of other cells or by cytotoxic activity). It notably includes CD4+, CD8+, cytotoxic T cells and helper T cells (Th1 and Th2).

As used herein, the term “regulatory T cell”, Treg cells” or “T reg” refers to a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25 and are thought to be derived from the same lineage as naïve CD4 cells.

The term “exhausted T cell” refers to a population of T cell in a state of dysfunction (i.e. “exhaustion”). T cell exhaustion is characterized by progressive loss of function, changes in transcriptional profiles and sustained expression of inhibitory receptors. Exhausted T cells lose their cytokines production capacity, their high proliferative capacity and their cytotoxic potential, which eventually leads to their deletion. Exhausted T cells typically indicate higher levels of CD43, CD69 and inhibitory receptors combined with lower expression of CD62L and CD127.

The term “immune response” refers to the action of, for example, lymphocytes, antigen presenting cells, phagocytic cells, granulocytes, and soluble macromolecules produced by the above cells or the liver (including antibodies, cytokines, and complements) that results in selective damage to, destruction of, or elimination from the human body of invading pathogens, cells or tissues infected with pathogens, cancerous cells, or, in cases of autoimmunity or pathological inflammation, normal human cells or tissues.

The term “antagonist” as used herein, refers to a substance that blocks or reduces the activity or functionality of another substance. Particularly, this term refers to an antibody that binds to a cellular receptor (e.g. PD-1) as a reference substance (e.g. PD-L1 and/or PD-L2), preventing it from producing all or part of its usual biological effects (e.g. the creation of an immune suppressive microenvironment). The antagonist activity of a humanized antibody according to the invention may be assessed by competitive ELISA.

The term “agonist” as used herein, refers to a substance that activates the functionality of an activating receptor. Particularly, this term refers to an antibody that binds to a cellular activating receptor as a reference substance, and have at least partially the same effect of the biologically natural ligand (e.g. inducing the activatory effect of the receptor).

Pharmacokinetics (PK) refers to the movement of drugs through the body, whereas pharmacodynamics (PD) refers to the body’s biological response to drugs. PK describes a drug’s exposure by characterizing absorption, distribution, bioavailability, metabolism, and excretion as a function of time. PD describes drug response in terms of biochemical or molecular interactions. PK and PD Analyses are used to characterize drug exposure, predict and assess changes in dosage, estimate rate of elimination and rate of absorption, assess relative bioavailability / bioequivalence of a formulation, characterize intra- and inter-subject variability, understand concentration-effect relationships, and establish safety margins and efficacy characteristics. By “improving PK” it is meant that one of the above characteristics is improved, for example, such as an increased half-life of the molecule, in particular a longer serum half-life of the molecule when injected to a subject.

As used herein, the term “isolated” indicates that the recited material (e.g., antibody, polypeptide, nucleic acid, etc.) is substantially separated from, or enriched relative to, other materials with which it occurs in nature. Particularly, an “isolated” antibody is one which has been identified and separated and/or recovered from a component of its natural environment.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/-10% (e.g., +/- 0.5%, +/-1 %, +/-1.5%, +/- 2%, +/- 2.5%, +/- 3%, +/- 3.5%, +/- 4%, +/- 4.5%, +/- 5%, +/-5.5%, +/- 6%, +/- 6.5%, +/- 7%, +/- 7.5%, +/- 8%, +/- 8.5%, +/- 9%, +/-9.5%). The use of the term “about” at the beginning of a string of values modifies each of the values (i.e. “about 1, 2 and 3” refers to about 1, about 2 and about 3). Further, when a listing of values is described herein (e.g. about 50%, 60%, 70%, 80%, 85% or 86%) the listing includes all intermediate and fractional values thereof (e.g., 54%, 85.4%).

### IL-7 Mutants

The present disclosure provides interleukin 7 mutants (IL-7m) and bifunctional molecules comprising a first entity that comprises an interleukin 7 mutant (IL-7m) and a second entity comprising a binding moiety.

The terms “interleukin-7 mutant”, “mutated IL-7”, “IL-7 mutant”, “IL-7 variant”, “IL-7m” or IL-7v” are used interchangeably herein. A “variant” or “mutant” of an IL-7 protein is defined as an amino acid sequence that is altered by one or more amino acids. The variant can have “conservative” modifications or “non-conservative” modifications. Such modifications can include amino acid substitution, deletions and/or insertions. Preferably, the modifications are substitutions, in particular conservative substitutions. The variant IL-7 proteins included within the invention specifically concern IL-7 proteins that do not retain substantially equivalent biological property (e.g. activity, binding capacity and/or structure) in comparison to a wild-type IL-7. The IL-7 mutant or variant comprises at least one mutation. Particularly, the at least one mutation decreases the affinity of IL-7m to IL-7R but do not lead to the loss of the recognition of IL-7R. Accordingly, the IL-7 mutant or variant retains a capacity to activate IL-7R, for instance as measured by the pStat5 signal, for example such as disclosed in Bitar et al., Front. Immunol., 2019, volume 10). The biological activity of IL-7 protein can be measured using in vitro cellular proliferation assays or by measuring the P-Stat5 into the T cells by ELISA or FACS. Preferably, the IL-7 variants according to the invention has reduced biological properties (e.g. activity, binding capacity and/or structure) by at least a factor 2, 5, 10, 20, 30, 40, 50, 100, 250, 500, 750,1000, 2500, 5000, or 8000 in comparison with the wild type IL-7, preferably the wth-IL7. More preferably, the IL-7 variants have a reduced binding to the IL-7 receptor but retains a capacity to activate IL-7R. For instance, the binding to the IL-7 receptor can be reduced by at least 10 %, 20%, 30%, 40%, 50%, 60% in comparison with the wild type IL-7, and retains a capacity to activate IL-7R by at least 90%, 80%, 70%, 60%, 50%, 40%, 30% or 20% in comparison with the wild type IL-7. Preferably, the IL-7m is a variant of the human wild type IL-7, for example such as described in SEQ ID NO: 1.

In one embodiment, the IL-7 variants according to the invention maintain biological activity by at least 1%, 5%, 10 %, 20%, 30%, 40%, 50%, 60% in comparison with the wild type human IL-7, preferably at least 80%, 90%, 95% and even more preferably 99% in comparison with the wild type IL-7.

In one aspect, the IL-7 variant or mutant differs from wt-IL-7 by at least one amino acid mutation which i) reduces affinity of the IL-7 variant for IL-7 receptor (IL-7R) in comparison to the affinity of wt-IL-7 for IL-7R, and ii) improves pharmacokinetics of the IL7 variant in comparison to the wt-IL7. More particularly, the IL-7 variant or mutant further retains the capacity to activate IL-7R, in particular through the pStat5 signaling.

In another aspect, the bifunctional molecule comprising an IL-7 variant or mutant differs from a wt-IL-7 by at least one amino acid mutation which i) reduces affinity of the bifunctional molecule for IL-7 receptor (IL-7R) in comparison to the affinity for IL-7R of a bifunctional molecule comprising wt-IL-7, and ii) improves pharmacokinetics of the bifunctional molecule comprising an IL-7 variant or mutant in comparison to the bifunctional molecule comprising wt-IL-7. More particularly, the bifunctional molecule comprising an IL-7 variant or mutant further retains the capacity to activate IL-7R, in particular through the pStat5 signaling. For instance, the binding bifunctional molecule comprising an IL-7 variant or mutant to the IL-7 receptor can be reduced by at least 10 %, 20%, 30%, 40%, 50%, 60% in comparison with the bifunctional molecule comprising a wild type IL-7, and retains a capacity to activate IL-7R by at least 90%, 80%, 70%, 60%, 50%, 40%, 30% or 20% in comparison with the bifunctional molecule comprising a wild type IL-7.

According to the invention, the IL-7m presents a reduced affinity for IL-7 receptor (IL-7R) in comparison to the affinity of wth-IL-7 for IL-7R. In particular, the IL-7m present a reduced affinity for CD127 and/or CD132 in comparison to the affinity of wth-IL-7 for CD127 and/or CD132, respectively. Preferably, the IL-7m presents a reduced affinity for CD127 in comparison to the affinity of wth-IL-7 for CD127.

Preferably, the at least one amino acid mutation decreases the affinity of IL-7m for IL-7R, in particular CD132 or CD127, by at least a factor 10, 100, 1000, 10000, or 100 000, in comparison to the affinity of wt-IL-7 for IL-7R. Such affinity comparison may be performed by any methods known by the skilled of the art, such as ELISA or Biacore.

Preferably, the at least one amino acid mutation decreases affinity of IL-7m for IL-7R but do not decrease the biological activity of IL-7m in comparison to IL-7 wt, in particular as measured by pStat5 signal.

Alternatively, the at least one amino acid mutation decreases affinity of IL-7m for IL-7R but do not decrease significatively the biological activity of IL-7m in comparison to IL-7 wt, in particular as measured by pStat5 signal.

Additionally or alternatively, the IL-7m improves pharmacokinetics of IL-7 variant or mutant or of the bifunctional molecule comprising the IL-7 variant in comparison with a wild-type IL-7 or a bifunctional molecule comprising a wild type IL-7, respectively. Particularly, the IL-7m according to the invention improves pharmacokinetics of the IL-7 variant by at least a factor 10, 100 or 1000 in comparison with a wth-IL-7. Particularly, the IL-7m according to the invention improves pharmacokinetics of the bifunctional molecule comprising IL-7 variant or mutant by at least a factor 10, 100 or 1000 in comparison with a bifunctional molecule comprising wth-IL-7. Pharmacokinetics profile comparison may be performed by any methods known by the skilled of the art, such as in vivo injection of the drug and dosage ELISA of the drug in the sera at multiple time point for example as shown in example 2.

As used herein, the terms “pharmacokinetics” and “PK” are used interchangeably and refer to the fate of compounds, substances or drugs administered to a living organism. Pharmacokinetics particularly comprise the ADME or LADME scheme, which stands for Liberation (i.e. the release of a substance from a composition), Absorption (i.e. the entrance of the substance in blood circulation), Distribution (i.e. dispersion or dissemination of the substance trough the body) Metabolism (i.e. transformation or degradation of the substance) and Excretion (i.e. the removal or clearance of the substance from the organism). The two phases of metabolism and excretion can also be grouped together under the title elimination. Different pharmacokinetics parameters can be monitored by the man skilled in the art, such as elimination half-life, elimination constant rate, clearance (i.e. the volume of plasma cleared of the drug per unit time), Cmax (Maximum serum concentration), and Drug exposure (determined by Area under the curve, see Scheff et al, Pharm Res. 2011 May;28(5):1081-9) among others.

Then, the improvement of the pharmacokinetics by the use of IL-7m, in particular in a bifunctional molecule, refers to the improvement of at least one of the above-mentioned parameters. Preferably, it refers to the improvement of the elimination half-life of the bifunctional molecule, i.e. the increase of half-life duration, or of Cmax.

In a particular embodiment, the at least one mutation of IL-7m improves the elimination half-life of a bifunctional molecule comprising IL-7m in comparison to a bifunctional molecule comprising IL-7 wt.

In one embodiment, the IL-7m presents at least 75%, at least 80%, at least 85%, at least 90%, at least 95%, at least 97%, at least 98% or at least 99% of identity with the wild-type human IL-7 (wth-IL-7) protein of 152 amino acids, such as disclosed in SEQ ID NO: 1. Preferably, the IL-7m presents at least 75%, at least 80%, at least 85%, at least 90%, at least 95%, at least 97%, at least 98% or at least 99% of identity with SEQ ID NO: 1.

Particularly, the at least one mutation occurs at amino acid position 74 and/or 142 of IL-7. Additionally or alternatively, the least one mutation occurs at amino acid positions 2 and 141, 34 and 129, and/or 47 and 92. These positions refer to the position of amino acids set forth in SEQ ID NO: 1.

Particularly, the at least one mutation is an amino acid substitution or a group of amino acid substitutions is selected from the group consisting of C2S-C141S and C47S-C92S, C2S-C141S and C34S-C129S, C47S-C92S and C34S-C129S, W142H, W142F, W142Y, Q11E, Y12F, M17L, Q22E, K81R, D74E, D74Q and D74N or any combination thereof. These mutations refer to the position of amino acid set forth in SEQ ID NO: 1. Then, for example, the mutation W142H stands for the substitution of tryptophan of the wth-IL7 into a histidine, to obtain an IL-7m having a histidine in amino acid position 142. Such mutant is for example described under SEQ ID NO: 5.

In one embodiment, the IL-7m comprises sets of substitutions in order to disrupt disulfide bonds between C2 and C141, C47 and C92, and C34-C129. In particular, the IL-7m comprises two sets of substitutions in order to disrupt disulfide bonds between C2 and C141, and C47 and C92; C2 and C141, and C34-C129; or C47 and C92, and C34-C129. For instance, the cysteine residues can be substituted by serine in order to prevent disulfide bonds formation. Accordingly, the amino acid substitutions can be selected from the group consisting of C2S-C141S and C47S-C92S (referred as “SS2”), C2S-C141S and C34S-C129S (referred as “SS1”), and C47S-C92S and C34S-C129S (referred as “SS3”). These mutations refer to the position of amino acids set forth in SEQ ID NO: 1. Such IL-7m are particularly described under the sequence set forth in SEQ ID NOs: 2 to 4 (SS1, SS2 and SS3, respectively). Preferably, the IL-7m comprises the amino acids substitutions C2S-C141S and C47S-C92S. Even more preferably, the IL-7m presents the sequence set forth in SEQ ID NO: 3.

In another embodiment, the IL-7m comprises at least one mutation selected from the group consisting of W142H, W142F, and W142Y. Such IL-7m are particularly described in under the sequence set forth in SEQ ID NOs: 5 to 7, respectively. Preferably, the IL-7m comprises the mutation W142H. Even more preferably, the IL-7m presents the sequence set forth in SEQ ID NO: 5.

In another embodiment, the IL-7m comprises at least one mutation selected from the group consisting of D74E, D74Q and D74N, preferably D74E and D74Q. Such IL-7m are particularly described in under the sequence set forth in SEQ ID NOs: 12 to 14, respectively. Preferably, the IL-7m comprises the mutation D74E. Even more preferably, the IL-7m presents the sequence set forth in SEQ ID NO: 12.

In another embodiment, the IL-7m comprises at least one mutation selected from the group consisting of Q11E, Y12F, M17L, Q22E and/or K81R. These mutations refer to the position of amino acids set forth in SEQ ID NO: 1. Such IL-7m are particularly described in under the sequence set forth in SEQ ID NOs: 8, 9, 10, 11, and 15, respectively.

In one embodiment, the IL-7m comprises at least one mutation that consists in i) W142H, W142F or W142Y and/or ii) D74E, D74Q or D74N, preferably D74E or D74Q and/or iii) C2S-C141S and C47S-C92S, C2S-C141S and C34S-C129S, or C47S-C92S and C34S-C129S.

In one embodiment, the IL-7m comprises the W142H substitution and at least one mutation consisting of i) D74E, D74Q or D74N, preferably D74E or D74Q and/or ii) C2S-C141S and C47S-C92S, C2S-C141S and C34S-C129S, or C47S-C92S and C34S-C129S.

In one embodiment, the IL-7m comprises the D74E substitution and at least one mutation consisting of i) W142H, W142F or W142Y and/or ii) C2S-C141S and C47S-C92S, C2S-C141S and C34S-C129S, or C47S-C92S and C34S-C129S.

In one embodiment, the IL-7m comprises the mutations C2S-C141S and C47S-C92S and at least one substitution consisting of i) W142H, W142F or W142Y and/or ii) D74E, D74Q or D74N, preferably D74E or D74Q.

In one embodiment, the IL-7m comprises i) D74E and W142H substitutions and ii) the mutations C2S-C141S and C47S-C92S, C2S-C141S and C34S-C129S, or C47S-C92S and C34S-C129S.

The IL-7m proteins can comprise its peptide signal or be devoid of it. A variant of IL-7 may also include altered polypeptides sequence of IL-7 (e.g. oxidized, reduced, deaminated or truncated forms).

In one aspect, the IL-7 variant or mutant used in the present invention is a recombinant IL-7. The term “recombinant”, as used herein, means that the polypeptide is obtained or derived from a recombinant expression system, i.e., from a culture of host cells (e.g., microbial or insect or plant or mammalian) or from transgenic plants or animals engineered to contain a nucleic acid molecule encoding an IL-7m polypeptide. Preferably, the recombinant IL-7 is a human recombinant IL-7m, (e.g. a human IL-7m produced in recombinant expression system).

In one embodiment, the IL-7m present the sequence set forth in SEQ ID NO: 2, 3, 4, 5, 6, 7, 8, 9, 10, 11, 12, 13, 14 or 15. Preferably, the bifunctional molecule according to the invention comprises an IL-7 variant that comprises or consists of the amino acid sequence set forth in SEQ ID NO: 2-15. Even more preferably, the bifunctional molecule according to the invention comprises an IL-7 variant that comprises or consists of the amino acid sequence set forth in SEQ ID NO: 3, 5 or 12.

In one embodiment, the invention provides IL-7 variants and bifunctional molecules comprising IL-7 variants, that have a reduced immunogenicity compared to wild-type IL-7 proteins, particularly by the removing T-cell epitopes within IL-7 that may stimulate to an immune response. Examples of such IL-7 are described in WO 2006061219.

The present invention also relates to any fusion protein comprising the IL-7 variants or mutants as disclosed herein and to any conjugate comprising the IL-7 variants or mutants as disclosed herein. The IL-7 variants or mutants can be fused by their N-terminal end or their C-terminal end. The IL-7 variants or mutants can be fused or conjugated to a peptide, a protein (e.g., antibody, fragment and derivative thereof, antibody mimics, cytokine or cytokine receptor, tumor or viral antigens, albumin or albumin binding protein), a polymer (e.g. PEG), a chemical compound such as a drug (e.g., anticancer or antiviral agent), a carbohydrate and a nucleic acid molecule (e.g., siRNA, shRNA, antisense, Gapmer).

A non-exhaustive list of molecules that can be conjugated or fused to IL-7 variants or mutants include an antibody such as an anti-CD19, an anti-calreticulin, an anti-tumor antigen; a cytokine or a cytokine receptor such as IL-15 or IL-15R; a domain which prolongs the half-life of IL-7 variant such as an Fc region of immunoglobulin or a part thereof, albumin, an albumin-binding polypeptide, Pro/Ala/Ser (PAS), a C-terminal peptide (CTP) of the beta subunit of human chorionic gonadotropin, polyethylene glycol (PEG), long unstructured hydrophilic sequences of amino acids (XTEN), hydroxyethyl starch (HES), an albumin-binding small molecule, and a combination thereof; and a fibronectin binding peptide.

Particular examples of fusion proteins or conjugates including IL-7 are disclosed for instance in WO19222294, WO19215510, WO19178362, WO19178364, WO19144309, WO19046313, WO18215937, WO18201047, WO18064611, WO17216223, US2018319858, WO17158436, WO16200219, WO05063820.

In a particular aspect, the IL-7 variant or mutant can be comprised in a bifunctional molecule comprising a binding moiety.

### Binding Moiety

The bifunctional molecule according to the invention comprises an IL-7 variant of mutant as disclosed herein and an additional or second entity that comprises a binding moiety.

It is understood that the binding moiety comprised in the bifunctional molecule is not an interleukin, in particular is not IL-7, nor IL-7R.

As used herein, the expression “binding moiety” relates to any moiety which have the capacity bind to a target, such as peptide, polypeptide, protein, fusion protein and antibodies. In particular, binding moieties include antibody or antigen-binding fragment thereof and antibody mimics or mimetics. Targets of binding moieties are more particularly defined hereafter.

In one embodiment, the binding moiety is selected from the group consisting of an antibody or a fragment thereof, and an antibody mimic or mimetic. Those skilled in the art of biochemistry are familiar with antibody mimics or mimetics, as discussed in Gebauer and Skerra, 2009, Curr Opin Chem Biol 13(3): 245-255. Exemplary of antibody mimics includes: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affitins (Krehenbrink et al, 2008, J. Mol. Biol. 383 (5): 1058-68), alfabodies (Desmet, J.; et al, 2014, Nature Communications. 5: 5237), fynomer (Grabulovski D; et al, 2007, J Biol Chem. 282 (5): 3196-3204) and affimers (Avacta Life Sciences, Wetherby, UK).

Accordingly, the binding moiety can be selected from the group consisting of antibody or antibody fragment thereof, preferably such as immunoglobulins, scFv or VHH, Fab, single domain antibody and antibody mimic, preferably such as affibodies, CTLDs, adnectins, anticalins, DARPins, avimers, microbodies, aptamers, Kunitz domains, affilins, affitins, alfabodies, fynomers and affimers.

Preferably, the binding moiety is an antibody or antibody fragment thereof. Even more preferably, the binding moiety is a human, humanized or chimeric antibody or antigen binding fragment thereof.

### Target of the Binding Moiety

According to the invention, the binding moiety specifically binds to a target expressed on immune cells surface, particularly targets that are only or specifically expressed on immune cells. In particular, the binding moiety is not directed towards a target expressed on tumoral cells.

With regard to the “binding” capacity of the binding moiety, the terms “bind” or “binding” refer to peptides, polypeptides, proteins, fusion proteins, molecules and antibodies (including antibody fragments and antibody mimics) that recognize and contact another peptide, polypeptide, protein or molecule. In one embodiment, it refers to an antigen-antibody type interaction. The terms “specific binding”, “specifically binds to,” “specific for,” “selectively binds” and “selective for” a particular target mean that the binding moiety recognizes and binds a specific target, but does not substantially recognize or bind other molecules in a sample. For example, an antibody that specifically (or preferentially) binds to an antigen is an antibody that binds the antigen for example with greater affinity, avidity, more readily, and/or with greater duration than it binds to other molecules. Preferably, the term “specific binding” means the contact between an antibody and an antigen with a binding affinity equal or lower than 10⁻⁷ M. In certain aspects, antibodies bind with affinities equal or lower than 10⁻⁸ M, 10⁻⁹ M or 10⁻¹⁰ M.

As used herein, the term “target” refers to a carbohydrate, lipid, peptide, polypeptide, protein, antigen or epitope that is specifically recognized or targeted by the binding moiety according to the invention and expressed on the external surface of immune cells. With regards to the expression of a target on the surface of immune cells, the term “expressed” refers to a target, such as carbohydrates, lipids, peptides, polypeptides, proteins, antigens or epitopes that are present or presented at the outer surface of a cell. The term “specifically expressed” mean that the target is expressed on immune cells, but is not substantially expressed by other cell type, particularly such as tumoral cells.

In one embodiment, the target is specifically expressed by immune cells in a healthy subject or in a subject suffering from a disease, in particular such as a cancer. This means that the target has a higher expression level in immune cells than in other cells or that the ratio of immune cells expressing the target by the total immune cells is higher than the ratio of other cells expressing the target by the total other cells. Preferably the expression level or ratio is higher by a factor 2, 5, 10, 20, 50 or 100. More specifically, it can be determined for a particular type of immune cells, for instance T cells, more specifically CD8+ T cells, effector T cells or exhausted T cells, or in a particular context, for instance a subject suffering of a disease such as a cancer or an infection.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT)) and myeloid-derived cells (neutrophil, eosinophil, basophil, monocyte, macrophage, dendritic cells). In particular, the immune cell can be selected in the non-exhaustive list comprising B cells, T cells, in particular CD4⁺ T cells and CD8⁺ T cells, NK cells, NKT cells, APC cells, macrophages, dendritic cells and monocytes.

Even more preferably, the immune cell is a T cell. “T cell” or “T lymphocytes” as used herein includes for example CD4 + T cells, CD8 + T cells, T helper 1 type T cells, T helper 2 type T cells, T regulator, T helper 17 type T cells and inhibitory T cells. In a very particular embodiment, the immune cell is an exhausted T cell.

The target can be a receptor expressed at the surface of the immune cells, especially T cells. The receptor can be an inhibitor receptor. Alternatively, the receptor can be an activating receptor.

In one aspect, the target is selected from the group consisting PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8. Such targets are more particularly described in the Table D below.

TABLE D

Example of target of interest.

| | | |
|---|---|---|
| Name | Official name | Uniprot reference |
| 2B4 | Natural killer cell receptor 2B4 (NK cell type I receptor protein 2B4, NKR2B4) (Non-MHC restricted killing associated) (SLAM family member 4, SLAMF4) (Signaling lymphocytic activation molecule 4) (CD antigen CD244) | Q07763 |
| 4-1BB | Tumor necrosis factor receptor superfamily member 9 (4-1BB ligand receptor, CD137) | Q07011 |
| BTLA | B- and T-lymphocyte attenuator (B- and T-lymphocyte-associated protein) (CD antigen CD272) | Q7Z6A9 |
| CD101 | Immunoglobulin superfamily member 2, lgSF2 (Cell surface glycoprotein V7) (Glu-Trp-lle EWI motif-containing protein 101, EWI-101) (CD antigen CD101) | Q93033 |
| CD160 | CD160 antigen (Natural killer cell receptor BY55) | O95971 |
| CD27 | CD27 antigen (CD27L receptor) (T-cell activation antigen CD27) (T14) (Tumor necrosis factor receptor superfamily member 7) (CD antigen CD27) | P26842 |
| CD28 | T-cell-specific surface glycoprotein CD28 (TP44) | P10747 |
| CD28H | Transmembrane and immunoglobulin domain-containing protein 2 (CD28 homolog) (Immunoglobulin and proline-rich receptor 1, IGPR-1) | Q96BF3 |
| CD3 | T-cell surface glycoprotein CD3 | P07766 (CD3e) P04234 (CD3d) P09693 (CD3g) |
| CD30 | Tumor necrosis factor ligand superfamily member 8 (CD30 ligand, CD30-L) (CD antigen CD153) | P32971 |
| CD38 | ADP-ribosyl cyclase/cyclic ADP-ribose hydrolase 1 (ADPRC 1, cADPr hydrolase 1) | P28907 |
| CD39 | Ectonucleoside triphosphate diphosphohydrolase-1 (NTPDase 1, Ecto-apyrase, ATPDase 1, or Lymphoid cell activation antigen) | P49961 |
| CD4 | T-cell surface glycoprotein CD4 (T-cell surface antigen T4/Leu-3) | P01730 |
| CD40L | CD40 ligand (T-cell antigen Gp39, TNF-related activation protein, Tumor necrosis factor ligand superfamily member 5, CD154) | P29965 |
| CD44 | CD44 antigen (Epican, Extracellular matrix receptor III, GP90 lymphocyte homing/adhesion receptor, HUTCH-l, Heparan sulfate proteoglycan, Hermes antigen, Hyaluronate receptor, Phagocytic glycoprotein 1, Phagocytic glycoprotein I) | P16070 |
| CD8 | T-cell surface glycoprotein CD8 | P01732 (CD8a) P10966 (CD8b) |
| CD80 | T-lymphocyte activation antigen CD80 (Activation B7-1 antigen, BB1, CTLA-4 counter-receptor B7.1, B7) | P33681 |
| CTLA-4 | Cytotoxic T-lymphocyte protein 4 (Cytotoxic T-lymphocyte-associated antigen 4, CTLA-4) (CD antigen CD152) | P16410 |
| CXCR5 | C-X-C chemokine receptor type 5 (Burkitt lymphoma receptor 1, Monocyte-derived receptor 15, CD185) | P32302 |
| DR3 | Death receptor 3 (Tumor necrosis factor receptor superfamily member 25, WSL, Apo-3, LARD) | Q93038 |
| GITR | Tumor necrosis factor receptor superfamily member 18 (Activation-inducible TNFR family receptor, Glucocorticoid-induced TNFR-related protein, CD357) | Q9Y5U5 |
| HVEM | Tumor necrosis factor receptor superfamily member 14 (Herpes virus entry mediator A, Herpesvirus entry mediator A, HveA) (Tumor necrosis factor receptor-like 2, TR2) (CD antigen CD270) | Q92956 |
| ICOS | Inducible T-cell costimulator (Activation-inducible lymphocyte immunomediatory molecule, CD278) | Q9Y6W8 |
| LAG3 | Lymphocyte activation gene 3 protein, LAG-3 (Protein FDC) (CD antigen CD223) | P18627 |
| LFA-1 | Leukocyte adhesion glycoprotein LFA-1 alpha chain (Integrin alpha-L, CD11 antigen-like family member A) | P20701 |
| NKG2D | NKG2-D type II integral membrane protein (Killer cell lectin-like receptor subfamily K member 1, NK cell receptor D, NKG2-D-activating NK receptor, CD314) | P26718 |
| OX40 | Tumor necrosis factor receptor superfamily member 4 (ACT35 antigen, AX transcriptionally-activated glycoprotein 1 receptor) | P43489 |
| PD-1 | Programmed cell death protein 1 (CD279) | Q15116 |
| PDL2 | Programmed cell death 1 ligand 2, PD-1 ligand 2, PD-L2, PDCD1 ligand 2, Programmed death ligand 2 (Butyrophilin B7-DC, B7-DC) (CD antigen CD273) | Q9BQ51 |
| SIRPG | Signal-regulatory protein gamma, SIRP-gamma (CD172 antigen-like family member B) (Signal-regulatory protein beta-2, SIRP-b2, SIRP-beta-2) (CD antigen CD172g) | Q9P1W8 |
| TIGIT | T-cell immunoreceptor with lg and ITIM domains (V-set and immunoglobulin domain-containing protein 9) (V-set and transmembrane domain-containing protein 3) | Q495A1 |
| Tim-1 | Hepatitis A virus cellular receptor 1 (T-cell immunoglobulin and mucin domain-containing protein 1, Kidney injury molecule 1, KIM-1, T-cell immunoglobulin mucin receptor 1, T-cell membrane protein 1, CD365) | Q96D42 |
| TIM3 | Hepatitis A virus cellular receptor 2, HAVcr-2 (T-cell immunoglobulin and mucin domain-containing protein 3, TIMD-3) (T-cell immunoglobulin mucin receptor 3, TIM-3) (T-cell membrane protein 3) | Q8TDQ0 |

Then, in this aspect, the binding moiety specifically binds a target selected from the group consisting PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8.

In a particular aspect, the immune cell is an exhausted T cell and the target of the binding moiety is an exhaustion factor expressed on the surface of exhausted T cells. T cell exhaustion is a state of T cell progressive loss of function, proliferation capacity and cytotoxic potential, eventually leading to their deletion. T cell exhaustion can be triggered by several factors such as persistent antigen exposure or inhibitory receptors including PD-1, TIM3, CD244, CTLA-4, LAG-3, BTLA, TIGIT and CD160. Preferably, such exhaustion factor is selected from the group consisting of PD-1, TIM3, CD244, CTLA-4, LAG-3, BTLA, TIGIT and CD160.

In a preferred embodiment, the binding moiety has an antagonist activity on the target.

Numerous antibodies directed against PD-1, TIM3, CD244, CTLA-4, LAG-3, BTLA, TIGIT and CD160 have already been described in the art.

Several anti-PD-1 are already clinically approved and others are still in clinical developments. For instance, the anti-PD1 antibody can be selected from the group consisting of Pembrolizumab (also known as Keytruda lambrolizumab, MK-3475), Nivolumab (Opdivo, MDX-1106, BMS-936558, ONO-4538), Pidilizumab (CT-011), Cemiplimab (Libtayo), Camrelizumab, AUNP12, AMP-224, AGEN-2034, BGB-A317 (Tisleizumab), PDR001 (spartalizumab), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MEDl0608, JS001 (see Si-Yang Liu et al., J. Hematol. Oncol. 10:136 (2017)), BI-754091, CBT-501, INCSHR1210 (also known as SHR-1210), TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or IBI308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168. Bifunctional or bispecific molecules targeting PD-1 are also known such as RG7769 (Roche), XmAb20717 (Xencor), MEDl5752 (AstraZeneca), FS118 (F-star), SL-279252 (Takeda) and XmAb23104 (Xencor).

In a particular embodiment, the anti-PD1 antibody can be Pembrolizumab (also known as Keytruda lambrolizumab, MK-3475) or Nivolumab (Opdivo, MDX-1106, BMS-936558, ONO-4538).

Antibodies directed against TIM3 and bifunctional or bispecific molecules targeting TIM3 are also known such as Sym023, TSR-022, MBG453, LY3321367, INCAGN02390, BGTB-A425, LY3321367, RG7769 (Roche). In some embodiments, a TFM-3 antibody is as disclosed in International Patent Application Publication Nos. WO2013006490, WO2016/161270, WO 2018/085469, or WO 2018/129553, WO 2011/155607, U.S. 8,552,156, EP 2581113 and U.S. 2014/044728.

Antibodies directed against CTLA-4 and bifunctional or bispecific molecules targeting CTLA-4 are also known such as ipilimumab, tremelimumab, MK-1308, AGEN-1884, XmAb20717 (Xencor), MEDl5752 (AstraZeneca). Anti-CTLA-4 antibodies are also disclosed in WO18025178, WO19179388, WO19179391, WO19174603, WO19148444, WO19120232, WO19056281, WO19023482, WO18209701, WO18165895, WO18160536, WO18156250, WO18106862, WO18106864, WO18068182, WO18035710, WO18025178, WO17194265, WO17106372, WO17084078, WO17087588, WO16196237, WO16130898, WO16015675, WO12120125, WO09100140 and WO07008463.

Antibodies directed against LAG-3 and bifunctional or bispecific molecules targeting LAG-3 are also known such as BMS- 986016, IMP701, MGD012 or MGD013 (bispecific PD-1 and LAG-3 antibody). Anti-LAG-3 antibodies are also disclosed in WO2008132601, EP2320940, WO19152574.

Antibodies directed against BTLA are also known in the art such as hu Mab8D5, hu Mab8A3, hu Mab21H6, hu Mab19A7, or hu Mab4C7. The antibody TAB004 against BTLA are currently under clinical trial in subjects with advanced malignancies. Anti-BTLA antibodies are also disclosed in WO08076560, WO10106051 (e.g., BTLA8.2), WO11014438 (e.g., 4C7), WO17096017 and WO17144668 (e.g., 629.3).

Antibodies directed against TIGIT are also known in the art, such as BMS-986207 or AB154, BMS-986207 CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8 as disclosed in WO19232484. Anti-TIGIT antibodies are also disclosed in WO16028656, WO16106302, WO16191643, WO17030823, WO17037707, WO17053748, WO17152088, WO18033798, WO18102536, WO18102746, WO18160704, WO18200430, WO18204363, WO19023504, WO19062832, WO19129221, WO19129261, WO19137548, WO19152574, WO19154415, WO19168382 and WO19215728.

Antibodies directed against CD160 are also known in the art, such as CL1-R2 CNCM I-3204 as disclosed in WO06015886, or others as disclosed in WO10006071, WO10084158, WO18077926.

In a preferred aspect, the binding moiety of the bifunctional molecule is an antibody, a fragment or a derivative thereof or an antibody mimic that is specific to PD-1, CTLA-4, BTLA, TIGIT, LAG3 and TIM3.

In another particular aspect, the target is PD-1 and the binding moiety of the bifunctional molecule is an antibody, a fragment or a derivative thereof or an antibody mimic that is specific to PD-1. Then, in a particular embodiment, the binding moiety comprised in the bifunctional molecule according to the invention is an anti-PD1 antibody or antigen binding fragment thereof, preferably a human, humanized or chimeric anti-PD1 antibody or antigen binding fragment thereof. Preferably, the binding moiety is an antagonist of PD-1. Therefore, the bifunctional molecule combines the effect of the IL-7 variant or mutant on the IL-7 receptor and the blockade of the inhibitory effect of PD-1, and may have a synergistic effect on the activation of T cells, especially exhausted T cells, more particularly on the TCR signaling.

In another particular aspect, the target is CTLA-4 and the binding moiety of the bifunctional molecule is an antibody, a fragment or a derivative thereof or an antibody mimic that is specific to CTLA-4. Then, in a particular embodiment, the binding moiety comprised in the bifunctional molecule according to the invention is an anti-CTLA-4 antibody or antigen binding fragment thereof, preferably a human, humanized or chimeric anti-CTLA-4 antibody or antigen binding fragment thereof. Preferably, the binding moiety is an antagonist of CTLA-4. Therefore, the bifunctional molecule combines the effect of the IL-7 variant or mutant on the IL-7 receptor and the blockade of the inhibitory effect of CTLA-4, and may have a synergistic effect on the activation of T cells, especially exhausted T cells, more particularly on the TCR signaling.

In another particular aspect, the target is BTLA and the binding moiety of the bifunctional molecule is an antibody, a fragment or a derivative thereof or an antibody mimic that is specific to BTLA. Then, in a particular embodiment, the binding moiety comprised in the bifunctional molecule according to the invention is an anti-BTLA antibody or antigen binding fragment thereof, preferably a human, humanized or chimeric anti-BTLA antibody or antigen binding fragment thereof. Preferably, the binding moiety is an antagonist of BTLA. Therefore, the bifunctional molecule combines the effect of the IL-7 variant or mutant on the IL-7 receptor and the blockade of the inhibitory effect of BTLA, and may have a synergistic effect on the activation of T cells, especially exhausted T cells, more particularly on the TCR signaling.

In another particular aspect, the target is TIGIT and the binding moiety of the bifunctional molecule is an antibody, a fragment or a derivative thereof or an antibody mimic that is specific to TIGIT. Then, in a particular embodiment, the binding moiety comprised in the bifunctional molecule according to the invention is an anti-TIGIT antibody or antigen binding fragment thereof, preferably a human, humanized or chimeric anti-TIGIT antibody or antigen binding fragment thereof. Preferably, the binding moiety is an antagonist of TIGIT. Therefore, the bifunctional molecule combines the effect of the IL-7 variant or mutant on the IL-7 receptor and the blockade of the inhibitory effect of TIGIT, and may have a synergistic effect on the activation of T cells, especially exhausted T cells, more particularly on the TCR signaling.

In another particular aspect, the target is LAG-3 and the binding moiety of the bifunctional molecule is an antibody, a fragment or a derivative thereof or an antibody mimic that is specific to LAG-3. Then, in a particular embodiment, the binding moiety comprised in the bifunctional molecule according to the invention is an anti-LAG-3 antibody or antigen binding fragment thereof, preferably a human, humanized or chimeric anti-LAG-3 antibody or antigen binding fragment thereof. Preferably, the binding moiety is an antagonist of LAG-3. Therefore, the bifunctional molecule combines the effect of the IL-7 variant or mutant on the IL-7 receptor and the blockade of the inhibitory effect of LAG-3, and may have a synergistic effect on the activation of T cells, especially exhausted T cells, more particularly on the TCR signaling.

In another particular aspect, the target is TIM3 and the binding moiety of the bifunctional molecule is an antibody, a fragment or a derivative thereof or an antibody mimic that is specific to TIM3. Then, in a particular embodiment, the binding moiety comprised in the bifunctional molecule according to the invention is an anti-TIM3 antibody or antigen binding fragment thereof, preferably a human, humanized or chimeric anti-TIM3 antibody or antigen binding fragment thereof. Preferably, the binding moiety is an antagonist of TIM3. Therefore, the bifunctional molecule combines the effect of the IL-7 variant or mutant on the IL-7 receptor and the blockade of the inhibitory effect of TIM3, and may have a synergistic effect on the activation of T cells, especially exhausted T cells, more particularly on the TCR signaling.

### Fc Domain

In a particular aspect of the present disclosure, the bifunctional molecule comprises an IL-7 variant or mutant, a binding moiety and an Fc domain. The Fc domain can be part of the binding moiety when this binding moiety is an antibody, especially an IgG immunoglobulin. However, the bifunctional molecule may have other structures including an Fc domain. For instance, it may comprise an Fc domain linked to antibody derivative such as scFv, or diabody.

One approach to improve pharmacokinetics of the bifunctional molecule according to the invention is to increase its half-life serum persistence, thereby allowing higher circulating levels, less frequent administration and reduced doses. This need can for example be met by including a Fc domain or a portion thereof in the bifunctional molecule according to the invention.

Then, in one embodiment, the bifunctional molecule according to the invention, particularly the binding moiety, comprises a Fc domain or a portion thereof.

In particular, the binding moiety according to the invention comprises at least a portion of an immunoglobulin constant region (Fc), typically that of mammalian immunoglobulin, even more preferably a chimeric, human or humanized immunoglobulin. The binding moiety can include a constant region of an immunoglobulin or a fragment, analog, variant, mutant, or derivative of the constant region. As well known by one skilled in the art, the choice of IgG isotypes of the heavy chain constant domain centers on whether specific functions are required and the need for a suitable in vivo half-life.

In preferred embodiments, the Fc domain or a fragment thereof comprised in the binding moiety comprises a heavy chain constant domain derived from a human immunoglobulin heavy chain, for example, IgG1, lgG2, lgG3, lgG4, or other classes. In a further aspect, the human constant domain is selected from the group consisting of IgG1, lgG2, lgG2, lgG3 and lgG4. Preferably, the binding moiety comprises an IgG1 or an IgG4 heavy chain constant domain.

In one embodiment, the binding moiety comprises a truncated Fc region or a fragment of the Fc region. In such Fc fragment, the constant region includes a CH2 or a CH3 domain. In another embodiment, the constant region includes CH2 and CH3 domains. Alternatively, the constant region can include all or a portion of the hinge region, the CH2 domain and/or the CH3 domain. In some embodiments, the constant region contains a CH2 and/or a CH3 domain derived from a human IgG4 or IgG1 heavy chain.

Preferably, the constant region includes all or a portion of a hinge region. The hinge region can be derived from an immunoglobulin heavy chain, e.g., IgG1, lgG2, lgG3, lgG4, or other classes. Preferably, the hinge region is derived from human IgG1, lgG2, lgG3, lgG4. More preferably the hinge region is derived from a human or humanized IgG1 or IgG4 heavy chain.

The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine.

The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8). More preferably the hinge region is derived from a human IgG4 heavy chain.

For bifunctional molecule that target cell-surface molecules, especially those on immune cells, abrogating effector functions is required. Engineering Fc regions may also be desired to either reduce or increase the effector function of the antibody.

In certain embodiments, amino acid modifications may be introduced into the Fc region to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Numerous substitutions or substitutions or deletions with altered effector function are known in the art.

In one embodiment, the constant region contains a mutation that reduces affinity for an Fc receptor or reduces Fc effector function. For example, the constant region can contain a mutation that eliminates the glycosylation site within the constant region of an IgG heavy chain. Preferably, the CH2 domain contains a mutation that eliminates the glycosylation site within the CH2 domain.

In a particular aspect, the Fc domain is modified to increase the binding to FcRn, thereby increasing the half-life of the bifunctional molecule. In another aspect or additional aspect, the Fc domain is modified to decrease the binding to FcyR, thereby reducing ADCC or CDC, or to increase the binding to FcyR, thereby increasing ADCC or CDC.

The alteration of amino acids near the junction of the Fc portion and the non-Fc portion can dramatically increase the serum half-life of the Fc fusion protein as shown in WO 01/58957. Accordingly, the junction region of a protein or polypeptide of the present invention can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life.

In one embodiment, the constant region can contain CH2 and/or CH3 has one of the mutations described in the Table E below, or any combination thereof.

TABLE E

Suitable human engineered Fc domain of an antibody. numbering of residues in the heavy chain constant region is according to EU numbering (Edelman, G.M. et al., Proc. Natl. Acad. USA, 63, 78-85 (1969); see Worldwide Website: imgt.org/IMGTScientificChart/Numbering/ Hu_IGHGnber.html#refs)

| | | | | |
|---|---|---|---|---|
| Engineered Fc | Isotype | Mutations | FcR/C1q Binding | Effector Function |
| hlgG1e1-Fc | IgG1 | T250Q/M428L | Increased binding to FcRn | Increased half-life |
| hlgG1e2-Fc | IgG1 | M252Y/S254T/T256E + H433K/N434F | Increased binding to FcRn | Increased half-life |
| hlgG1e3-Fc | IgG1 | E233P/L234V/L235A/G236A + A327G/A330S/P331S | Reduced binding to FcyRI | Reduced ADCC and CDC |
| hlgG1e4-Fc | IgG1 | E333A | Increased binding to FcyRllla | Increased ADCC and CDC |
| hlgG1e5-Fc | IgG1 | S239D/A330L/l332E | Increased binding to FcyRllla | Increased ADCC |
| hlgG1e6-Fc | IgG1 | P257I/Q311 | Increased binding to FcRn | Unchanged half-life |
| hlgG1e7-Fc | IgG1 | K326W/E333S | Increased binding to C1q | Increased CDC |
| hlgG1e9-Fc | IgG1 | S239D/I332E/G236A | Increased FcyRlla/FcyRllb ratio | Increased macrophage phagocytosis |
| hlgG1e9-Fc | IgG1 | N297A | Reduced binding to FcyRI | Reduced ADCC and CDC |
| hlgG1e9-Fc | IgG1 | LALA (L234A/L235A) | Reduced binding to FcyRI | Reduced ADCC and CDC |
| hlgG1e10-Fc | IgG1 | N297A + YTE (N298A + M252Y/S254T/T256E) | Reduced binding to FcyRI Increased binding to FcRn | Reduced ADCC and CDC Increased half-life |
| hlgG1e11-Fc | IgG1 | K322A | Reduced binding to C1q | Reduced CDC |
| hlgG1e12-Fc | IgG1 | N297A + YTE (N298A + | | Reduced ADCC and CDC |
| | | M252Y/S254T/T256E) + K444A | | Increased half-life Abolish cleavage of the C-terminal lysine of the antibody |
| hlgG4e1-Fc | IgG4 | S228P | - | Reduced Fab-arm exchange |
| hlgG4e1-Fc | IgG4 | LALA (L234A/L235A) | Increased binding to FcRn | Increased half-life |
| hlgG4e2-Fc | IgG4 | S228P+ YTE (S228P + M252Y/S254T/T256E) | - Increased binding to FcRn | Reduced Fab-arm exchange Increased half-life |
| hlgG4e3-Fc | IgG4 | N297A + YTE (N298A + M252Y/S254T/T256E) + K444A | | Reduced ADCC and CDC Increased half-life Abolish cleveage of the C-terminal lysine of the antibody |

In a particular aspect, the bifunctional molecule, preferably the binding moiety, comprises a human IgG1 heavy chain constant domain or an IgG1 Fc domain, optionally with a substitution or a combination of substitutions selected from the group consisting of T250Q/M428L; M252Y/S254T/T256E + H433K/N434F; E233P/L234V/L235A/G236A + A327G/A330S/P331S; E333A; S239D/A330L/l332E; P257l/Q311; K326W/E333S; S239D/l332E/G236A; N297A; L234A/L235A; N297A + M252Y/S254T/T256E; K322A and K444A, preferably selected from the group consisting of N297A optionally in combination with M252Y/S254T/T256E, and L234A/L235A.

In another aspect, the binding moiety comprises a human IgG4 heavy chain constant domain or a human IgG4 Fc domain, optionally with a substitution or a combination of substitutions selected from the group consisting of S228P; L234A/L235A, S228P + M252Y/S254T/T256E and K444A. Even more preferably, the bifunctional molecule, preferably the binding moiety, comprises an IgG4 Fc-region with a S228P that stabilizes the IgG4.

All subclass of Human IgG carries a C-terminal lysine residue of the antibody heavy chain (K444) that are susceptible to be cleaved off in circulation. This cleavage in the blood may compromise or decrease the bioactivity of the bifunctional molecule by releasing the linked IL-7 to IgG. To circumvent this issue, K444 amino acid in the IgG domain can be substituted by an alanine to reduce proteolytic cleavage, a mutation commonly used for antibodies. Then, in one embodiment, when the binding moiety is an antibody, the antibody comprises at least one further amino acid substitution consisting of K444A.

In one embodiment, when the binding moiety is an antibody, the antibody comprises an additional cysteine residue at the C-terminal domain of the IgG to create an additional disulfide bond and potentially restrict the flexibility of the bifunctional molecule.

In one embodiment, the binding moiety comprises an antibody. In such embodiment, such antibody has a heavy chain constant domain of SEQ ID NO: 39 or 52 and/or a light chain constant domain of SEQ ID NO: 40, particularly a heavy chain constant domain of SEQ ID NO: 39 or 52 and a light chain constant domain of SEQ ID NO: 40, particularly such as disclosed in Table F below.

In a preferred embodiment, the binding moiety comprises anti-hPD1 antibody having a heavy chain constant domain of SEQ ID NO: 52 and/or a light chain constant domain of SEQ ID. 40, particularly a heavy chain constant domain of SEQ ID NO: 52 and a light chain constant domain of SEQ ID NO: 40.

TABLE F

Example of a heavy chain constant domain and a light chain constant domain suitable for the humanized antibodies according to the invention

| | |
|---|---|
| Heavy chain constant domain (IgG4m-S228P) SEQ ID NO: 39 | ASTKGPSVFPLAPCSRSTSESTAALGCLVKDYFPEPVTVSWNSGALTSGVHTFPA VLQSSGLYSLSSVVTVPSSSLGTKTYTCNVDHKPSNTKVDKRVESKYGPPCPPCP APEFLGGPSVFLFPPKPKDTLMISRTPEVTCVVVDVSQEDPEVQFNWYVDGVE VHNAKTKPREEQFNSTYRVVSVLTVLHQDWLNGKEYKCKVSNKGLPSSIEKTISK AKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSP GK |
| Light chain constant domain (CLkappa) SEQ ID NO: 40 | RTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC |
| Heavy chain constant domain (IgG1m-N298A) SEQ ID NO: 52 | ASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKKVEPKSCDKTHTC PPCPAPELLGGPSVFLFPPKPKDTLMISRTPEVTCVVVDVSHEDPEVKFNWYVD GVEVHNAKTKPREEQYASTYRVVSVLTVLHQDWLNGKEYKCKVSNKALPAPIEK TISKAKGQPREPQVYTLPPSRDELTKNQVSLTCLVKGFYPSDIAVEWESNGQPE NNYKTTPPVLDSDGSFFLYSKLTVDKSRWQQGNVFSCSVMHEALHNHYTQKSL SLSPGK |

### Peptide Linker

In a particular aspect, the bifunctional molecule according to the invention further comprises a peptide linker connecting the binding moiety and IL-7m. The peptide linker usually has a length and flexibility enough to ensure that the IL-7m and the binding moiety connected with the linker in between have enough freedom in space to exert their functions.

In an aspect of the disclosure, the binding moiety is preferably linked to IL-7 through a peptide linker. As used herein, the term “linker” refers to a sequence of at least one amino acid that links IL-7m and the binding moiety. Such a linker may be useful to prevent steric hindrances. The linker is usually 3-44 amino acid residues in length. Preferably, the linker has 3-30 amino acid residues. In some embodiments, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

The linker sequence may be a naturally occurring sequence or a non-naturally occurring sequence. If used for therapeutic purposes, the linker is preferably non-immunogenic in the subject to which the bifunctional molecule is administered. One useful group of linker sequences are linkers derived from the hinge region of heavy chain antibodies as described in WO 96/34103 and WO 94/04678. Other examples are poly-alanine linker sequences. Further preferred examples of linker sequences are Gly/Ser linkers of different length including (Gly4Ser)₄, (Gly4Ser)₃, (Gly4Ser)₂, Gly4Ser, Gly3Ser, Gly3, Gly2ser and (Gly3Ser2)₃, in particular (Gly4Ser)₃. Preferably, the linker is selected from the group consisting of (Gly4Ser)₄, (Gly4Ser)₃, and (Gly3Ser2)₃. Even more preferably, the linker is (GGGGS)₃.

In one embodiment, the linker comprised in the bifunctional molecule is selected in the group consisting of (Gly4Ser)₄, (Gly4Ser)₃, (Gly4Ser)₂, Gly4Ser, Gly3Ser, Gly3, Gly2ser and (Gly3Ser2)₃, preferably is (Gly4Ser)₃. Preferably, the linker is selected from the group consisting of (Gly4Ser)₄, (Gly4Ser)₃, and (Gly3Ser2)₃.

### Bifunctional Molecule

The invention particularly provides a bifunctional molecule that comprises an IL-7m, a binding moiety, optionally comprising a Fc fragment, and optionally a peptide linker such as described hereabove.

In particular, the bifunctional molecule comprises or consists in a binding moiety and an IL-7m as disclosed hereabove, the binding moiety being covalently conjugated (e.g., through genetic fusion or chemical coupling) to IL-7, preferably by a peptide linker as disclosed hereabove.

In particular, the conjugation of IL-7m to the binding moiety is covalent, direct or not (i.e., via a linker), and/or chemical, enzymatic or genetic. Conjugation can be carried out by any acceptable means of bonding known in the art taking into account the chemical nature of the binding moiety. In this regard, coupling can thus be performed by one or more covalent, ionic, hydrogen, hydrophobic or Van der Waals bonds, cleavable or non-cleavable in physiological medium or within cells.

In particular, chemical conjugation can be performed through an exposed sulfhydryl group (Cys), attachment of an affinity tag (e.g. 6 Histidine, Flag Tag, Strep Tag, SpyCatcher etc.) to either the binding moiety or the IL7-m, or incorporation of unnatural amino acids or compound for click chemistry conjugation.

In a preferred embodiment, conjugation is obtained by genetic fusion (i.e., by expression in a suitable system of a nucleic acid construct encoding binding moiety and the IL-7 as a genetic fusion).

In one aspect, the invention features a fusion protein including a first portion comprising an immunoglobulin (Ig) chain, in particular a Fc domain, and a second portion comprising interleukin-7 (IL-7).

In an embodiment, the invention relates to a bifunctional molecule comprising a binding moiety fused to IL-7m. In particular, in such fusion molecule, the binding moiety is an antibody, wherein a chain of the antibody, e.g., the light or heavy chain, preferably the heavy chain, even more preferably the C-terminus of the heavy or light chain, is linked to IL-7m, preferably to the N-terminus of IL-7m, optionally by a peptide linker.

In a particular aspect, the invention relates to a bifunctional molecule comprising an antibody or antigen-binding fragment thereof and an IL-7m, wherein IL-7m is linked to the C-terminal end of the heavy chain of said antibody (e.g., the C-terminal end of the heavy chain constant domain), preferably by a peptide linker.

Preferably, the heavy chain, preferably the C terminus of the heavy chain of the antibody, is genetically fused via a flexible (Gly₄Ser)₃ linker to the N-terminus of IL-7m. At the fusion junction, the C-terminal lysine residue of the antibody heavy chain can be mutated to alanine to reduce proteolytic cleavage (i.e., mutation K444A).

In one embodiment, the bifunctional molecule according to the invention comprises one or more molecule of IL-7m. Particularly, the bifunctional molecule according to the invention may comprises one, two, three or four molecules of IL-7m. Particularly, the bifunctional molecule may comprise only one molecule of IL-7, linked to only one light chain or heavy chain of the antibody. Preferably, the bifunctional molecule may comprise only one molecule of IL-7m, preferably linked to only one heavy chain of the antibody, more preferably linked to the C-terminal end of the Fc domain of the antibody. The bifunctional molecule may also comprise two molecules of IL-7m, linked to either the light or heavy chains of the antibody. The bifunctional molecule may also comprise two molecules of IL-7m, a first one linked to the light chain of the antibody and a second one linked to the heavy chain of the antibody.

In one embodiment, the bifunctional molecule according to the invention comprises or consists of:
(a) a binding moiety that specifically binds to a target expressed on immune cells surface, such as described hereabove, conjugated to
(b) a IL-7m that presents at least 75% identity with a wild type human IL-7 (wth-IL-7) comprising or consisting of the amino acid sequence set forth in SEQ ID NO: 1, such IL-7 variant comprising at least one amino acid mutation which i) reduces affinity of the IL-7 variant for IL-7 receptor (IL-7R) in comparison to the affinity of wth-IL-7 for IL-7R, and ii) improves pharmacokinetics of the bifunctional molecule comprising the IL-7 variant in comparison with a bifunctional molecule comprising wth-IL-7.

In particular, the at least one amino acid mutation is as described hereabove under the paragraph “IL-7 mutants”.

Preferably, the bifunctional molecule according to the invention comprises or consists of:
(a) a binding moiety that specifically binds to a target expressed on immune cells surface, such as described hereabove, conjugated to
(b) a IL-7m that presents at least 75% identity with a wild type human IL-7 (wth-IL-7) comprising or consisting of the amino acid sequence set forth in SEQ ID NO: 1, such IL-7 variant comprising at least one mutation selected from the group consisting of: (i) C2S-C141S and C47S-C92S, C2S-C141S and C34S-C129S, or C47S-C92S and C34S-C129S, (ii) W142H, W142F or W142Y, (iii) D74E, D74Q or D74N, preferably D74E or D74Q; iv) Q11E, Y12F, M17L, Q22E and/or K81R; or any combination thereof.

Preferably, such mutations i) reduce affinity of the IL-7 variant for IL-7 receptor (IL-7R) in comparison to the affinity of wth-IL-7 for IL-7R, and ii) improve pharmacokinetics of the bifunctional molecule comprising the IL-7 variant in comparison with a bifunctional molecule comprising wth-IL-7. More preferably, such mutations i) reduce affinity of the IL-7 variant for IL-7 receptor (IL-7R) in comparison to the affinity of wth-IL-7 for IL-7R, ii) retain the capacity to activate IL-7R; and iii) improve pharmacokinetics of the bifunctional molecule comprising the IL-7 variant in comparison with a bifunctional molecule comprising wth-IL-7.

In a particular aspect, the target expressed on immune cells surface is an exhaustion factor expressed on T cells surface.

Preferably, the binding moiety is an antibody or an antibody fragment thereof.

Preferably, the binding moiety is conjugated to IL-7m by genetic fusion and the bifunctional molecule optionally comprises at least one peptide linker connecting the N-terminus of IL-7m to the C-terminus of the heavy chain of the antibody, the peptide linker being preferably selected from the group consisting of (GGGGS)3, (GGGGS)4, (GGGGS)2, GGGGS, GGGS, GGG, GGS and (GGGS)3, even more preferably is (GGGGS)3.

Preferably, the bifunctional molecule according to the invention is a fusion protein that comprises or consists of:
(a) an antibody or an antibody fragment thereof such as described hereabove that specifically binds to a target expressed on immune cells surface, preferably T cells,
(b) an IL-7m that presents at least 75% identity with a wild type human IL-7 (wth-IL-7) comprising or consisting of the amino acid sequence set forth in SEQ ID NO: 1, such IL-7 variant comprising the amino acids substitutions (i) C2S-C141S and C47S-C92S, C2S-C141S and C34S-C129S, or C47S-C92S and C34S-C129S, (ii) W142H, W142F or W142Y, (iii) D74E, D74Q or D74N, preferably D74E or D74Q; iv) Q11E, Y12F, M17L, Q22E and/or K81R; or any combination thereof, and
(c) optionally a peptide linker selected from the group consisting of (GGGGS)3, (GGGGS)4, (GGGGS)2, GGGGS, GGGS, GGG, GGS and (GGGS)3, preferably (GGGGS)3.

Preferably, the antibody is an antibody directed against a target selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8, preferably of PD-1, TIM3, CD244, LAG-3, BTLA, TIGIT and CD160.

Preferably, the antibody or an antibody fragment thereof has an IgG1 or IgG4 Fc domain.

In one aspect, the antibody or an antibody fragment thereof has an IgG1 Fc domain, optionally with a substitution or a combination of substitutions selected from the group consisting of K444A, T250Q/M428L; M252Y/S254T/T256E + H433K/N434F; E233P/L234V/L235A/G236A + A327G/A330S/P331S; E333A; S239D/A330L/I332E; P257I/Q311; K326W/E333S; S239D/I332E/G236A; N297A; L234A/L235A; N297A + M252Y/S254T/T256E; and K322A, preferably selected from the group consisting of N297A optionally in combination with M252Y/S254T/T256E, and L234A/L235, even more preferably an IgG1 Fc domain having the mutation N297A such as described above.

Surprisingly, the inventors observed that the bifunctional molecules having an IgG1 heavy chain constant domain have an improved activity of IL-7 variants (pStat5 signal, synergistic effect and CD127 binding) compared to the same molecule with an IgG4 heavy chain constant domain. This improvement is specific of the IL-7 mutants and has not been observed with the wildtype IL-7. In addition, the use of a long linker such as (GGGGS)₃ between the antibody and the IL-7 maximizes the activity of IL-7 variants (pStat5 signal and CD127 binding).

Accordingly, the present invention more particularly relates to a bifunctional molecule, wherein the antibody or an antibody fragment thereof such as described hereabove that specifically binds to a target expressed on immune cells surface, preferably T cells, more preferably the target being selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8, preferably of PD-1, TIM3, CD244, LAG-3, BTLA, TIGIT and CD160; and, the antibody or an antibody fragment thereof has an IgG1 Fc domain, optionally with a substitution or a combination of substitutions selected from the group consisting of T250Q/M428L; M252Y/S254T/T256E + H433K/N434F; E233P/L234V/L235A/G236A + A327G/A330S/P331S; E333A; S239D/A330L/I332E; P257I/Q311; K326W/E333S; S239D/I332E/G236A; N297A; L234A/L235A; N297A + M252Y/S254T/T256E; K322A and K444A, preferably selected from the group consisting of N297A optionally in combination with M252Y/S254T/T256E, and L234A/L235, even more preferably an IgG1 Fc domain having the mutation N297A such as described above. Preferably, the antibody or a fragment thereof is linked to IL-7 or a variant thereof by a linker selected from the group consisting of (GGGGS)₃, (GGGGS)₄, and (GGGS)₃, more preferably by (GGGGS)₃. Preferably, the IL-7 variant comprises a group of amino acid substitutions selected from the group consisting of C2S-C141S and C47S-C92S, C2S-C141S and C34S-C129S, C47S-C92S and C34S-C129S, W142H, W142F, W142Y, D74E, D74Q and D74N. More preferably, the IL-7 variant comprises a group of amino acid substitutions selected from the group consisting of C2S-C141S and C47S-C92S, C2S-C141S and C34S-C129S, W142H, W142F, W142Y, D74E, D74Q and D74N. Still more preferably, the IL-7 variant comprises a group of amino acid substitutions selected from the group consisting of C2S-C141S and C47S-C92S, C2S-C141S and C34S-C129S, W142H and D74E.

In another aspect, the antibody or an antibody fragment thereof has an IgG4 Fc domain, optionally with a substitution or a combination of substitutions selected from the group consisting of K444A, S228P; L234A/L235A, S228P + M252Y/S254T/T256E, even more preferably an IgG4 Fc domain having the mutation S228P such as described above.

In a particular aspect, the bifunctional molecule according to the invention is a fusion protein that comprises or consists of:
(a) an antibody or an antibody fragment thereof such as described hereabove that specifically binds to a target expressed on immune cells surface, preferably T cells, more preferably the target being selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8, preferably of PD-1, TIM3, CD244, LAG-3, BTLA, TIGIT and CD160;
(b) an IL-7m that presents at least 75% identity with a wild type human IL-7 (wth-IL-7) comprising or consisting of the amino acid sequence set forth in SEQ ID NO: 1, such IL-7 variant comprising the amino acids substitutions (i) C2S-C141S and C47S-C92S, C2S-C141S and C34S-C129S, or C47S-C92S and C34S-C129S, (ii) W142H, W142F or W142Y, (iii) D74E, D74Q or D74N, preferably D74E or D74Q; iv) Q11E, Y12F, M17L, Q22E and/or K81R; or any combination thereof; and
(c) optionally a peptide linker selected from the group consisting of (GGGGS)3, (GGGGS)4, (GGGGS)2, GGGS, GGG, GGS and (GGGS)3, preferably (GGGGS)3.

In a preferred embodiment of this aspect, the antibody or an antibody fragment thereof has an IgG1 Fc domain, optionally with a substitution or a combination of substitutions selected from the group consisting of T250Q/M428L; M252Y/S254T/T256E + H433K/N434F; E233P/L234V/L235A/G236A + A327G/A330S/P331S; E333A; S239D/A330L/I332E; P257I/Q311; K326W/E333S; S239D/I332E/G236A; N297A; L234A/L235A; N297A + M252Y/S254T/T256E; K322A and K444A, preferably selected from the group consisting of N297A optionally in combination with M252Y/S254T/T256E, and L234A/L235, even more preferably an IgG1 Fc domain having the mutation N297A such as described above.

Alternatively, the bifunctional molecule according to the invention is a fusion protein that comprises or consists of:
(a) an antibody or an antibody fragment thereof such as described hereabove that specifically binds to a target expressed on immune cells surface, preferably T cells; more preferably the target being selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8, preferably of PD-1, TIM3, CD244, LAG-3, BTLA, TIGIT and CD160;
(b) an IL-7m that presents at least 75% identity with a wild type human IL-7 (wth-IL-7) comprising or consisting of the amino acid sequence set forth in SEQ ID NO: 1, such IL-7 variant comprising the amino acid substitution W142H, W142F or W142Y, preferably W142H; and
(c) optionally a peptide linker selected from the group consisting of (GGGGS)3, (GGGGS)4, (GGGGS)2, GGGGS, GGGS, GGG, GGS and (GGGS)3, preferably (GGGGS)3.

Preferably, the antibody or an antibody fragment thereof has an IgG1 or IgG4 Fc domain, optionally with the substitutions as detailed above.

In a preferred embodiment of this aspect, the antibody or an antibody fragment thereof has an IgG1 Fc domain, optionally with a substitution or a combination of substitutions selected from the group consisting of T250Q/M428L; M252Y/S254T/T256E + H433K/N434F; E233P/L234V/L235A/G236A + A327G/A330S/P331S; E333A; S239D/A330L/I332E; P257I/Q311; K326W/E333S; S239D/I332E/G236A; N297A; L234A/L235A; N297A + M252Y/S254T/T256E; K322A and K444A, preferably selected from the group consisting of N297A optionally in combination with M252Y/S254T/T256E, and L234A/L235, even more preferably an IgG1 Fc domain having the mutation N297A such as described above.

Alternatively, the bifunctional molecule according to the invention comprises or consists of:
(a) an antibody or an antibody fragment thereof such as described hereabove that specifically binds to a target expressed on immune cells surface, preferably T cells; more preferably the target being selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8, preferably of PD-1, TIM3, CD244, LAG-3, BTLA, TIGIT and CD160;
(b) an IL-7m that presents at least 75% identity with a wild type human IL-7 (wth-IL-7) comprising or consisting of the amino acid sequence set forth in SEQ ID NO: 1, such IL-7 variant comprising the amino acid substitution D74E, D74Q or D74N, preferably D74E; and
(c) optionally a peptide linker selected from the group consisting of (GGGGS)3, (GGGGS)4, (GGGGS)2, GGGGS, GGGS, GGG, GGS and (GGGS)3, preferably (GGGGS)3.

Preferably, the antibody or an antibody fragment thereof has an IgG1 or IgG4 Fc domain, optionally with the substitutions as detailed above.

In a preferred embodiment of this aspect, the antibody or an antibody fragment thereof has an IgG1 Fc domain, optionally with a substitution or a combination of substitutions selected from the group consisting of T250Q/M428L; M252Y/S254T/T256E + H433K/N434F; E233P/L234V/L235A/G236A + A327G/A330S/P331S; E333A; S239D/A330L/I332E; P257I/Q311; K326W/E333S; S239D/I332E/G236A; N297A; L234A/L235A; N297A + M252Y/S254T/T256E; K322A and K444A, preferably selected from the group consisting of N297A optionally in combination with M252Y/S254T/T256E, and L234A/L235, even more preferably an IgG1 Fc domain having the mutation N297A such as described above.

Alternatively, the bifunctional molecule according to the invention comprises or consists of:
(a) an anti- PD1 antibody or antibody fragment thereof that specifically binds PD-1,
(b) an IL-7m having at least 75% identity with a wild type human IL-7 (wth-IL-7) comprising or consisting of the amino acid sequence set forth in SEQ ID NO: 1, such IL-7 variant comprising the amino acid substitution D74E, W142H and/or C2S-C141S + C47S-C92S, and
(c) optionally a peptide linker selected from the group consisting of (GGGGS)3, (GGGGS)4, (GGGGS)2, GGGGS, GGGS, GGG, GGS and (GGGS)3, preferably (GGGGS)3.

Preferably, the antibody or an antibody fragment thereof has an IgG1 or IgG4 Fc domain, optionally with the substitutions as detailed above.

Preferably, the C terminus of the heavy chain of the antibody is genetically fused via a flexible linker, preferably (Gly₄Ser)₃, to the N-terminus of IL-7m. At the fusion junction, the C-terminal lysine residue (i.e., K444) of the antibody heavy chain can be mutated to alanine to reduce proteolytic cleavage.

Optionally, the bifunctional molecule may further comprise additional moiety, such as other cytokines or other binding moieties.

In a particular aspect, the molecule has a dimeric Fc domain, on which is linked a single IL-7 variant and a single antigen-binding domain. In another particular aspect, the molecule has a dimeric Fc domain, on which is linked a single IL-7 variant and two antigen-binding domains. The antigen-binding domain binds to any target specifically expressed on immune cells surface as disclosed herein. More specifically, the target can be selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8, more specifically from the group consisting of PD-1, CTLA-4, BTLA, TIGIT, LAG3 and TIM3. In a very specific aspect, the antigen-binding domain binds to PD-1.

In a particular aspect, the molecule comprises a first monomer comprising an antigen-binding domain covalently linked to a first Fc chain optionally via a peptide linker, said first Fc chain being covalently linked to the IL-7 variant, optionally via a peptide linker, and a second monomer comprising a complementary second Fc chain, preferably devoid of antigen-binding domain and/or of an IL-7 variant, said first and second Fc chains forming a dimeric Fc domain. Optionally, the dimeric Fc domain is a heterodimeric Fc domain. More particularly, the molecule comprises a first monomer comprising an antigen-binding domain covalently linked to the N-terminal end of the first heterodimeric Fc chain optionally via a peptide linker, said first heterodimeric Fc chain being covalently linked by its C-terminal end to an IL-7 variant, optionally via a peptide linker, and a second monomer comprising a complementary second heterodimeric Fc chain devoid of antigen-binding domain. Optionally, said second monomer comprising a complementary second heterodimeric Fc chain devoid of IL-7 variant, preferably devoid of any other molecule. Optionally, said second monomer comprising a complementary second heterodimeric Fc chain covalently linked to an IL-7 variant, optionally at the N-terminal end of the C-terminal end of the Fc chain, optionally via a peptide linker. Still more particularly, the molecule comprises a first monomer comprising an antigen-binding domain covalently linked via C-terminal end to N-terminal end of a first heterodimeric Fc chain optionally via a peptide linker, said first heterodimeric Fc chain being covalently linked by its C-terminal end to the N-terminal end of the IL-7 variant, optionally via a peptide linker, and a second monomer comprising a complementary second heterodimeric Fc chain devoid of antigen-binding domain and of IL-7 variant, preferably devoid of any other molecule. Such a molecule is illustrated for example as “construct 3” in FIG. 17.

In another particular aspect, the molecule comprises a first monomer comprising an antigen-binding domain covalently linked via its C-terminal end to N-terminal end of a first heterodimeric Fc chain optionally via a peptide linker, said first heterodimeric Fc chain being covalently linked by the C-terminal end to the N-terminal end of the IL-7 variant, optionally via a peptide linker, and a second monomer comprising a complementary second heterodimeric Fc chain devoid of antigen-binding domain and covalently linked to an IL-7 variant, optionally at the N-terminal end of the C-terminal end of the Fc chain, optionally via a peptide linker. Such a molecule is illustrated for example as “construct 4” in FIG. 17.

Optionally, the complementary second heterodimeric Fc chain is covalently linked by its C-terminal end to the N-terminal end of the IL-7 variant, optionally via a peptide linker.

In an additional aspect, the molecule comprises a first monomer comprising an antigen-binding domain covalently linked to a first Fc chain, optionally via a peptide linker, said first Fc chain being optionally devoid of IL-7 variant, and a second monomer comprising a complementary second Fc chain devoid of antigen-binding domain, said second Fc chain being covalently linked to the IL-7 variant, optionally via a peptide linker, said first and second Fc chains forming a dimeric Fc domain. Optionally, the dimeric Fc domain is a heterodimeric Fc domain. More particularly, the molecule comprises a first monomer comprising an antigen-binding domain covalently linked to N-terminal end of a first heterodimeric Fc chain, optionally via a peptide linker, said first heterodimeric Fc chain being devoid of IL-7 variant, and a second monomer comprising a complementary second heterodimeric Fc chain devoid of antigen-binding domain, said second heterodimeric Fc chain being covalently linked by C-terminal end to the IL-7 variant, optionally via a peptide linker. Still more particularly, the molecule comprises a first monomer comprising an antigen-binding domain covalently linked by C-terminal end to N-terminal end of a first heterodimeric Fc chain, optionally via a peptide linker, said first heterodimeric Fc chain being devoid of IL-7 variant, and a second monomer comprising a complementary second heterodimeric Fc chain devoid of antigen-binding domain, said second heterodimeric Fc chain being covalently linked by C-terminal end to N-terminal of the IL-7 variant, optionally via a peptide linker.

In another particular aspect, the molecule comprises a first monomer comprising an antigen-binding domain covalently linked to a first Fc chain optionally via a peptide linker, said first Fc chain being covalently linked to the IL-7 variant, optionally via a peptide linker, and a second monomer comprising a complementary second Fc chain devoid of IL-7 variant and being linked to an antigen-binding domain, said first and second Fc chains forming a dimeric Fc domain. Such a molecule is illustrated for example as “construct 2” in FIG. 17. Optionally, the dimeric Fc domain is a heterodimeric Fc domain. More particularly, the molecule comprises a first monomer comprising an antigen-binding domain covalently linked to the N-terminal end of the first heterodimeric Fc chain optionally via a peptide linker, said first heterodimeric Fc chain being covalently linked by its C-terminal end to an IL-7 variant, optionally via a peptide linker, and a second monomer comprising a complementary second heterodimeric Fc chain devoid of IL-7 variant and comprising an antigen-binding domain covalently linked to the N-terminal end of the second heterodimeric Fc chain optionally via a peptide linker. More particularly, the molecule comprises a first monomer comprising an antigen-binding domain covalently linked via C-terminal end to N-terminal end of a first heterodimeric Fc chain optionally via a peptide linker, said first heterodimeric Fc chain being covalently linked by its C-terminal end to the N-terminal end of the IL-7 variant, optionally via a peptide linker, and a second monomer comprising a complementary second heterodimeric Fc chain devoid of IL-7 variant and comprising an antigen-binding domain covalently linked via C-terminal end to N-terminal end of said second heterodimeric Fc chain optionally via a peptide linker.

The linker, if present, can be selected among the linkers disclosed herein.

Preferably, two monomers comprise each one a Fc chain, the Fc chains being able to form a dimeric Fc domain.

In one aspect, the dimeric Fc fusion protein is a homodimeric Fc fusion protein. In another aspect, the dimeric Fc fusion protein is a heterodimeric Fc fusion protein.

More specifically, the Fc domain is a heterodimeric Fc domain. Heterodimeric Fc domains are made by altering the amino acid sequence of each monomer. The heterodimeric Fc domains rely on amino acid variants in the constant regions that are different on each chain to promote heterodimeric formation and/or allow for ease of purification of heterodimers over the homodimers. There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”. Heterodimerization variants can include steric variants (e.g. the “knobs and holes” or “skew” variants described below and the “charge pairs” variants described below) as well as “pi variants”, which allows purification of homodimers away from heterodimers. WO2014/145806, hereby incorporated by reference in its entirety, discloses useful mechanisms for heterodimerization include “knobs and holes”, “electrostatic steering” or “charge pairs”, pi variants, and general additional Fc variants. See also, Ridgway et al., Protein Engineering 9(7):617 (1996); Atwell et al., J. Mol. Biol. 1997 270:26; U.S. Pat. No. 8,216,805, Merchant et al., Nature Biotech. 16:677 (1998), all of which are hereby incorporated by reference in their entirety. For “electrostatic steering” see Gunasekaran et al., J. Biol. Chem. 285(25): 19637 (2010), hereby incorporated by reference in its entirety. For pi variants, see US 2012/0149876 hereby incorporated by reference in its entirety.

Then, in a preferred aspect, the heterodimeric Fc domain comprises a first Fc chain and a complementary second Fc chain based on the “knobs and holes” technology. For instance, the first Fc chain is a “knob” or K chain, meaning that it comprises the substitution characterizing a knob chain, and the second Fc chain is a “hole” or H chain, meaning that it comprises the substitution characterizing a hole chain. And vice versa, the first Fc chain is a “hole” or H chain, meaning that it comprises the substitution characterizing a hole chain, and the second Fc chain is a “knob” or K chain, meaning that it comprises the substitution characterizing a knob chain. In a preferred aspect, the first Fc chain is a “hole” or H chain and the second Fc chain is a “knob” or K chain.

Examples of bifunctional molecules structures according to the invention are provided FIG. 17.

Optionally, the heterodimeric Fc domain may comprise one heterodimeric Fc chain which comprises the substitutions as shown in the following table and the other heterodimeric Fc chain comprising the substitutions as shown in the following table.

TABLE G

(the numbering being according to EU index)

| | |
|---|---|
| Fc chain having the following substitutions (Hole chain or H chain) | The complementary Fc chain having the following substitutions (Knob chain or K chain) |
| D221E/P228E/L368E | D221R/P228R/K409R |
| C220E/P228E/368E | C220R/E224R/P228R/K409R |
| S364K/E357Q | L368D/K370S |
| L368D/K370S | S364K |
| L368E/K370S | S364K |
| T411T/E360E/Q362E | D401K |
| L368D/K370S | S364K/E357L |
| K370S | S364K/E357Q |
| T366S/L368A/Y407V | T366W |
| T366S/L368A/Y407V/Y349C | T366W/S354C |
| F368D/K370S | S364K |
| F368D/K370S | S364K/E357F |
| F368D/K370S | S364K/E357Q |
| T411E/K360E/Q362E | D401K |
| F368E/K370S | S364K |
| K370S | S364K/E357Q |
| T366S/F368A/Y407V | T366W |
| T366S/L368A/Y407V/Y349C | T366W/S354C |

In a preferred aspect, the first Fc chain is a “hole” or H chain and comprises the substitutions T366S/L368A/Y407V/Y349C and the second Fc chain is a “knob” or K chain and comprises the substitutions T366W/S354C.

Optionally, the Fc chain may further comprise additional substitutions.

In one aspect, the bifunctional molecule according to the invention comprises a heterodimer of Fc domains that comprises the “knob into holes” modifications such as described above. Preferably, such Fc domains are IgG1 or IgG4 Fc domain such as described above, even more preferably an IgG1 Fc domain comprising the mutation N297A such as disclosed above.

For instance, the first Fc chain is a “hole” or H chain and comprises the substitutions T366S/L368A/Y407V/Y349C and N297A and the second Fc chain is a “knob” or K chain and comprises the substitutions T366W/S354C and N297A. More particularly, the second Fc chain may comprise or consists in SEQ ID NO: 75 and/or the first Fc chain may comprise or consists in SEQ ID NO: 77.

More specifically, the IL7 variant according to the invention is linked to the knob-chain and/or the hole chain of the heterodimeric Fc domain. Thus, the bifunctional molecule according to the invention may comprises i) a single IL7 variant either linked to the hole-chain or to the knob-chain of the Fc domain, or ii) two IL7 variants, one linked to hole-chain and one linked to the knob-chain of the Fc domain. Preferably, the bifunctional molecule according to the invention comprises a single IL7 variant linked to the hole-chain of the Fc domain.

In a first aspect, the bifunctional molecule comprises an IL7 variant linked to the C-terminal or the N-terminal of the knob-chain Fc domain. Optionally, such Fc domain is not linked to an antigen binding domain. Alternatively, such Fc domain is linked to an antigen binding domain.

In a second aspect, the bifunctional molecule comprises an IL7 variant linked to the C-terminal of the hole-chain Fc domain. Preferably, such Fc-domain is linked to an antigen binding domain at its N-terminal end.

Optionally, the bifunctional molecule comprises a single IL7 variant linked to the C-terminal of the hole-chain of the Fc domain, wherein the bifunctional molecule only comprises a single antigen binding domain linked in the N-terminal end of the hole chain of the Fc domain. In such aspect, the knob chain domain is devoid of an IL7 variant and is or not devoid of an antigen binding domain.

More particularly, the bifunctional molecule comprises a single IL7 variant linked to the C-terminal end of the hole-chain of the Fc domain preferably by its N terminal end, optionally by a linker, wherein the bifunctional molecule only comprises a single antigen binding domain linked at the N-terminal end of the hole chain of the Fc domain, and a knob chain devoid of IL7 variant and of antigen binding domain.

Accordingly, an object of the present invention relates to a polypeptide comprising from the N-terminal to the C-terminal an antigen binding domain (or at least the part therefor corresponding to the heavy chain), a Fc chain (knob or hole Fc chain), preferably the hole-chain of the Fc domain, and an IL7 variant. The complementary chain comprises a complementary Fc chain devoid of IL7 variant and antigen binding domain, preferably the knob-chain of the Fc domain.

In another particular aspect, the bifunctional molecule comprises a single IL7 variant linked to the C-terminal end of the hole-chain of the Fc domain by its N terminal end, optionally by a linker, wherein the bifunctional molecule comprises an antigen binding domain linked at the N-terminal end of the hole chain of the Fc domain, and a knob chain devoid of IL7 variant and comprising an antigen binding domain linked to the N-terminal end of the knob chain by its C-terminal end.

Accordingly, an object of the present invention relates to a polypeptide comprising from the N-terminal to the C-terminal an antigen binding domain (or at least the part therefor corresponding to the heavy chain), a Fc chain (knob or hole Fc chain), preferably the hole-chain of the Fc domain, and an IL7 variant. The complementary chain comprises from the N-termina to the C-terminal an antigen binding domain (or at least the part therefor corresponding to the heavy chain) and a complementary Fc chain devoid of IL7 variant, preferably the knob-chain of the Fc domain.

In another particular aspect, the bifunctional molecule comprises a single IL7 variant linked to the N- or C-terminal end of the knob chain, optionally by a linker, and the bifunctional molecule comprises an antigen binding domain linked at the N-terminal end of the hole chain of the Fc domain by its C-terminal end, the hole chain being devoid of IL7 variant.

Optionally, the antigen-binding domain can be a Fab domain, a Fab′, a single-chain variable fragment (scFV) or a single domain antibody (sdAb). The antigen-binding domain preferably comprises a heavy chain variable region (VH) and a light chain variable region (VL). When the antigen-binding domain is a Fab or a Fab′, the molecule further comprises a heavy chain and a light chain constant domain (i.e. CH and CL).

When the antigen binding domain is a Fab or a Fab′, the bifunctional molecule may further comprise an IL-7 variant linked to the C terminal of the VL domain of the antigen-binding domain.

The bifunctional molecule according to the invention may comprise one or two antigen binding domains. Optionally, one antigen binding domain can be linked to the N-terminal of the knob Fc chain and one antigen binding domain can be linked to the N-terminal of the hole Fc chain. Alternatively, a single antigen binding domain is linked to the N-terminal of either the knob Fc chain or the hole Fc chain. Preferably, the IL-7 variant is linked to the Fc chain linked to the antigen binding domain. In a particular aspect, the antigen-binding domain targets PD-1.

For instance, the antigen-binding domain targeting PD-1 can be derived from an anti-PD1 antibody selected from the group consisting of Pembrolizumab (also known as Keytruda lambrolizumab, MK-3475), Nivolumab (Opdivo, MDX-1106, BMS-936558, ONO-4538), Pidilizumab (CT-011), Cemiplimab (Libtayo), Camrelizumab, AUNP12, AMP-224, AGEN-2034, BGB-A317 (Tisleizumab), PDR001 (spartalizumab), MK-3477, SCH-900475, PF-06801591, JNJ-63723283, genolimzumab (CBT-501), LZM-009, BCD-100, SHR-1201, BAT-1306, AK-103 (HX-008), MEDI-0680 (also known as AMP-514) MEDI0608, JS001 (see Si-Yang Liu et al., J. Hematol. Oncol.10:136 (2017)), BI-754091, CBT-501, INCSHR1210 (also known as SHR-1210), TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or IBI308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168. Bifunctional or bispecific molecules targeting PD-1 are also known such as RG7769 (Roche), XmAb20717 (Xencor), MEDI5752 (AstraZeneca), FS118 (F-star), SL-279252 (Takeda) and XmAb23104 (Xencor). In particular, the antigen-binding domain targeting PD-1 comprises the 6 CDRs or the VH and VL of an anti-PD1 antibody selected in this list. Such antigen-binding domain can particularly be a Fab or svFc domain derived from this antibody. In a preferred aspect, the antigen-binding domain targeting PD-1 comprises the 6 CDRs or the VH and VL of the anti-PD1 antibody selected from Pembrolizumab (also known as Keytruda lambrolizumab, MK-3475) or Nivolumab (Opdivo, MDX-1106, BMS-936558, ONO-4538) and can be for instance a Fab or a scFc domain.

In a specific aspect, the antigen-binding domain targeting PD-1 is derived from the antibody disclosed in WO2020/127366, the disclosure thereof being incorporated herein by reference.

Then, the antigen-binding domain comprises:
(i) a heavy chain variable domain comprising HCDR1, HCDR2 and HCDR3, and
(ii) a light chain variable domain comprising LCDR1, LCDR2 and LCDR3,
wherein:
the heavy chain CDR1 (HCDR1) comprises or consists of an amino acid sequence of SEQ ID NO: 51, optionally with one, two or three modification(s) selected from substitution(s), addition(s), deletion(s) and any combination thereof at any position but position 3 of SEQ ID NO: 51;
the heavy chain CDR2 (HCDR2) comprises or consists of an amino acid sequence of SEQ ID NO: 53, optionally with one, two or three modification(s) selected from substitution(s), addition(s), deletion(s) and any combination thereof at any position but positions 13, 14 and 16 of SEQ ID NO: 53;
the heavy chain CDR3 (HCDR3) comprises or consists of an amino acid sequence of SEQ ID NO: 54 wherein X1 is D or E and X2 is selected from the group consisting of T, H, A, Y, N, E and S, preferably in the group consisting of H, A, Y, N, E; optionally with one, two or three modification(s) selected from substitution(s), addition(s), deletion(s) and any combination thereof at any position but positions 2, 3, 7 and 8 of SEQ ID NO: 54;
the light chain CDR1 (LCDR1) comprises or consists of an amino acid sequence of SEQ ID NO: 63 wherein X is G or T, optionally with one, two or three modification(s) selected from substitution(s), addition(s), deletion(s) and any combination thereof at any position but positions 5, 6, 10, 11 and 16 of SEQ ID NO: 63;
the light chain CDR2 (LCDR2) comprises or consists of an amino acid sequence of SEQ ID NO: 66, optionally with one, two or three modification(s) selected from substitution(s), addition(s), deletion(s) and any combination thereof; and
the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16.

In one aspect, the antigen-binding domain comprises:
(i) a heavy chain variable domain comprising HCDR1, HCDR2 and HCDR3, and
(ii) a light chain variable domain comprising LCDR1, LCDR2 and LCDR3, wherein:
   the heavy chain CDR1 (HCDR1) comprises or consists of an amino acid sequence of SEQ ID NO: 51, optionally with one, two or three modification(s) selected from substitution(s), addition(s), deletion(s) and any combination thereof at any position but position 3 of SEQ ID NO: 51;
   the heavy chain CDR2 (HCDR2) comprises or consists of an amino acid sequence of SEQ ID NO: 53, optionally with one, two or three modification(s) selected from substitution(s), addition(s), deletion(s) and any combination thereof at any position but positions 13, 14 and 16 of SEQ ID NO: 53;
   the heavy chain CDR3 (HCDR3) comprises or consists of an amino acid sequence of SEQ ID NO: 54 wherein either X1 is D and X2 is selected from the group consisting of T, H, A, Y, N, E, and S preferably in the group consisting of H, A, Y, N, E; or X1 is E and X2 is selected from the group consisting of T, H, A, Y, N, E and S, preferably in the group consisting of H, A, Y, N, E and S; optionally with one, two or three modification(s) selected from substitution(s), addition(s), deletion(s) and any combination thereof at any position but positions 2, 3, 7 and 8 of SEQ ID NO: 54;
   the light chain CDR1 (LCDR1) comprises or consists of an amino acid sequence of SEQ ID NO: 63 wherein X is G or T, optionally with one, two or three modification(s) selected from substitution(s), addition(s), deletion(s) and any combination thereof at any position but positions 5, 6, 10, 11 and 16 of SEQ ID NO: 63;
   the light chain CDR2 (LCDR2) comprises or consists of an amino acid sequence of SEQ ID NO: 66, optionally with one, two or three modification(s) selected from substitution(s), addition(s), deletion(s) and any combination thereof; and
   the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16.

In another embodiment, the antigen-binding domain comprises or consists essentially of: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or SEQ ID NO: 65, a CDR2 of SEQ ID NO: 66 and a CDR3 of SEQ ID NO: 16.

In another aspect, the antigen-binding domain comprises or consists essentially of:
(i) a heavy chain comprising a CDR1 of SEQ ID NO: 51, a CDR2 of SEQ ID NO: 53 and a CDR3 of SEQ ID NO: 55; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 56; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 57; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 58; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 59; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 60; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 61; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 62; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 55; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 56; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 57; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 58; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 59; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 60; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 61; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 62; and (ii) a light chain comprising a CDR1 of SEQ ID NO: 65, a CDR2 of SEQ ID NO: 66 and a CDR3 of SEQ ID NO: 16.

In one aspect, the anti-PD1 antibody or antigen binding fragment according to the invention comprises framework regions, in particular heavy chain variable region framework regions (HFR) HFR1, HFR2, HFR3 and HFR4 and light chain variable region framework regions (LFR) LFR1, LFR2, LFR3 and LFR4.

Preferably, the anti-PD1 antibody or antigen binding fragment according to the invention comprises human or humanized framework regions. A “human acceptor framework” for the purposes herein is a framework comprising the amino acid sequence of a light chain variable domain (VL) framework or a heavy chain variable domain (VH) framework derived from a human immunoglobulin framework or a human consensus framework, as defined below. A human acceptor framework derived from a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence. A “human consensus framework” is a framework which represents the most commonly occurring amino acid residues in a selection of human immunoglobulin VL or VH framework sequences.

Particularly, the anti-PD1 antibody or antigen binding fragment comprises heavy chain variable region framework regions (HFR) HFR1, HFR2, HFR3 and HFR4 comprising an amino acid sequence of SEQ ID NOs: 41, 42, 43 and 44, respectively, optionally with one, two or three modification(s) selected from substitution(s), addition(s), deletion(s) and any combination thereof at any position but positions 27, 29 and 32 of HFR3, i.e., of SEQ ID NO: 43. Preferably, the anti-PD1 antibody or antigen binding fragment comprises HFR1 of SEQ ID NO: 41, HFR2 of SEQ ID NO: 42, HFR3 of SEQ ID NO: 43 and HFR4 of SEQ ID NO: 44.

Alternatively or additionally, the anti-PD1 antibody or antigen binding fragment comprises light chain variable region framework regions (LFR) LFR1, LFR2, LFR3 and LFR4 comprising an amino acid sequence of SEQ ID NOs: 45, 46, 47 and 48, respectively, optionally with one, two or three modification(s) selected from substitution(s), addition(s), deletion(s) and any combination thereof. Preferably, the humanized anti-PD1 antibody or antigen binding fragment comprises LFR1 of SEQ ID NO: 45, LFR2 of SEQ ID NO: 46, LFR3 of SEQ ID NO: 47 and LFR4 of SEQ ID NO: 48.

The VL and VH domain of the anti hPD1 antibody comprised in the bifunctional molecule according to the invention may comprise four framework regions interrupted by three complementary determining regions preferably operably linked in the following order: FR1-CDR1-FR2-CDR2-FR3-CDR3-FR4 (from amino terminus to carboxy terminus).

In an aspect, the antigen-binding domain comprises or consists essentially of:
(a) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In another aspect, the antigen-binding domain comprises or consists essentially of:
(a) a heavy chain variable region (VH) comprising or consisting of an amino acid sequence of SEQ ID NO: 18, 19, 20, 21, 22, 23, 24 or 25, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18, 19, 20, 21, 22, 23, 24 or 25 respectively;
(b) a light chain variable region (VL) comprising or consisting of an amino acid sequence of SEQ ID NO: 27 or SEQ ID NO: 28, optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: 27 or SEQ ID NO: 28.

In another aspect, the antigen-binding domain comprises or consists essentially of:
(a) a heavy chain variable region (VH) comprising or consisting of an amino acid sequence of SEQ ID NO: 18, 19, 20, 21, 22, 23, 24 or 25;
(b) a light chain variable region (VL) comprising or consisting of an amino acid sequence of SEQ ID NO: 27 or SEQ ID NO: 28.

In another aspect, the antigen-binding domain comprises or consists essentially of any of the following combinations of a heavy chain variable region (VH) and a light chain variable region (VL):

| VH (SEQ ID NO:), optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: | VL (SEQ ID NO:), optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: |
|---|---|
| 18 | 27 |
| 18 | 28 |
| 19 | 27 |
| 19 | 28 |
| 20 | 27 |
| 20 | 28 |
| 21 | 27 |
| 21 | 28 |
| 22 | 27 |
| 22 | 28 |
| 23 | 27 |
| 23 | 28 |
| 24 | 27 |
| 24 | 28 |
| 25 | 27 |
| 25 | 28 |

In very particular aspect, the antigen-binding domain comprises or consists essentially of a heavy chain variable region (VH) of SEQ ID NO: 24 and a light chain variable region (VL) of SEQ ID NO: 28.

In a particular embodiment, the bifunctional molecule comprises:
(a) a heavy chain comprising or consisting of an amino acid sequence selected from the group consisting of SEQ ID NO: 29, 30, 31, 32, 33, 34, 35 or 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, 30, 31, 32, 33, 34, 35 or 36, respectively, and the substitutions corresponding to the hole or knob chain, preferably the hole chain, more specifically as disclosed in Table G, in particular, in SEQ ID NO: 29, 30, 31, 32, 33, 34, 35 or 36, either T363S/L365A/Y4047V/Y346C or T363W/S351C, preferably T363S/L365A/Y4047V/Y346C, and optionally N294A in any of SEQ ID NO: 29, 30, 31, 32, 33, 34, 35 or 36;
(b) a light chain comprising or consisting of an amino acid sequence of SEQ ID NO: 37 or SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37 or SEQ ID NO: 38.

In another aspect, the bifunctional molecule comprises or consists in any of the following combinations of a heavy chain (CH) and a light chain (CL):

| CH (SEQ ID NO:), optionally with one, two or three modification(s) selected from substitution(s), addition(s), deletion(s) at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, 30, 31, 32, 33, 34, 35 or 36 of SEQ ID NO: | CL (SEQ ID NO:), optionally with one, two or three modification(s) selected from substitution(s), addition(s), deletion(s) at any position but positions 3, 4, 7, 14, 17, 18, 28, 29, 33, 34, 39, 42, 44, 50, 81, 88, 94, 97, 99 and 105 of SEQ ID NO: |
|---|---|
| 27 | 37 |
| 27 | 38 |
| 28 | 37 |
| 28 | 38 |
| 29 | 37 |
| 29 | 38 |
| 30 | 37 |
| 30 | 38 |
| 31 | 37 |
| 31 | 38 |
| 32 | 37 |
| 32 | 38 |
| 33 | 37 |
| 33 | 38 |
| 34 | 37 |
| 34 | 38 |
| 35 | 37 |
| 35 | 38 |
| 36 | 37 |
| 36 | 38 |

with the heavy chain comprising the substitutions corresponding to the hole or knob chain, preferably the hole chain, more specifically as disclosed in Table G, in particular, in SEQ ID NO: 29, 30, 31, 32, 33, 34, 35 or 36, in particular either T366S/L368A/Y407V/Y349C or T366W/S354C, preferably T366S/L368A/Y407V/Y349C, and optionally N297A in any of SEQ ID NO: 29, 30, 31, 32, 33, 34, 35 or 36, the positions of the substitutions being defined according to EU numbering.

Accordingly, in one aspect, the bifunctional molecule according to the invention comprises or consists of:
(a) an anti-human PD-1 antigen-binding domain, which comprises (i) one heavy chain with a first Fc chain, and (ii) one light chain,
(b) an IL-7 variant, and
(c) a complementary second Fc chain,
wherein the IL-7 variant is covalently linked, optionally via a peptide linker, preferably by its N-terminal end to the C-terminal end of the first Fc chain and/or to the N- or C-terminal end of the second Fc chain.

The IL-7 variant can be any IL-7 variant as disclosed above.

The first and second Fc chain can be as disclosed above. Preferably, the Fc chains are preferably Fc chains from an IgG1 or a IgG4 antibody.

The anti-human PD-1 antigen-binding domain is as disclosed above.

In one aspect, the bifunctional molecule comprises a single anti-human PD-1 antigen-binding domain (only one). Preferably, the bifunctional molecule comprises a single anti-human PD-1 antigen-binding domain selected from the group consisting of an anti-human PD-1 Fab, an anti-human PD-1 Fab′, an anti-human PD-1 scFV and an anti-human PD-1 sdAb.

The bifunctional molecule comprises one or two IL-7 variants, preferably a single IL-7 variant.

The bifunctional molecule may comprise a light chain comprising or consisting of SEQ ID NO: 37 or 38.

The bifunctional molecule may comprise a heavy chain comprising or consisting of any of the SEQ ID NOs: 29, 30, 31, 32, 33, 34, 35 and 36, the Fc chain being optionally modified to promote a heterodimerization of the Fc chains for forming a heterodimeric Fc domain. More specifically, the heavy chain comprises the substitutions corresponding to the hole or knob chain, preferably the hole chain, more specifically as disclosed in Table G, particularly either T366S/L368A/Y407V/Y349C or T366W/S354C, preferably T366S/L368A/Y407V/Y349C, and optionally N297A in any of SEQ ID NO: 29, 30, 31, 32, 33, 34, 35 or 36, the positions of the substitutions being defined according to EU numbering.

In a very particular aspect, the bifunctional molecule comprises a light chain comprising or consisting of SEQ ID NO: 38 and a heavy chain comprising or consisting of SEQ ID NO: 35, the Fc chain being optionally modified to promote a heterodimerization of the Fc chains for forming a heterodimeric Fc domain.

In a very particular aspect, the bifunctional molecule may comprise a first monomer of SEQ ID NO: 75 and a second monomer comprising a Fc chain SEQ ID NO: 77, to which is linked at the N-terminal end, optionally by a linker, to an antigen binding domain (in particular of SEQ ID NO: 79), and at the C-terminal end, optionally by a linker, to any IL-7 variant as disclosed herein. More particularly, the bifunctional molecule comprises a first monomer of SEQ ID NO: 75, a second monomer of SEQ ID NO: 83, and a third monomer of SEQ ID NO: 37 38 or 80, preferably SEQ ID NO: 38 or 80.

In another very particular aspect, the bifunctional molecule may comprise a first monomer of SEQ ID NO: 77 and a second monomer comprising a Fc chain SEQ ID NO: 75, to which is linked at the N-terminal end, optionally by a linker, to an antigen binding domain (in particular of SEQ ID NO: 79), and at the C-terminal end, optionally by a linker, to any IL-7 variant as disclosed herein. More particularly, the bifunctional molecule comprises a first monomer of SEQ ID NO: 77, a second monomer of SEQ ID NO: 82, and a third monomer of SEQ ID NO: 37 38 or 80, preferably SEQ ID NO: 38 or 80.

In another very particular aspect, the bifunctional molecule may comprise a first monomer of SEQ ID NO: 75 to which is linked at the N-terminal end, optionally by a linker, to an antigen binding domain (in particular of SEQ ID NO: 79), and a second monomer comprising a Fc chain SEQ ID NO: 77, to which is linked at the N-terminal end, optionally by a linker, to an antigen binding domain (in particular of SEQ ID NO: 79), and at the C-terminal end, optionally by a linker, to any IL-7 variant as disclosed herein. More particularly, the bifunctional molecule comprises a first monomer of SEQ ID NO: 81, a second monomer of SEQ ID NO: 83, and a third monomer of SEQ ID NO: 37, 38 or 80, preferably SEQ ID NO: 38 or 80.

In another very particular aspect, the bifunctional molecule may comprise a first monomer of SEQ ID NO: 77 to which is linked at the N-terminal end, optionally by a linker, to an antigen binding domain (in particular of SEQ ID NO: 79), and a second monomer comprising a Fc chain SEQ ID NO: 75, to which is linked at the N-terminal end, optionally by a linker, to an antigen binding domain (in particular of SEQ ID NO: 79), and at the C-terminal end, optionally by a linker, to any IL-7 variant as disclosed herein.

### Preparation of Bifunctional Molecule - Nucleic Acid Molecules Encoding the IL-7 Variants or Mutants, or the Fusion Proteins and Bifunctional Molecules Comprising Them, Recombinant Expression Vectors and Host Cells Comprising Such

To produce an IL-7 variant or mutant, a fusion protein or a bifunctional molecule according to the invention, in particular by mammalian cells, nucleic acid sequences or group of nucleic acid sequences coding for the IL-7 variant or mutant, the fusion protein or the bifunctional molecule are subcloned into one or more expression vectors. Such vectors are generally used to transfect mammalian cells. General techniques for producing molecules comprising antibody sequences are described in Coligan et al. (eds.), Current protocols in immunology, at pp. 10.19.1-10.19.11 (Wiley Interscience 1992), the contents of which are hereby incorporated by reference and in “Antibody engineering: a practical guide” from W. H. Freeman and Company (1992), in which commentary relevant to production of molecules is dispersed throughout the respective texts.

Generally, such method comprises the following steps of:
(1) transfecting or transforming appropriate host cells with the polynucleotide(s) or its variants encoding the IL-7 variant or mutant, a fusion protein or recombinant bifunctional molecule of the invention or the vector containing the polynucleotide(s);
(2) culturing the host cells in an appropriate medium; and
(3) optionally isolating or purifying the protein from the medium or host cells.

The invention further relates to a nucleic acid encoding an IL-7 variant or mutant, a fusion protein or bifunctional molecule as disclosed above, a vector, preferably an expression vector, comprising the nucleic acid of the invention, a genetically engineered host cell transformed with the vector of the invention or directly with the sequence encoding the IL-7 variant or mutant, the fusion protein or the recombinant bifunctional molecule, and a method for producing the protein of the invention by recombinant techniques.

The nucleic acid, the vector and the host cells are more particularly described hereafter.

### Nucleic Acid Sequence

The invention also relates to a nucleic acid molecule encoding the IL-7 variant or mutant, the fusion protein or the bifunctional molecule as defined above or to a group of nucleic acid molecules encoding the IL-7 variant or mutant, the fusion protein or the bifunctional molecule as defined above. Nucleic acid encoding the IL-7 variant or mutant, the fusion protein or the bifunctional molecule disclosed herein can be amplified by any techniques known in the art, such as PCR. Such nucleic acid may be readily isolated and sequenced using conventional procedures.

Particularly, the nucleic acid molecules encoding the bifunctional molecule as defined herein comprises:
a first nucleic acid molecule encoding a binding moiety as disclosed herein, and
a second nucleic acid molecule encoding IL-7m, preferably a human IL-7m.

In a very particular embodiment, the nucleic acid molecule encoding the binding moiety comprises a variable heavy chain domain having the sequence set forth in SEQ ID NO: 73 and/or a variable light chain domain having the sequence set forth in SEQ ID NO: 74.

In one embodiment, the second nucleic acid molecule is operably linked to the first nucleic acid, optionally through a nucleic acid encoding a peptide linker. By operably linked is intended that the nucleic acid encodes a protein fusion. Then, in a particular aspect, the nucleic acid encodes a fusion protein including the binding moiety, optionally the peptide linker, and the IL-7 variant disclosed herein. Preferably, in such nucleic acid molecule, when the binding moiety comprises a Fc domain, the N-terminal of the IL-7 variant is fused to the C-terminal of the heavy chain constant domain, preferably via a peptide linker.

In one embodiment, the nucleic acid molecule is an isolated, particularly non-natural, nucleic acid molecule.

In one aspect, the nucleic acid encodes the IL-7m having the amino acid sequence set forth in SEQ ID NOs: 2 to 15.

### Vectors

In another aspect, the invention relates to a vector comprising the nucleic acid molecule or the group of nucleic acid molecules as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The nucleic acid molecule encoding the bifunctional molecule, the fusion protein, the binding moiety or the IL-7 variant can be cloned into a vector by those skilled in the art, and then transformed into host cells. These methods include in vitro recombinant DNA techniques, DNA synthesis techniques, in vivo recombinant techniques, etc. The methods known to the artisans in the art can be used to construct an expression vector containing the nucleic acid sequence of the bifunctional molecule, the fusion protein, the binding moiety or the IL-7 variant described herein and appropriate regulatory components for transcription/translation.

Accordingly, the present invention also provides a recombinant vector, which comprises a nucleic acid molecule encoding the bifunctional molecule, the fusion protein, the binding moiety or the IL-7 variant according to the present invention. In one preferred embodiment, the expression vector further comprises a promoter and a nucleic acid sequence encoding a secretion signal peptide, and optionally at least one drug-resistance gene for screening. The expression vector may further comprise a ribosome -binding site for initiating the translation, transcription terminator and the like.

Suitable expression vectors typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants.

### Host Cells

In another aspect, the invention relates to a host cell comprising a vector or a nucleic acid molecule or group of nucleic acid molecules as defined above, for example for bifunctional molecule production purposes.

As used herein, the term “host cell” is intended to include any individual cell or cell culture that can be or has been recipient of vectors, exogenous nucleic acid molecules, and polynucleotides encoding the bifunctional molecule, the fusion protein, the binding moiety or the IL-7 variant according to the present invention. The term “host cell” is also intended to include progeny or potential progeny of a single cell. Suitable host cells include prokaryotic or eukaryotic cells, and also include but are not limited to bacteria, yeast cells, fungi cells, plant cells, and animal cells such as insect cells and mammalian cells, e.g., murine, rat, rabbit, macaque or human.

Suitable hosts cells are especially eukaryotic hosts cells which provide suitable post-translational modifications such as glycosylation. Preferably, such suitable eukaryotic host cell may be fungi such as Pichia pastoris, Saccharomyces cerevisiae, Schizosaccharomyces pombe; insect cell such as Mythimna separate; plant cell such as tobacco, and mammalian cells such as BHK cells, 293 cells, CHO cells, NSO cells and COS cells.

Preferably, the host cell of the present invention is selected from the group consisting of CHO cell, COS cell, NSO cell, and HEK cell.

Then host cells stably or transiently express the bifunctional molecule, the fusion protein, the binding moiety and/or the IL-7 variant according to the present invention. Such expression methods are known by the man skilled in the art.

A method of production of the IL-7 variant or mutant, the fusion protein or the bifunctional molecule is also provided herein. The method comprises culturing a host cell comprising a nucleic acid encoding the bifunctional molecule, the fusion protein, the binding moiety and/or the IL-7 variant, as provided above, under conditions suitable for its expression, and optionally recovering the bifunctional molecule, the fusion protein, the binding moiety and/or the IL-7 variant from the host cell (or host cell culture medium). Particularly, for recombinant production of a bifunctional molecule, nucleic acid encoding a bifunctional molecule, e.g., as described above, is isolated and inserted into one or more vectors for further cloning and/or expression in a host cell. The IL-7 variants or mutants, the fusion proteins bifunctional molecules are then isolated and/or purified by any methods known in the art. These methods include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, any other liquid chromatography, and the combination thereof. As described, for example, by Coligan, bifunctional molecule isolation techniques may particularly include affinity chromatography with Protein-A Sepharose, size-exclusion chromatography and ion exchange chromatography. Protein A preferably is used to isolate the bifunctional molecules of the invention.

### Pharmaceutical Composition and Method of Administration Thereof

The present invention also relates to a pharmaceutical composition comprising any of the IL-7 variants or mutants, the fusion proteins or the bifunctional molecules described herein, the nucleic acid molecule, the group of nucleic acid molecules, the vector and/or the host cells as described hereabove, preferably as the active ingredient or compound. The formulations can be sterilized and, if desired, mixed with auxiliary agents such as pharmaceutically acceptable carriers, excipients, salts, anti-oxidant and/or stabilizers which do not deleteriously interact with the bifunctional molecule of the invention, nucleic acid, vector and/or host cell of the invention and does not impart any undesired toxicological effects. Optionally, the pharmaceutical composition may further comprise an additional therapeutic agent.

Particularly, the pharmaceutical composition according to the invention can be formulated for any conventional route of administration including a topical, enteral, oral, parenteral, intranasal, intravenous, intramuscular, subcutaneous or intraocular administration and the like. To facilitate administration, the bifunctional molecule as described herein can be made into a pharmaceutical composition for in vivo administration. The means of making such a composition have been described in the art (see, for instance, Remington: The Science and Practice of Pharmacy, Lippincott Williams & Wilkins, 21st edition (2005).

The pharmaceutical composition may be prepared by mixing a bifunctional molecule having the desired degree of purity with optional pharmaceutically acceptable carriers, excipients, anti-oxidant, and/or stabilizers in the form of lyophilized formulations or aqueous solutions. Such suitable carriers, excipients, anti-oxidant, and/or stabilizers are well known in the art and have been for example described in Remington’s Pharmaceutical Sciences 16th edition, Osol, A. Ed. (1980).

To facilitate delivery, any of the bifunctional molecule or its encoding nucleic acids can be conjugated with a chaperon agent. The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polypeptide.

Pharmaceutical compositions according to the invention may be formulated to release the active ingredients (e.g. the bifunctional molecule of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition. Such systems can avoid repeated administrations of the composition, thereby increasing convenience to the subject and the physician.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer.

Pharmaceutical composition typically must be sterile and stable under the conditions of manufacture and storage. Prevention of presence of microorganisms may be ensured both by sterilization procedures (for example by microfiltration), and/or by the inclusion of various antibacterial and antifungal agents.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect.

### Subject, Regimen and Administration

The present invention relates to an IL-7 variant or mutant, a fusion protein or a bifunctional molecule as disclosed herein; a nucleic acid or a vector encoding such, a host cell or a pharmaceutical composition, a nucleic acid, a vector or a host cell, for use as a medicament or for use in the treatment of a disease or for administration in a subject or for use as a medicament. It also relates to a method for treating a disease or a disorder in a subject comprising administering a therapeutically effective amount of a pharmaceutical composition or a bifunctional molecule to a subject. Examples of treatments are more particularly described hereafter under the section “Methods and Uses”.

The subject to treat may be a human, particularly a human at the prenatal stage, a new-born, a child, an infant, an adolescent or an adult, in particular an adult of at least 30 years old, 40 years old, preferably an adult of at least 50 years old, still more preferably an adult of at least 60 years old, even more preferably an adult of at least 70 years old.

In a particular aspect, the subject can be immunosuppressed or immunocompromised.

Conventional methods, known to those of ordinary skill in the art of medicine, can be used to administer the bifunctional molecule or the pharmaceutical composition disclosed herein to a subject, depending upon the type of diseases to be treated or the site of the disease e.g., administered orally, parenterally, enterally, by inhalation spray, topically, rectally, nasally, buccally, vaginally or via an implanted reservoir. Preferably, the bifunctional molecule or the pharmaceutical composition is administered via subcutaneous, intra-cutaneous, intravenous, intramuscular, intra-articular, intraarterial, intra-synovial, intra-tumoral, intra-sternal, intra-thecal, intra-lesion, and intracranial injection or infusion techniques.

The form of the pharmaceutical compositions, the route of administration and the dose of administration of the pharmaceutical composition or the bifunctional molecule according to the invention can be adjusted by the man skilled in the art according to the type and severity of the infection, and to the patient, in particular its age, weight, size, sex, and/or general physical condition. The compositions of the present invention may be administered in a number of ways depending upon whether local or systemic treatment is desired.

### Use in the Treatment of a Disease

The bifunctional molecules, nucleic acids, vectors, host cells, compositions and methods of the present invention have numerous in vitro and in vivo utilities and applications. Particularly, any of the IL-7 variants or mutants, fusion proteins or bifunctional molecules, nucleic acid molecules, group of nucleic acid molecules, vectors, host cells or pharmaceutical composition provided herein may be used in therapeutic methods and/or for therapeutic purposes.

The present invention also relates to an IL-7 variant or mutant, a fusion protein or a bifunctional molecule, a nucleic acid or a vector encoding such, or a pharmaceutical composition comprising such for use in the treatment of a disorder and/or disease in a subject and/or for use as a medicament or vaccine. It also relates to the use of an IL-7 variant or mutant, a fusion protein or a bifunctional molecule as described herein; a nucleic acid or a vector encoding such, or a pharmaceutical composition comprising such for treating a disease and/or disorder in a subject. Finally, it relates to a method for treating a disease or a disorder in a subject comprising administering a therapeutically effective amount of a pharmaceutical composition or an IL-7 variant or mutant, a fusion protein or a bifunctional molecule to the subject, or a nucleic acid or a vector encoding such.

In one embodiment, the invention relates to a method of treatment of a disease and/or disorder selected from the group consisting of a cancer, an infectious disease and a chronic viral infection in a subject in need thereof comprising administering to said subject an effective amount of the IL-7 variant or mutant, fusion protein or bifunctional molecule or pharmaceutical composition as defined above. Examples of such diseases are more particularly described hereafter.

In one aspect, the treatment method comprises: (a) identifying a patient in need of treatment; and (b) administering to the patient a therapeutically effective amount of any of the IL-7 variant or mutant, fusion protein or bifunctional molecule, nucleic acid, vector or pharmaceutical composition described herein.

A subject in need of a treatment may be a human having, at risk for, or suspected of having a disease. Such a patient can be identified by routine medical examination.

In another aspect, the bifunctional molecules disclosed herein can be administered to a subject, e.g., in vivo, to enhance immunity, preferably in order to treat a disorder and/or disease. Accordingly, in one aspect, the invention provides a method of modifying an immune response in a subject comprising administering to the subject a bifunctional molecule, nucleic acid, vector or pharmaceutical composition of the invention such that the immune response in the subject is modified. Preferably, the immune response is enhanced, increased, stimulated or up-regulated. The bifunctional molecule or pharmaceutical composition can be used to enhance immune responses such as T cell activation in a subject in need of a treatment. In a particular embodiment, the bifunctional molecule or pharmaceutical composition can be used to reduce T cells exhaustion or to reactivate exhausted T cells.

The invention particularly provides a method of enhancing an immune response in a subject, comprising administering to the subject a therapeutic effective amount of any of the bifunctional molecule, nucleic acid, vector or pharmaceutical composition comprising such described herein, such that an immune response in the subject is enhanced. In a particular embodiment, the bifunctional molecule or pharmaceutical composition can be used to reduce T cells exhaustion or to reactivate exhausted T cells.

Bifunctional molecules according to the invention target CD127+ immune cells, particularly CD127+ T cells. Such cells may be found in the following areas of particular interest: resident lymphoid cells in the lymph nodes (mainly within paracortex, with occasional cells in follicles), in tonsil (inter-follicular areas), spleen (mainly within the Peri-Arteriolar Lymphoid Sheaths (PALS) of the white pulp and some scattered cells in the red pulp), thymus (primarily in medulla; also in cortex), bone marrow (scattered distribution), in the GALT (Gut Associated-Lymphoid-Tissue, primarily in inter-follicular areas and lamina propria) throughout the digestive tract (stomach, duodenum, jejunum, ileum, cecum colon, rectum), in the MALT (Mucosa-Associated-Lymphoid-Tissue) of the gall bladder. Therefore, the bifunctional molecules of the invention are of particular interest for treating diseases located or involving these areas, in particular cancers.

### Cancer

In another embodiment, the invention provides the use of an IL-7 variant or mutant, a fusion protein or a bifunctional molecule or pharmaceutical composition as disclosed herein in the manufacture of a medicament for treating a cancer, for instance for inhibiting growth of tumor cells in a subject.

The term “cancer” as used herein is defined as disease characterized by the rapid and uncontrolled growth of aberrant cells. Cancer cells can spread locally or through the bloodstream and lymphatic system to other parts of the body.

Accordingly, in one embodiment, the invention provides a method of treating a cancer, for instance for inhibiting growth of tumor cells, in a subject, comprising administering to the subject a therapeutically effective amount of bifunctional molecule or pharmaceutical composition according to the invention. Particularly, the present invention relates to the treatment of a subject using a bifunctional molecule such that growth of cancerous cells is inhibited.

In an aspect of the disclosure, the cancer to be treated is associated with exhausted T cells.

Any suitable cancer may be treated with the provided herein can be hematopoietic cancer or solid cancer. Such cancers include carcinoma, cervical cancer, colorectal cancer, esophageal cancer, gastric cancer, gastrointestinal cancer, head and neck cancer, kidney cancer, liver cancer, lung cancer, lymphoma, glioma, mesothelioma, melanoma, stomach cancer, urethral cancer environmentally induced cancers and any combinations of said cancers. Additionally, the invention includes refractory or recurrent malignancies. Preferably, the cancer to be treated or prevented is selected from the group consisting of metastatic or not metastatic, Melanoma, malignant mesothelioma, Non-Small Cell Lung Cancer, Renal Cell Carcinoma, Hodgkin’s Lymphoma, Head and Neck Cancer, Urothelial Carcinoma, Colorectal Cancer, Hepatocellular Carcinoma, Small Cell Lung Cancer Metastatic Merkel Cell Carcinoma, Gastric or Gastroesophageal cancers and Cervical Cancer.

In a particular aspect, the cancer is a hematologic malignancy or a solid tumor. Such a cancer can be selected from the group consisting of hematolymphoid neoplasms, angioimmunoblastic T cell lymphoma, myelodysplasic syndrome, acute myeloid leukemia.

In a particular aspect, the cancer is a cancer induced by virus or associated with immunodeficiency. Such a cancer can be selected from the group consisting of Kaposi sarcoma (e.g., associated with Kaposi sarcoma herpes virus); cervical, anal, penile and vulvar squamous cell cancer and oropharyndeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection.

Preferred cancers for treatment include cancers typically responsive to immunotherapy. Alternatively, preferred cancers for treatment are cancers non-responsive to immunotherapy.

### Infectious Disease

The bifunctional molecule, nucleic acid, group of nucleic acid, vector, host cells or pharmaceutical compositions of the invention can be used to treat patients that have been exposed to particular toxins or pathogens. Accordingly, an aspect of the invention provides a method of treating an infectious disease in a subject comprising administering to the subject a bifunctional molecule according to the present invention, or a pharmaceutical composition comprising such, preferably such that the subject is treated for the infectious disease.

Any suitable infection may be treated with a bifunctional molecule, nucleic acid, group of nucleic acid, vector, host cells or pharmaceutical composition as provided herein.

Some examples of pathogenic viruses causing infections treatable by methods of the invention include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods of the invention include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

### Combined Therapy

The bifunctional molecule according to the invention can be combined with some other potential strategies for overcoming immune evasion mechanisms with agents in clinical development or already on the market (see table 1 from Antonia et al. Immuno-oncology combinations: a review of clinical experience and future prospects. Clin. Cancer Res. Off. J. Am. Assoc. Cancer Res. 20, 6258-6268, 2014). Such combination with the bifunctional molecule according to the invention may be useful notably for:
1- Reversing the inhibition of adaptive immunity (blocking T-cell checkpoint pathways);
2- Switching on adaptive immunity (promoting T-cell costimulatory receptor signaling using agonist molecules, in particular antibodies);
3- Improving the function of innate immune cells;
4- Activating the immune system (potentiating immune-cell effector function), for example through vaccine-based strategies.

Accordingly, also provided herein are combined therapies with any of the bifunctional molecule or pharmaceutical composition comprising such, as described herein and a suitable second agent, for the treatment of a disease or disorder. In an aspect, the bifunctional molecule and the second agent can be present in a unique pharmaceutical composition as described above. Alternatively, the terms “combination therapy” or “combined therapy”, as used herein, embrace administration of these two agents (e.g., a bifunctional molecule as described herein and an additional or second suitable therapeutic agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route. The agents can be administered by the same route or by different routes. For example, a first agent (e.g., a bifunctional molecule) can be administered orally, and an additional therapeutic agent (e.g., an anti-cancer agent, an anti-infection agent; or an immune modulator) can be administered intravenously. Alternatively, an agent of the combination selected may be administered by intravenous injection while the other agents of the combination may be administered orally.

In an aspect, the additional therapeutic agent can be selected in the non-exhaustive list comprising alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton’s tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

For instance, the additional therapeutic agent can be selected in the group consisting of chemotherapy, radiotherapy, targeted therapy, antiangiogenic agents, hypomethylating agents, cancer vaccines, epitopes or neoepitopes from tumor antigens, myeloid checkpoints inhibitors, other immunotherapies, and HDAC inhibitors.

The present invention also relates to a method for treating a disease in a subject comprising administering to said subject a therapeutically effective amount of the bifunctional molecule or the pharmaceutical composition described herein and a therapeutically effective amount of an additional or second therapeutic agent.

Specific examples of additional or second therapeutic agents are provided in WO 2018/053106, pages 36-43.

In a preferred embodiment, the second therapeutic agent is selected from the group consisting of chemotherapeutic agents, radiotherapy agents, immunotherapeutic agents, cell therapy agents (such as CAR-T cells), antibiotics and probiotics.

Combination therapy could also rely on the combination of the administration of bifunctional molecule with surgery.

### Kits

Any of the bifunctional molecules or compositions described herein may be included in a kit provided by the present invention. The present disclosure particularly provides kits for use in enhancing immune responses and/or treating diseases or disorders (e.g. cancer and/or infection) .

In the context of the present invention, the term “kit” means two or more components (one of which corresponding to the bifunctional molecule, the nucleic acid molecule, the vector or the cell of the invention) packaged in a container, recipient or otherwise. A kit can hence be described as a set of products and/or utensils that are sufficient to achieve a certain goal, which can be marketed as a single unit. The kits of this invention are in suitable packaging.

Particularly, a kit according to the invention may comprise:
an IL-7 variant or mutant, a fusion protein or a bifunctional molecule as defined above,
a nucleic acid molecule or a group of nucleic acid molecules encoding said IL-7 variant or mutant, fusion protein or bifunctional molecule,
a vector comprising said nucleic acid molecule or group of nucleic acid molecules, and/or
a cell comprising said vector or nucleic acid molecule or group of nucleic acid molecules.

The kit may thus include, in suitable container means, the pharmaceutical composition, and/or the IL-7 variants or mutants, fusion proteins or bifunctional molecules, and/or host cells of the present invention, and/or vectors encoding the nucleic acid molecules of the present invention, and/or nucleic acid molecules or related reagents of the present invention. In some embodiments, means of taking a sample from an individual and/or of assaying the sample may be provided. The compositions comprised in the kit according to the invention may particularly be formulated into a syringe compatible composition.

In some embodiments, the kit further includes an additional agent for treating cancer or an infectious disease, and the additional agent may be combined with the IL-7 variant or mutant, fusion protein or bifunctional molecule, or other components of the kit of the present invention or may be provided separately in the kit. Particularly, the kit described herein may include one or more additional therapeutic agents such as those described in the “Combined Therapy” described hereabove. The kit(s) may be tailored to a particular cancer for an individual and comprise respective second cancer therapies for the individual as described hereabove.

The instructions related to the use of the bifunctional molecule or pharmaceutical composition described herein generally include information as to dosage, dosing schedule, route of administration for the intended treatment, means for reconstituting the bifunctional molecule and/or means for diluting the bifunctional molecule of the invention. Instructions supplied in the kits of the invention are typically written instructions on a label or package insert (e.g., a paper sheet included in the kit in the form of a leaflet or instruction manual).

### EXAMPLES

### Example 1. Mutations of Fc Fused IL-7 Modify Binding to IL-7R and pSTAT5 Signaling and Improves Pharmacokinetics In Vivo

To obtain IL-7 mutants, amino-acids implicated in the interaction IL7 to CD127 were substituted with amino-acid possessing similar nature and properties. Several mutants were generated, namely Q11E, Y12F, M17L, Q22E, D74E, D74Q, D74N, K81R, W142H, W142F and W142Y.

IL-7 disulfide bonds were disrupted by replacing cysteine residues by serine residues, leading to the substitution C2S-C141S + C34S-C129S (mutant named “SS1”), or C2S-C141S + C47S-C92S (mutant named “SS2”), or C47S-C92S + C34S-C129S (mutant named “SS3”).

TABLE 1

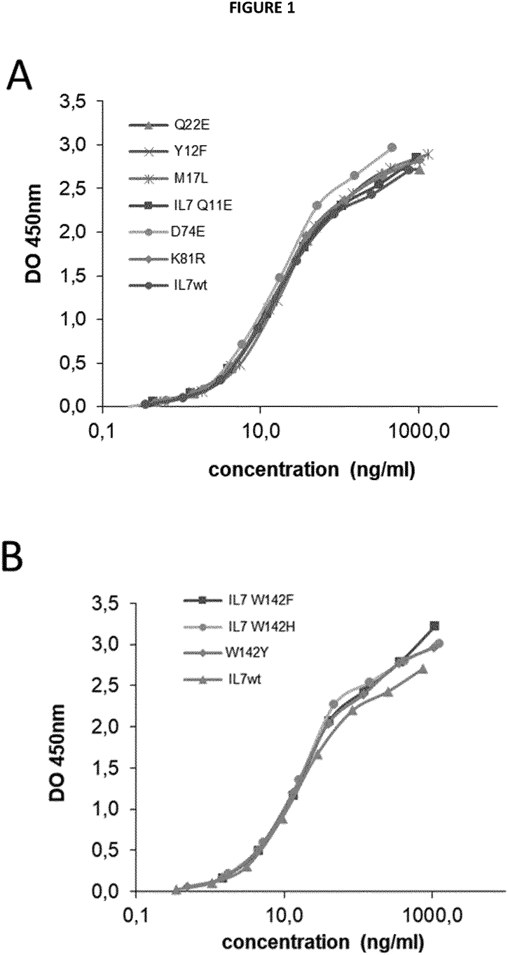

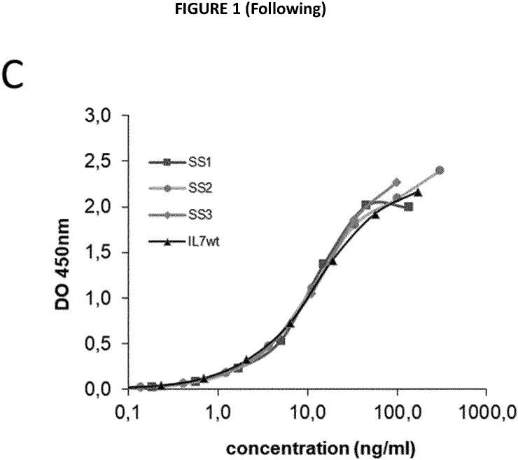

ED50 determination from FIGS. 1A, B and C refers to the concentration required to reach 50% of the binding to CD127 receptor. Each table represent a different experiment and can be compared to the positive control IgG4 G4S3 IL7WT

| Samples | EC50 ng/mL |
|---|---|
| IgG4 G4S3 IL7 WT | 18.4 |
| IgG4 G4S3 IL7 Q11E | 18.49 |
| IgG4 G4S3 IL7 Y12F | 22.27 |
| IGG4 G4S3 IL7 M17L | 20.96 |
| IGG4 G4S3 IL7 Q22E | 17.44 |
| IgG4 G4S3 IL7 D74E | 103.94 |
| IgG4 G4S3 IL7 K81R | 20.18 |
| IgG4 IL7 G4S3 W142F | 34.86 |
| IgG4 G4S3 IL7 W142H | 136.32 |
| IGG4 G4S3 IL7 W142Y | 44.6 |

TABLE 2

Binding of WT versus mutated IL-7 to CD127 receptor. Affinity assessment by Biacore of fused anti PD-1 IL-7 for CD127. A two-state reaction model was used for analysis

| Samples | Ka (⅟Ms) | Kd2 (⅟s) | KD (M) |
|---|---|---|---|
| IgG4 Fc G4S3 IL-7 WT | 5.76E+06 | 1.22E-04 | 4.14E-11 |
| IgG4 Fc G4S3 IL-7 W142H | 5.02E+05 | 2.56E-03 | 5.68E-08 |
| IgG4 Fc G4S3 Fc IL-7 SS2 | 6.11E+05 | 1.55E-03 | 7.22E-09 |
| IgG4 Fc G4S3 Fc IL-7 SS3 | 1962 | 6.02E-4 | 1.36E-6 |

TABLE 3

Binding of WT versus mutated IL-7 to CD132 receptor. Affinity assessment by Biacore of the complex CD127 + IgG fused IL-7 on CD132. A steady-state reaction model was used for analysis

| | |
|---|---|
| Samples | KD CD132 |
| IgG4 alone | 2.50E-06 |
| IgG4 G4S3 IL-7 WT | 1.18E-07 |
| IgG4 Fc G4S3 IL-7 W142H | 5.72E-07 |
| IgG4 Fc G4S3 Fc IL-7 SS2 | 3.10E-06 |

TABLE 4

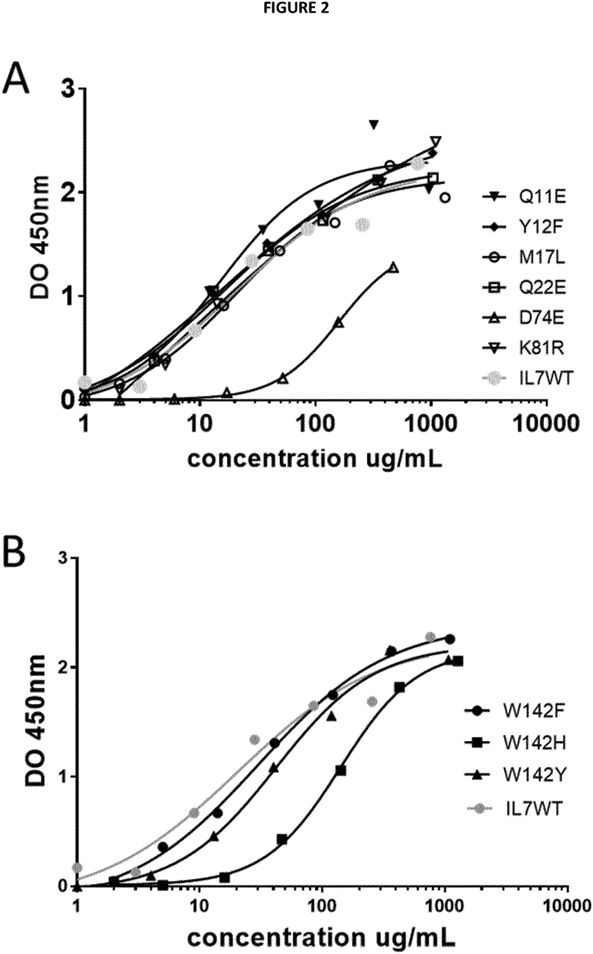

ED50 determination from FIGS. 2A, B and C refers to the concentration required to reach 50% of the pSTAT5 signal in this assay for each anti PD-1 IL-7 molecule. Each table represents a different experiment with a different donor and each table can be compared to the positive control IgG4 G4S3 IL7WT

| Samples | EC50 ng/mL | | |
|---|---|---|---|
| IgG4 G4S3 IL7 WT | 76 | | |
| IgG4 IL7 Q11E | 77 | | |
| IgG4 G4S3 IL7 Y12F | 66 | | |
| IGG4 G4S3 IL7 M17L | 128 | | |
| IGG4 G4S3 IL7 Q22E | 84 | | |
| IgG4 G4S3 IL7D74E | 389 | | |
| IgG4 G4S3 IL7 K81R | 79 | Samples | EC50 ng/mL |
| IgG4 G4S3 IL7 W142F | 102 | IgG4 G4S3 IL7 WT | 0.52 |
| IgG4 G4S3 IL7 W142H | 861 | IgG4 G4S3 IL7 SS2 | 2401 |
| IgG4 G4S3 IL7 W142Y | 208 | IGG4 G4S3 IL7 SS3 | 4348 |

TABLE 5

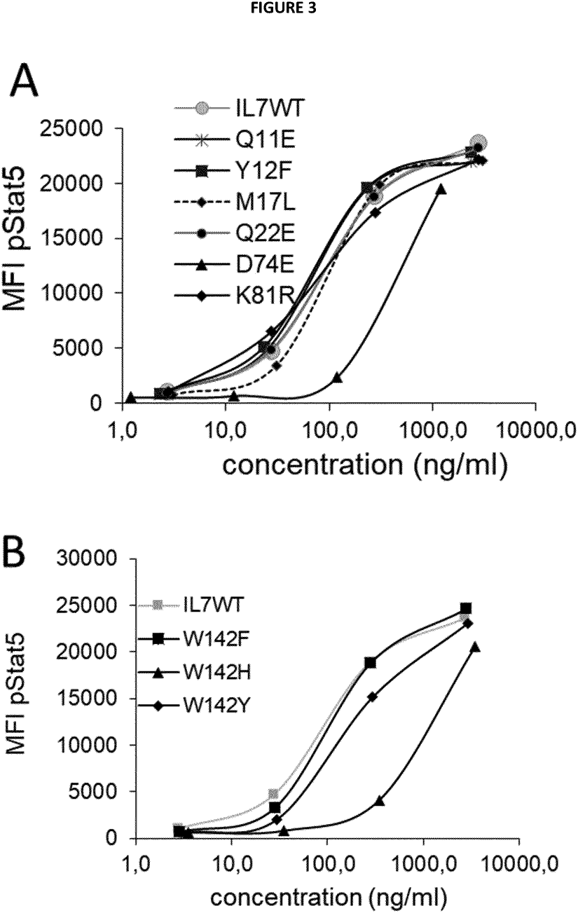

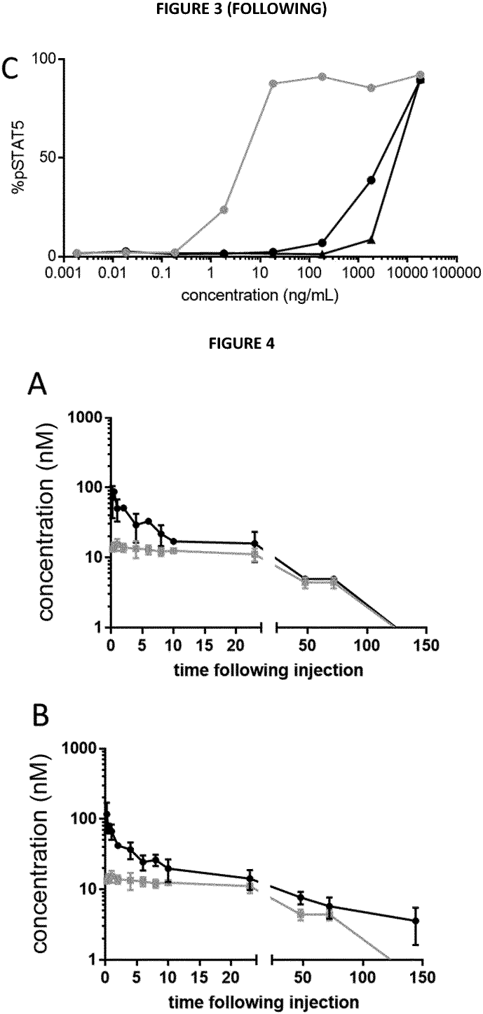

Cmax, area under the curve and half-life determination from FIG. 3 . Cmax was calculated at the time point 15 minutes following anti PD-1 IL7 injection. AUC was calculated from 0 to 144 hours following injection of the anti PD-1 IL-7

| Samples | C max obtained (nM) | Area under curve (AUC) |
|---|---|---|
| IgG4 G4S3 IL7 WT | 13.22 | 121.4 |
| IgG4 G4S3 IL7D74E | 89.19 | 151.9 |
| IgG4 G4S3 IL7 W142F | 98 | Undetermined |
| IgG4 G4S3 IL7 W142H | 141 | 248.2 |
| IgG4 G4S3 IL7 W142Y | 70 | Undetermined |
| IgG4 G4S3 SS2 | 69.9 | 361.6 |
| IgG4 G4S3 SS3 | 140.6 | 466.5 |

The substitution of one amino-acid in IL7 sequence did not modify its capacity to bind PD-1 receptor (FIGS. 1 A, B and C ). However, these mutations modify its biological activity as shown by CD127 binding and pSTAT5 signaling in ex vivo T cells assay (FIGS. 2 and 3 and Tables 1 and 4). The mutation D74E and W142H are the most efficient mutation to decrease both IL-7 binding to CD127 and activation of pStat5 in T lymphocytes (FIGS. 2A, 2B and 3A, 3B and Table 1 and 5). In another experiment, the effect of disulfilde bounds disruption was analyzed (FIG. 2C). At high concentration (10 µg/ml), SS2 or SS3 were able to activate pStat5 in T lymphocyte, with 3log deviation from IL-7 WT (FIG. 2C and Table 4).

To confirm the binding capacity of those mutants, a Biacore assay was performed to determine the KD (equilibrium dissociation constant between the receptor and its antigen, see Table 2). Mutants SS2 and W142H have a lower affinity to CD127 with a KD close to 7 to 57 nM. The SS3 mutant has the lowest affinity for the CD127 with a KD close to 3 µM. The affinity for the CD132 receptor was also assessed as shown on Table 3. In this experiment, IgG4 alone was used as baseline KD affinity as CD127 dimerizes with CD132 in the absence of IL-7. IL-7 mutant W142H binds to CD132 but with 5-fold higher affinity compared to the IgG IL-7WT. This data demonstrates that the mutation W142H decreases binding to CD127 and redirect binding of IL-7 toward the CD132 receptor, leading to a loss of pSTAT5 activation in T cells as shown on FIG. 2. In contrast, the inventors observed in the condition tested that SS2 mutant loses the capacity to bind to CD132 receptor, suggesting that the SS2 mutant preferentially binds to CD127 over CD132 receptor, leading to a decrease pSTAT5 activity in T cells (FIG. 3).

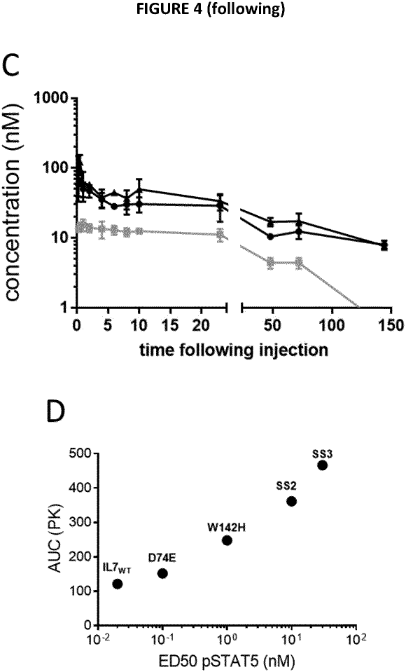

To determine pharmacokinetics/pharmacodynamics of the anti PD-1 IL-7 in vivo, mice were intravenously injected with one dose of IgG-IL-7 (34.4 nM/kg). Plasma drug concentration was analyzed by ELISA specific for human IgG. FIG. 3 and Table 5 show that IgG4 IL-7 WT molecules have rapid distribution as the Cmax (maximal concentration 15 minutes following injection) obtained is 30-fold lower than theorical concentration. All the W142Y, F, H mutants tested depicted a better distribution profile with a Cmax 5 to 10-fold higher than the IL-7 WT (FIG. 3A and Table 5). The W142H mutant presents the best Cmax. Anti PD-1 IL-7 D74E mutant also demonstrated a good Cmax. The mutants SS2 and SS3 exhibit the best PK profile with a 7 to 13-fold higher Cmax than IL-7 WT and good linear profile curve. In parallel, the AUC (Area under the curve) was determined (Table 5 and FIG. 4D), the AUC gives insight into the extent of drug exposure and its clearance rate from the body. These data demonstrate that the AUC increased with the IL-7 mutants meaning that the IL-7 mutants have an improved drug exposure. As represented in FIG. 4D, the inventors observed that the drug exposure correlates with the IL-7 potency of the mutant (measured by pSTAT5 EC50). In conclusion, the affinity of IL-7 is correlated with the pharmacokinetics of the product. Decreasing affinity of IL-7 to their receptors CD127 and CD132 improves the absorption and distribution of the IL-7 bifunctional molecules in vivo.

### Example 2: The Addition of a Cysteine at the C-Terminal Domain at the C-Terminal Domain of the IgG Decreases the Flexibility of the IL7 Molecule and Improve Pharmacokinetics In Vivo

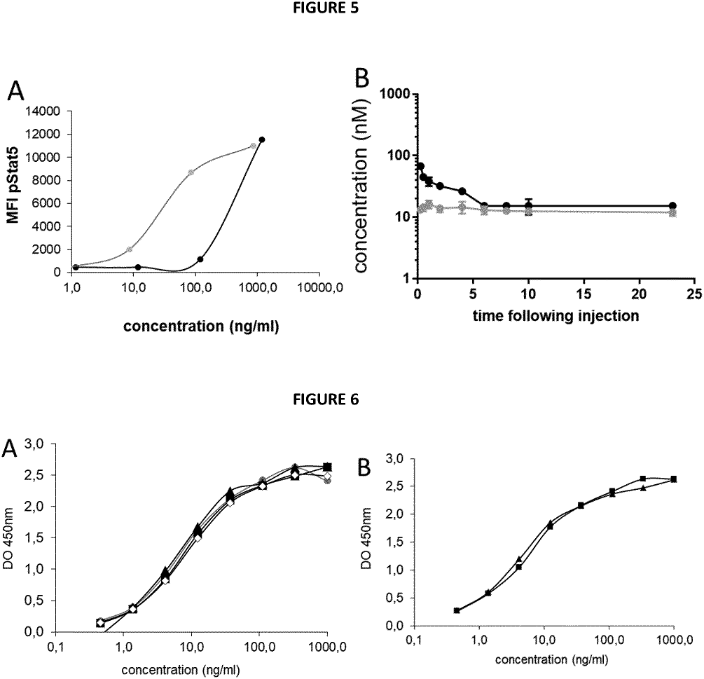

The addition of a cysteine at the C-terminal domain at the C-terminal domain of the IgG was also tested to create an additional disulfide bond and potentially restrict the flexibility of the IL-7 molecule. This mutant was named “C-IL-7”. FIG. 5 shows that the addition of a disulfide bounds in the IgG structure decreases pSTAT5 activity of the IL-7 compared to the anti PD-1 IL7 WT bifunctional molecule (FIG. 5A) and increases Cmax (5-fold) in the pharmacokinetics assay in vivo (FIG. 5B).

### Example 3: Anti PD-1 IL-7 Mutants Constructed With an IgG1N298A Isotype has a Better Binding to IL-7R, a Higher pSTAT5 Signaling and a Good Pharmacokinetics Profile In Vivo

Different isotypes of the anti PD-1 IL-7 bifunctional molecules were tested with IgG4m (S228P) or IgG1m (N298A or N297A depending on the numbering method). IgG4 isotype comprises the S228P mutation to prevent Fab arm-exchange in vivo and the IgG1 isotype comprises the N298A mutation that abrogates IgG1 isotype binding to FcyR receptors that may reduce the non-specific binding of the immunocytokine (mutant named “IgG4m” or “IgG1N298A”). Then, Anti PD-1 IL-7 bifunctional molecule was constructed with 2 different isotypes, IgG1 mutated in N297A (called IgG1m) isotype versus the IgG4 S288P isotype (called IgG4m) to determine whether the isotype structure modify the biological activity of IL-7 and its pharmacokinetics profile.

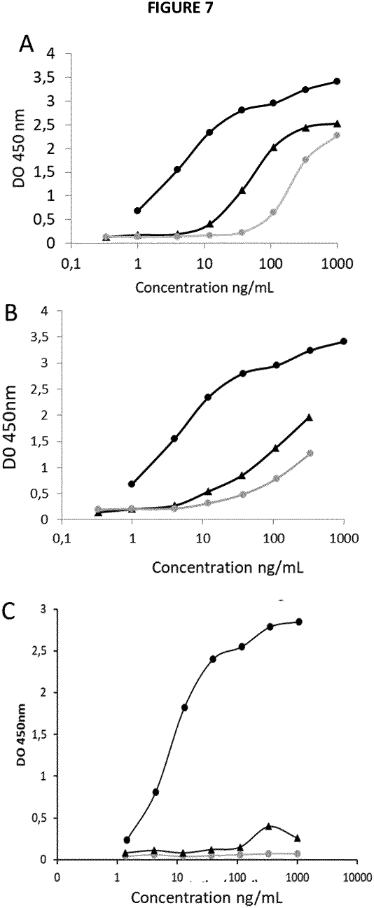

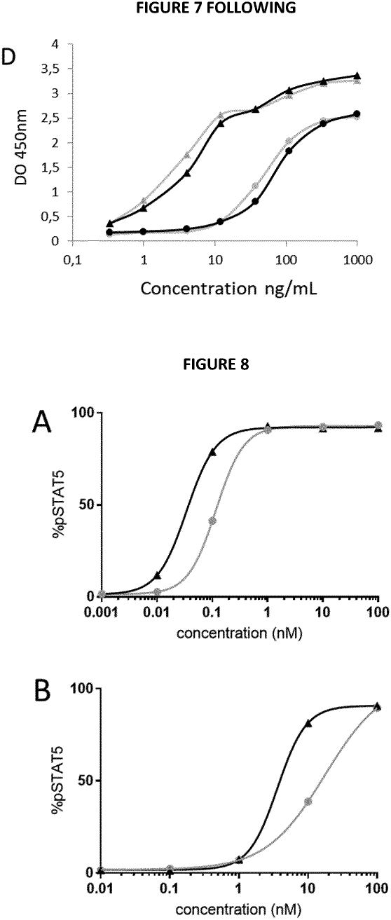

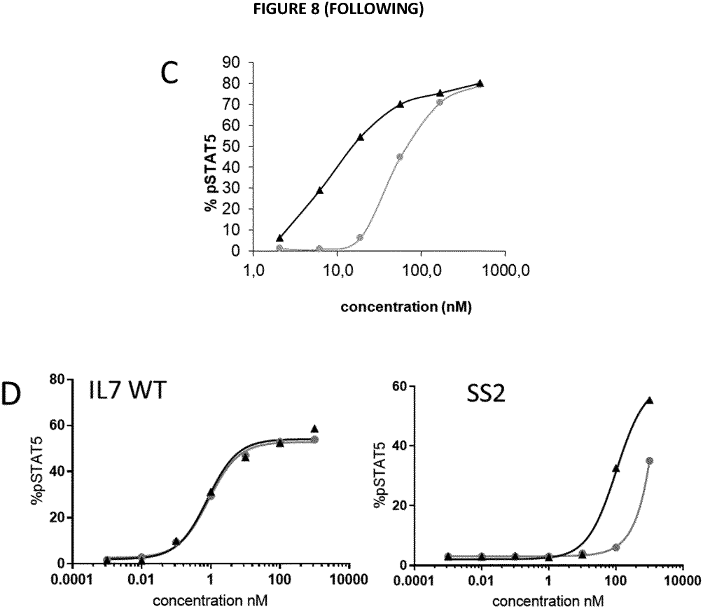

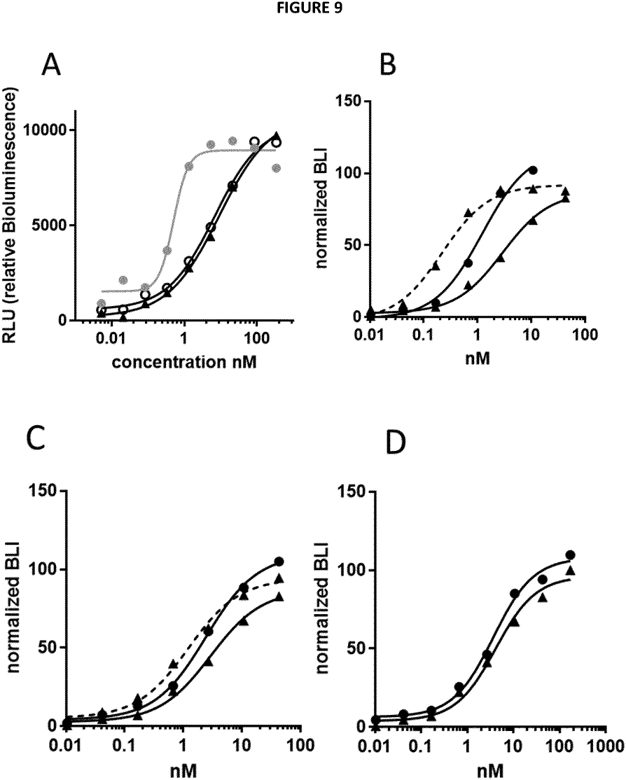

FIGS. 6A and 6B demonstrate that the anti PD-1 IL7 bifunctional molecules constructed with the IgG4m or IgG1m isotype have the same binding properties to PD-1 receptor, showing that the isotype does not modify the conformation of the VH and VL and the affinity of the anti PD-1 antibody for PD-1. However, the inventors observed that the IgG1m isotype unexpectedly improves the binding of the IL-7 D74, SS2 and slightly SS3 on CD127 (FIGS. 7A, B, C and D ) and pSTAT5 activation on human PBMCs (FIGS. 8A, B and C). This increase in pSTAT5 signalling was confirmed for the SS2 mutant on another T cell line (Jurkat cells expressing PD-1 and CD127, see FIG. 8D), but in a surprising manner, the IgG1m isotype does not modify pSTAT5 activity of the anti PD-1 IL-7 WT bifunctional molecule, suggesting that the IgG1m isotype only improves the activity of the IL-7 mutants.To determine the capacity of bifunctional molecule comprising an anti-PD1 antibody and an IL7 mutant to reactivate TCR mediated signaling, a NFAT Bioassay was performed. Results presented FIG. 9A show that the bifunctional molecule is better than an anti-PD1 or an anti-PD1+rIL7 (as separate compounds) to activate TCR mediated signaling (NFAT), demonstrating a synergistic effect of the bifunctional molecule on PD1+ T cells. The inventors next assessed the synergistic capacity of the bifunctional molecule comprising an anti PD-1 antibody and an IL-7 mutant (with mutation D74E, W142H or SS2) constructed with an IgG4m versus IgG1m isotype (FIGS. 9B, C, D). All the mutants tested conserve a synergistic effect on activating NFAT signaling with a level of activation correlated with their capacity to activate pSTAT5 signaling, in particular for bifunctional molecule with IL-7 D74E with IgG4m.

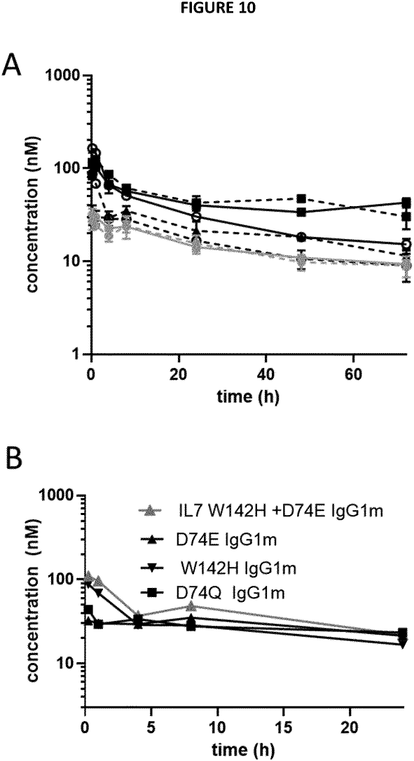

Pharmacokinetics study in mice demonstrate that IgG1 isotype does not modify the drug exposure for the IL7WT and SS3 molecule and a minimal impact on W142H molecule (FIG. 10A). Altogether these data show that an optimized isotype (IgG1m) is sufficient to enhance biological activity of the mutants while conserving a good pharmacokinetics of the product in vivo. With the IgG1m isotype, other IL-7 mutants were tested: D74N, D74Q and combination of D74E+ W142H mutation. No differences with the anti PD-1 IL-7 D74E mutant were observed on pSTAT5 activation (FIG. 9B) and pharmacokinetics (FIG. 10B).

The inventors particularly tested anti-PD-1 bifunctional molecule comprising IL-7 D74 mutants with different amino acid substitution D74E, D74Q and D74N. These constructions comprise an GGGGS linker and a IgG1N298A isotype. As detailed in the Table 6, all constructions have similar efficacy to bind PD-1 but the binding to the double PD-⅟CD127 is decreased with the D74Q and D74N mutant compared to D74E mutant suggesting that the substitution Q and N slightly attenuates the affinity of the mutant to CD127 receptor.

TABLE 6

ED50 determination of PD-1 and CD127 binding of the D74E, D74Q and D74N mutants. ED50 (ng/mL) refers to the concentration required to reach 50% of the binding to PD-1 and CD127 receptor binding measured by ELISA. PD-1 binding was measured by immobilization of the human PD-1 receptor and PD-⅟CD127 double binding was measured by immobilization of PD-1 and revelation with CD127 receptor as detailed in the material and method. All constructions tested comprise an GGGGS linker and an IgG1 N298A isotype

| Samples | Binding PD-1 (ED50 (ng/mL) | Double Binding PD-⅟CD127 (ED50 (ng/mL) |
|---|---|---|
| IgG1m G4S D74E | 6.4 | 4.9 |
| IgG1m G4S D74Q | 5.4 | 15.7 |
| IgG1m G4S D74N | 5.8 | 8.5 |

The double mutant D74E + W142H displayed similar profile compared to W124H IgG1 and the D74Q displayed a similar profile compared to D74E mutant. The inventors also constructed bifunctional molecules with IgG1m isotype + YTE mutation (M252Y/S254T/T256E). This mutation has been described to increase half life of antibody by increasing the binding to FcRn receptors. As shown on FIG. 7D, the YTE mutation does not modify the pSTAT5 signaling of the bifunctional molecule comprising the D74 or the W142H mutant.

### Example 4: The Mutation K444A Into the C-Terminal Lysine Residue Does Not Affect Pharmacokinetics In Vivo

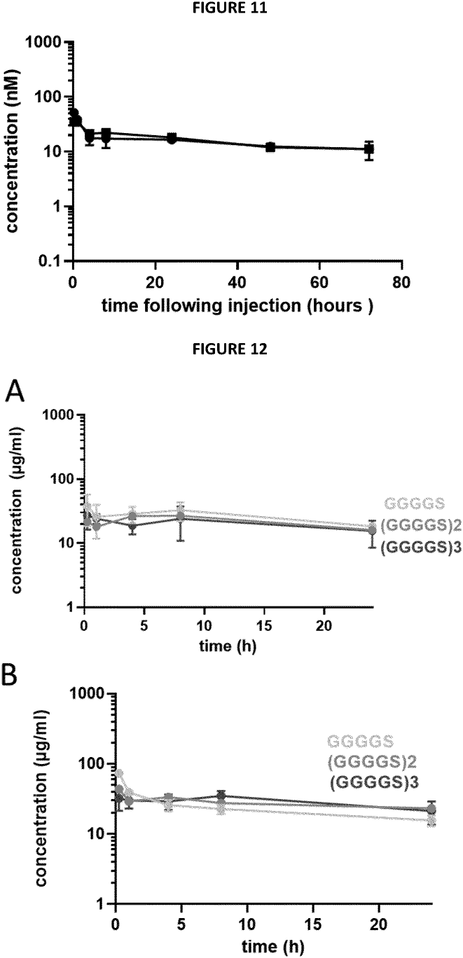

All subclass of Human IgG carries a C-terminal lysine residue of the antibody heavy chain (K444) that can be cleaved off in circulation. This cleavage in the blood may potentially compromises the bioactivity of the Immunocytokine by releasing the linked IL-7 to IgG. To circumvent this issue, K444 amino acid in the IgG domain was substituted by an alanine to reduce proteolytic cleavage, a mutation commonly used for antibodies. As shown in the FIG. 11, similar curve was obtained between IgG WT IL-7 versus IgG K444A IL-7 suggesting that the mutation does not affect the pharmacokinetic profile of the drug.

### Example 5: Linker Between IgG Antibody Does Not Modify Pharmacokinetics In Vivo but Improves Activation of pSTAT5 Signaling

Different linkers between IgG Fc domain and IL-7m were tested to modify flexibility. Several conditions were tested (e.g. no-linker, GGGGS, GGGGSGGGS, GGGGSGGGGS, GGGGSGGGGSGGGGS).

For the example 1 and 2, a linker (G4S)3 between the C-terminal domain of the Fc and the N-terminal domain of the IL-7 was used for the IgG4m-IL7 and IgG1m-IL-7 constructions, respectively. This linker allowed high flexibility and improvement of IL7 activation signal. To reduce affinity of IL7 to CD127 and improve the pharmacokinetics, different constructions were tested with varying the length of the linker (no linker, G4S, (G4S)2 or (G4S)3). For comparison, IgG1m or IgG4m Fc IL-7 WT was also generated with various linkers.

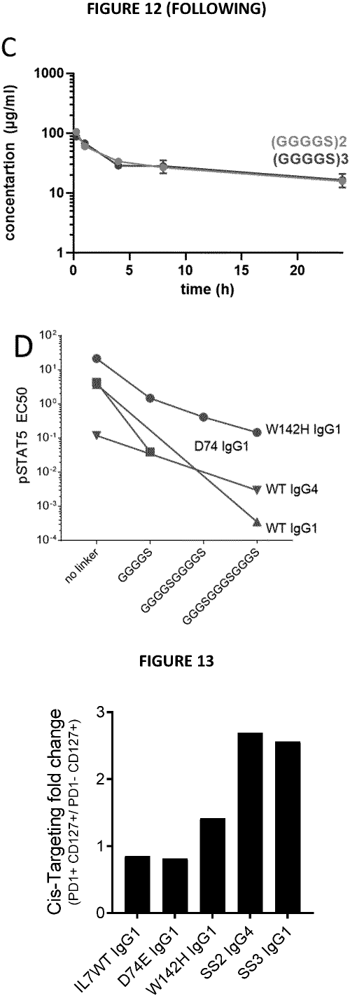

Pharmacokinetics study demonstrate that the length of the linker has no impact on the distribution, absorption and elimination of the product for the construction tested: Anti PD-1 IL7 WT (FIG. 12A), anti PD-1 IL-7 D74 (FIG. 12B) and anti PD-1 IL-7 W142H (FIG. 12C). However, the length of linker influences the activation of pStat5 as shown in FIG. 12D. Indeed, Anti PD-1 IL7 constructed with a linker (G4S)3 are more potent in activating pSTAT5 signaling compared to anti PD-1 IL-7 constructed with (G4S)2 or G4S3 linker and even more potent compared to anti PD-1 IL-7 constructed without linker. These data underscore the use of a (G4S)3 linker to allow flexibility of the IL-7 without compromising the pharmacokinetics of the drug in vivo.

### Example 6: The Anti PD-1 IL-7 Mutants Allow Preferential Binding on PD-1+ CD127+ Cells Over PD-1-CD127+ Cells

Next, the inventors assessed the capacity of the anti PD-1 IL-7 bifunctional molecule to target PD-1+ T cells. Jurkat cells expressing CD127+ or co-expressing CD127+ and PD-1+ were stained with 45 nM of the following bifunctional molecules: anti PD-1 IL-7 WT, D74, W142H, SS2 and SS3. The binding was detected with an anti IgG-PE (Biolegend, clone HP6017) and analyzed by flow cytometry.

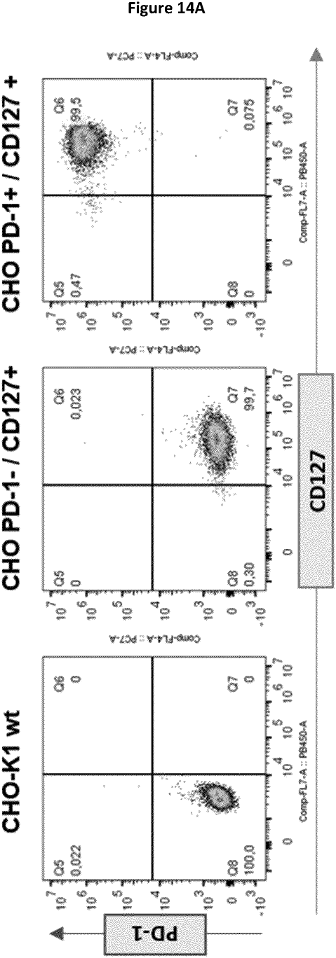

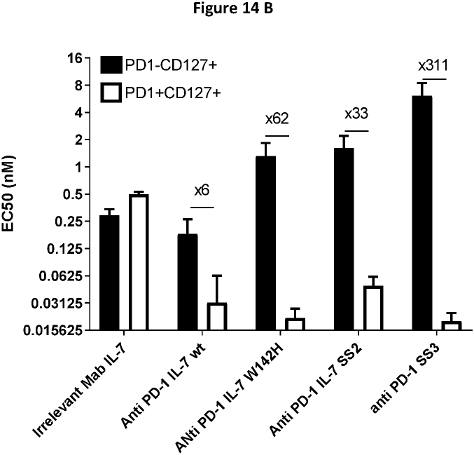

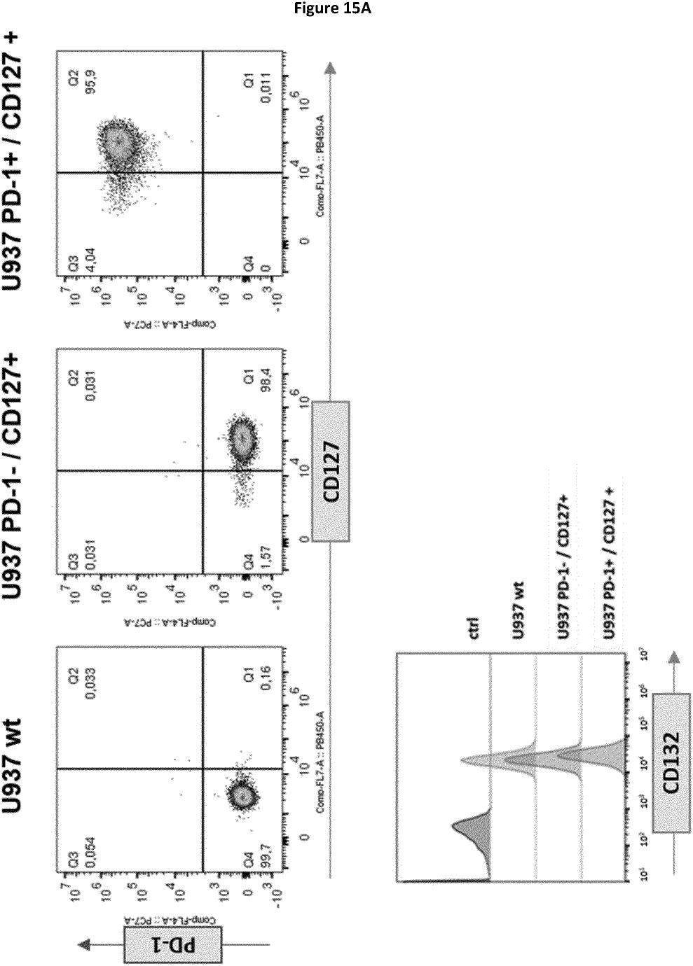

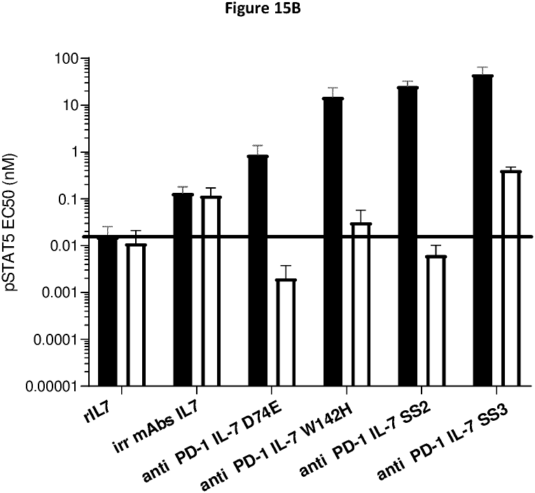

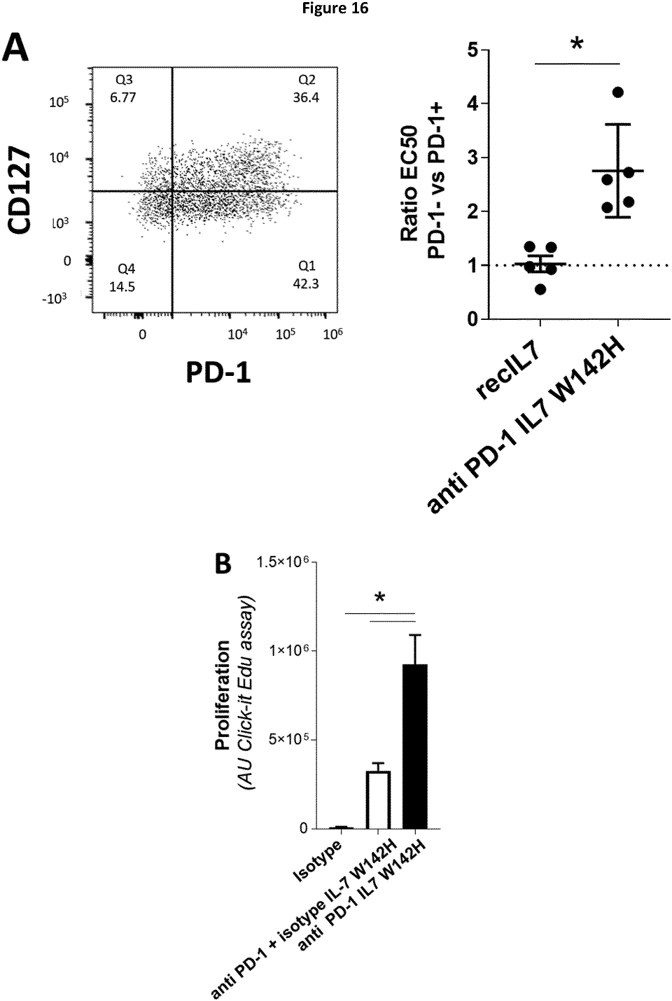

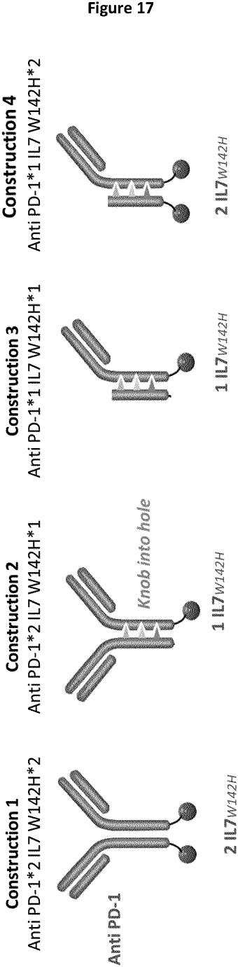

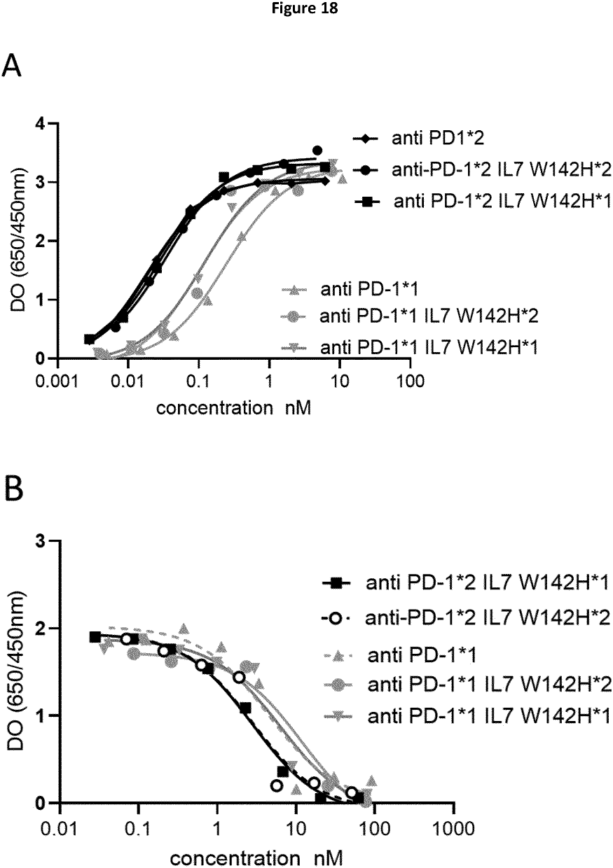

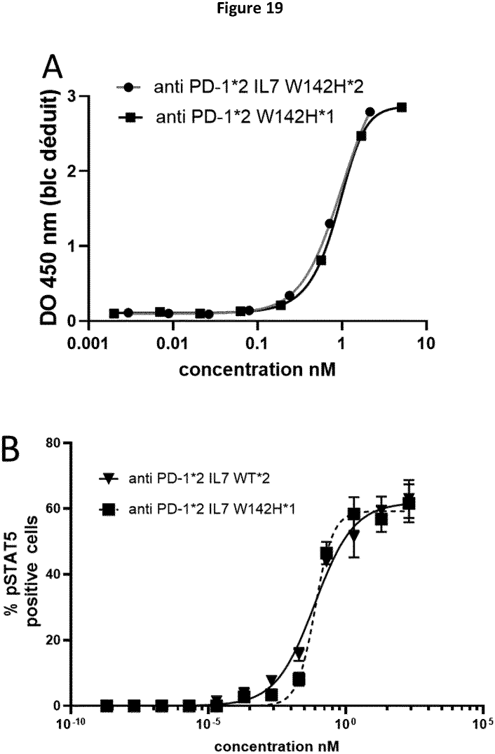

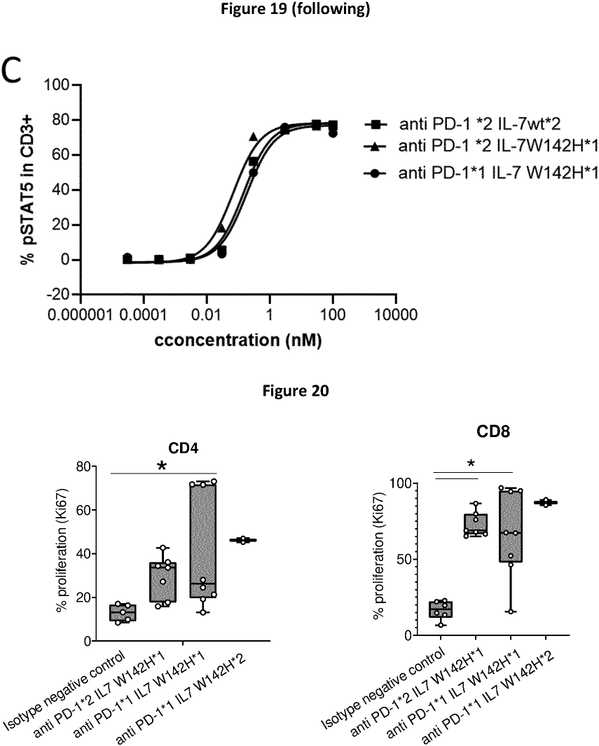

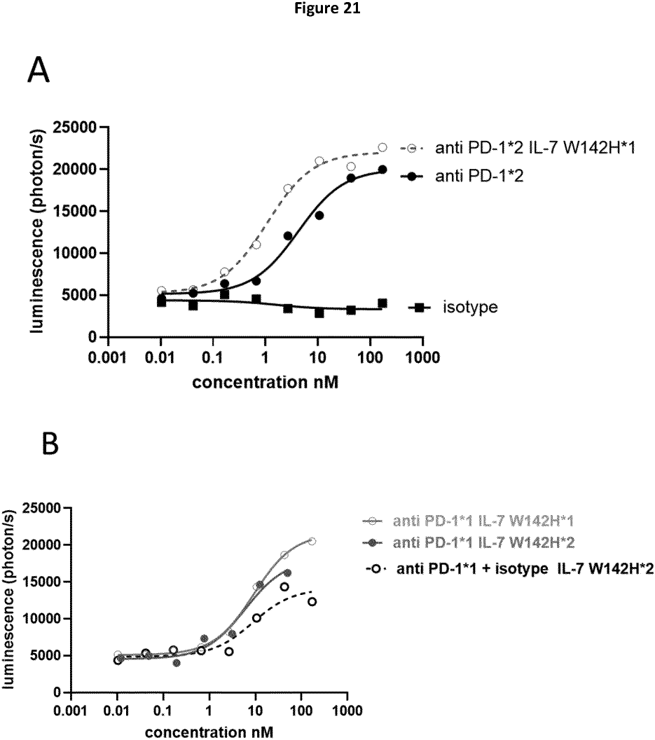

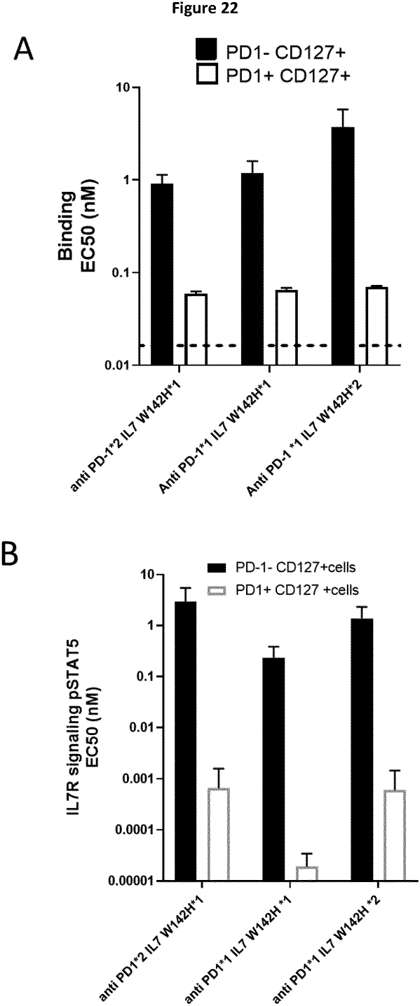

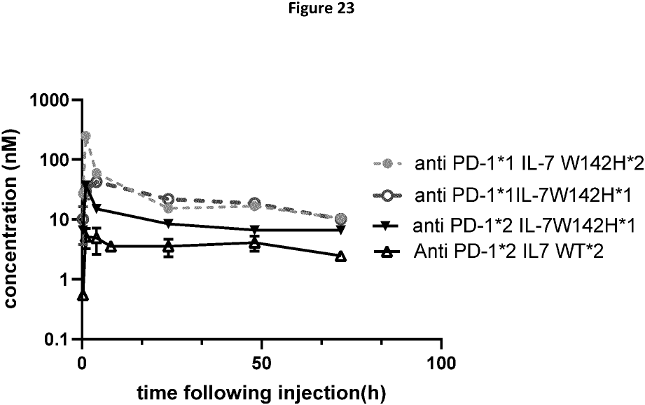

Results: FIG. 13 shows that anti PD-1 IL-7 WT and D74 mutant bind with similar efficacy to PD-1+/CD127+ cells versus PD-1-/CD127+ cells, whereas anti PD-1 IL-7 mutant SS2, SS3 binds with 2 to 3-fold higher efficacy to PD-1+/CD127+ cells versus PD-1-/CD127+ cells. The anti PD-1 IL-7 W142H bifunctional molecule shows an intermediate effect and binds with 1,4-fold higher efficacy to PD-1+/CD127+ cells.

To confirm the specific targeting of the anti PD-1 IL-7 mutant to PD-1+ T cells in heterogenous cellular model, the inventors next mixed PD-1(+) cells and PD-1(-) cells and analyzed their binding on each cell subset. In this assay, CHO cells co-expressing human CD127+ and human PD-1+ cells were co-cultivated at ratio 1:1 with CHO expressing human CD127+ receptor only (FIG. 14A ) then stained with escalating doses of bifunctional anti PD-1 IL-7 mutant D74E, W142H, SS2 and SS3 molecules, anti PD-1 alone or irrelevant isotype IL-7 antibody. The binding was revealed with an anti IgG-PE (Biolegend, clone HP6017) and analyzed by flow cytometry. EC50 binding (nM) was determined for each construction and each PD-1(+) and PD-1(-) cell population (FIG. 14B). Irrelevant isotype IL-7 control was used as negative control demonstrate an equal binding to PD-1(+) versus PD-1(-) cells. Although all bifunctional anti PD-1 IL-7 molecules preferentially bind to PD-1(+) cells over PD-1(-) cells in this co-culture assay, the inventors observed that IL-7 mutation improves the selective cis-binding of the molecule on PD-1+ cells. As shown in FIG. 14B, the anti PD-1 IL-7 W142H, SS2 and SS3 mutants demonstrated a strongly attenuated binding on PD-1(-)CD127(+) cells compared to anti-PD-1 IL-7 wild type, while the anti-PD-1 IL-7 mutants retained a potent binding (EC50^{~}300 pM) on PD-1(+)CD127(+)cells similar to the anti-PD-1 IL-7 wild type. In particular, the anti PD-1 IL-7 W142H and SS3 mutant showed the highest selective activity with respectively 62- and 311-fold difference binding between PD-1(+) cells versus PD-1(-) cells.

Altogether, these data show that the Il-7 mutation not only allows a better pharmacokinetics of the drug, but also allows the preferential binding of IL-7 on PD-1+ cells, i.e. targeting of the drug on the same cell. This aspect has an interest for the biological activity of the drug in vivo, as the anti PD-1 IL-7 will concentrate the IL-7 on PD-1+CD127+ exhausted T cells into the tumor microenvironment over CD127+ naïve T cells.

### Example 7: The Anti PD-1 IL-7 Mutant Bifunctional Molecule Preferentially Activates IL7R on PD-1+ Cells and Synergistically Promotes Proliferation of Human Activated T Cells

The IL-7R signaling activation (pSTAT5) was also tested in a coculture model of mixed U937 PD-1(+)CD127(+) and PD-1(-)CD127(+) cells. U937 cells also expressed the endogenous CD132 receptor required to transduce IL-7R signaling (FIG. 15A). The pSTAT5 signaling data demonstrate that PD-1 IL-7 mutants W142H, SS2 and SS3 have much higher selective activity in PD-1(+) cells over PD-1(-) cells. A 10 to 50-fold decreased activity is observed in PD-1(-) cells with the anti-PD-1 bifunctional molecule comprising anti IL7 mutants versus the anti PD-1 bifunctional molecule comprising IL-7 wild type (FIG. 15B). While a very low pSTAT5 activity was induced in PD-1(-) cells, a restored activity of the anti PD-1 bifunctional molecule comprising IL-7 mutants was obtained in PD-1(+) cells to similar extent to recombinant IL-7 wild type cytokine with an EC50 pSTAT5 activity close to 10pM. In particular, the W142H mutant has more than 450-fold more binding/activity in PD1+ cells as compared to PD-1- cells.

As the anti-PD-1 IL-7 bifunctional molecule was designed very advantageously and in particular to target PD-1(+)CD127(+) exhausted T cells, the inventors next analyzed the capacity of the anti PD-1 IL-7 W142H bifunctional molecule to preferentially activate pSTAT5 signaling and proliferation into primary human exhausted T cells. To generate PD-1(+)CD127(+) exhausted T cells, human peripheral blood T cells were subjected to repeated stimulation in vitro (αCD3/ αCD28) to mimic the chronic antigen stimulation occurring into the tumor microenvironment.

To assess the targeting effect of the bifunctional anti PD-1 IL-7 molecule to PD-1(+) T cells, exhausted T cells were incubated with a high concentration of anti PD-1 competitive antibody in order to block the binding of the anti PD-1 portion of the anti PD-1 IL-7 bifunctional molecule. Following incubation, exhausted T cells were treated with anti PD-1 IL-7 W142H bifunctional molecule or recombinant IL-7 wild type cytokine. pSTAT5 activation was then quantified by flow cytometry. The pSTAT5 activation ratio (EC50) between the two conditions (PD-1 blocking versus non-blocking isotype) was calculated and reported in FIG. 16A. Non-targeted IL-7 recombinant cytokine was used in this assay as negative control, and a ratio 1 was obtained showing a similar activity of the non-targeted IL-7 in PD-1(+) and PD-1(-) T cells. A significant differential activity was obtained after treatment with the anti PD-1 IL-7 W142H molecule (2 to 4-fold lower activity), suggesting that the molecule allows a preferentially cis-activation of the IL-7R signaling into PD-1(+) exhausted primary T cells over PD-1(-) exhausted T cells.

In addition, the inventors demonstrated that the specific cis-targeting of the anti PD-1 IL-7 W142H allow a synergistic proliferation of the exhausted T cells in vitro, while the combination of two separated agents (anti PD-1 antibody + isotype IL-7 W142H) induced significantly lower proliferation stimulation of exhausted T cells (FIG. 16B). Altogether these data confirm the advantage of the bifunctional molecule comprising a mutated IL-7 W142H molecule and an anti PD-1 antibody to selectively and synergistically cis-activate PD-1(+) CD127(+) exhausted T cells.

### Example 8: Anti PD-1 IL-7 Molecules With One IL-7 W142H Cytokine and One or 2 Anti PD-1 Arms Demonstrated a High Efficacy to Promote Cis Activity Into PD-1+ IL-7R+ Cells and to Stimulate IL-7R T Cell Proliferation In Vivo and a Synergistic Capacity to Reactivate TCR Signaling

The inventors next designed and compared the biological activity of multiple structures of bifunctional molecules comprising one or two anti PD-1 binding domains and one or two IL7 W142H mutants as described in FIG. 17.

Construction 1 comprises two anti PD-1 antigen binding domains and two IL-7 W142H variants (construction 1 is also called anti PD-1*2 IL-7 W142H*2), this molecule corresponds to the construction tested in the example 1 to 7. This molecule is also called BICKI-IL-7 W142H. In the examples, a control molecule called BICKI-IL-7 WT corresponds to construction 1 but with wild type IL-7.

Construction 2 comprises two anti PD-1 antigen binding domains and a single IL-7 W142H variant (construction 2 is also called anti PD-1*2 IL-7 W142H*1).

Construction 3 comprises a single anti PD-1 antigen binding domain and a single IL-7 W142H variant (construction 3 is also called anti PD-1*1 IL-7 W142H*1). A control construction called anti-PD-1*1 is similar than construction 3 but devoid of IL-7 variant.

Construction 4 comprises a single anti PD-1 antigen binding domain and two IL-7 W142H variants (construction 4 is also called anti PD-1*1 IL- W142H*2).

Constructions 2, 3 and 4 were engineered with an IgG1 N298A isotype and amino acid sequences were mutated in the Fc portion in order to create a knob on the CH2 and CH3 of the Heavy chains A and a hole on the CH2 and CH3 of the Heavy chains B.

All anti PD-1 IL7 constructions possess a high affinity to PD-1 receptor as demonstrated by ELISA assay (FIG. 18A and Table 7). Anti PD-1 IL-7 molecules having 2 anti PD-1 arms (anti-PD-1*2) have the same binding efficacy (equal EC50) compared to anti PD-1*2 without IL-7. Similarly, anti PD-1 IL-7 molecules having 1 anti PD-1 arm (Anti PD-1*1 IL7 W142H*1 and anti PD-1*1 IL7 W142H*2) demonstrated the same binding efficacy compared to the anti PD-1*1 without IL-7, with an EC50 equal to 86 and 111 nM for anti PD-1 IL7 versus 238 nM for the anti PD-1. These data show that fusion of IL-7 does not seem to interfere with the PD-1 binding regardless of the construction tested.

TABLE 7

ED50 determination from FIG. 18A refers to the concentration required to reach 50% of the PD1 binding signal as measured by ELISA for each anti PD-1 IL-7 molecule

| Samples | EC50 (nM) |
|---|---|
| anti PD-1*2 | 0.021 |
| anti-PD-1*2 IL7 W142H*1 | 0.026 |
| anti-PD-1*2 IL7 W142H*2 | 0.034 |
| anti-PD-1*1 | 0.238 |
| anti-PD-1*1 IL7 W142H*1 | 0.111 |
| anti-PD-1*1 IL7 W142H*2 | 0.086 |

Moreover, PD-L1/PD-1 antagonist bioassay (FIG. 18B) demonstrates that anti PD-1 IL7 molecules having 1 or 2 anti PD-1 arms display high efficiency to block the binding of PD-L1 to the PD-1 receptor. Although one arm of anti PD-1 was removed from the constructions 3 and 4, all the anti PD-1*1 IL7 construction demonstrates high antagonist properties. Only a 2.5-fold decreased activity compared to anti PD-1*2 IL7 constructions was calculated with EC50 (Table 8) for the constructions 3 and 4.

TABLE 8

ED50 determination from FIG. 18B refers to the concentration required to reach 50% of the PD1/PDL1 antagonist activity as measured by ELISA for each anti PD-1 IL-7 molecule

| Samples | EC50 (nM) |
|---|---|
| anti PD-1*2 IL7 W142H*1 | 2.168 |
| anti PD-1*2 IL7 W142H*2 | 2.792 |
| anti PD-1*1 | 5.014 |
| anti PD-1*1 IL7 W142H *1 | 5.839 |
| anti PD-1*1 IL7 W142H *2 | 7.235 |

The inventors next assessed the affinity of the different constructions to CD127 receptor using Biacore assay and ELISA assay. Since one IL-7 molecule was removed from construction 2 and 3, a lower binding capacity to CD127 receptor and a lower pSTAT5 activation was expected for these molecules in comparison to the IL-7 heterodimeric constructions. However, the inventors observed that the anti PD-1*2 IL-7 W142H*1 molecule has similar affinity to CD127 receptor compared to the anti PD-1*2 IL-7 W142H*2 (BICKI-IL-7 W142H) and as lower affinity compared to the anti PD-1 IL7 bifunctional molecules comprising IL-7 wild type form (FIG. 19A and Table 9). Surprisingly, the anti PD-1*2 IL7 W142H *1 molecules demonstrate a high pSTAT5 activity similar to the PD-1 IL7 bifunctional molecules comprising IL-7 wild type form (FIG. 19B). Based on these observations, it could be hypothesized that the monomeric form of IL-7 combined with W142H IL-7 mutation allows an optimal conformation of the IL-7 molecule to promote IL-7 signaling into human T cells. Even with only one IL7, the molecule with W142H IL-7 mutation has an activation effect (pSTAT5) as good as a molecule with IL7 wt with two cytokines. This result is surprising in the context of an IL-7 variant having a lower affinity for its receptor than the wild type IL-7.

Similar conclusions were drawn with anti PD-1 IL7 molecules constructed with one anti PD-1 arm fused to one IL-7 W142H mutant. A similar and comparable high pSTAT5 activity was obtained with the anti PD-1*2 IL-7WT *2, the anti PD-1*2 IL-7 W142H*1 and the anti PD-1*1 IL-7 W142H*1 constructions (FIG. 19C).

TABLE 9

Binding of anti PD1 IL7 wildtype or anti PD1 IL7 W142H mutant constructed with 1 or 2 IL7. CD127 was immobilized to the sensor chip and anti PD-1 IL-7 bifunctional molecules were added at escalating doses to measure affinity

| | |
|---|---|
| | KD CD127 (M) |
| anti PD-1*2 IL7 wild type*2 | 8.7 E-10 |
| anti PD-1*2 IL7 W142H*2 | 3.73 E-8 |
| anti PD-1*2 IL7 W142H*1 | 4.52 E-8 |

In vivo experiments were performed to determine the efficacy of the different anti PD-1 IL-7 constructions. One dose of anti PD-1 IL-7 molecules was injected into mice at equivalent molarity concentration (34 nM/kg). On Day 4 following treatment, CD4 and CD8 T cell proliferation was quantified by flow cytometry using Ki67 marker. FIG. 20 shows that the anti PD-1 IL7 molecules having a single W142H mutant (anti PD-1*2 IL-7 W142H*1 and anti PD-1*1 IL-7 W142H *1) or having a single PD-1 valency and two IL7 W142H cytokines (anti PD-1*1IL7W142H*2) display high efficiency in promoting CD8 and to a lesser extent CD4 T cells proliferation.

To determine the capacity of bifunctional molecules comprising an anti-PD1 antibody (one or 2 valences) and a one or two IL7 mutant cytokines to reactivate TCR mediated signaling, a NFAT Bioassay was performed. FIG. 21A shows that the bifunctional molecule constructed with 2 anti PD-1 arms and one IL-7 cytokine enhances the activation of NFAT compared to the anti PD-1 antibody alone, demonstrating that the synergistic activity of the drug to strengthen the TCR mediated signaling is conserved with an anti PD-1 IL-7 bifunctional molecule constructed with only one IL-7 cytokine. As seen in FIG. 9A, there was no such synergy when cells were treated with the combination of anti-PD1 plus IL7.

In addition, the inventors next assessed activity of the anti PD-1 IL-7 molecule designed with only one anti PD-1 valency (Anti PD-1*1) and demonstrate that the anti PD-1*1 IL-7 W142H constructions (Anti PD-1*1 IL7 W142H *1 and *2) retain their synergistic activity, whereas the combination PD-1*1 + isotype IL-7 W142H*2 treatment shows less efficacy in stimulating TCR signaling (NFAT activation) (FIG. 21B).

Finally, the specific cis-targeting and cis-activity of the different anti PD-1 IL-7 constructions were analyzed in a co-culture assay. U937 PD-1+ CD127+ cells were mixed with PD-1- CD127+ cells (ratio 1:1), then incubated with the different constructions at escalating doses. The binding and the IL-7R signaling (pSTAT5) was quantified by flow cytometry. EC50 (nM) of the binding and the pSTAT5 activation was determined for each construction and for each PD-1 + and PD-1- cell population (FIGS. 22A and B). The inventors validated that a diversity of anti PD-1 IL-7 mutated molecules (anti PD-1*2 IL7 W142H*1, anti PD-1*1 IL7 W142H*1 anti PD-1*1 IL7 W142H*2) substantially preferentially bind IL-7R into PD-1+ cells, with a huge activation of IL7R signaling pSTAT5 into PD-1+ cells for different and representative structures.

### Example 9: Anti PD-1 IL-7 Molecules Constructed With 1 or 2 Arms of Anti PD-1 and 1 or 2 IL7 W142H Cytokines Have a Good Pharmacokinetic Profile In Vivo

Pharmacokinetics study of the anti PD-1 IL-7 bifunctional molecules constructions 2, 3 and 4 such as described in FIG. 17 was assessed. Humanized PD1 KI Mice were intraperitoneally injected with one dose of anti PD-1 IL-7 molecules (34.4 nM/kg). Plasma drug concentration was analyzed by ELISA specific for human IgG (FIG. 23). Area under the curve was also calculated (see Table 10) and represents the total drug exposure across time for each construction. The anti PD-1*2IL-7 W142H*1, anti PD-1* 1 IL-7 W142H*1 and anti PD-1*1 IL-7 W142H*2 constructions demonstrated a very advantageously enhanced PK profile compared to the anti PD-1*2 IL7WT*1. A Cmax 2.8 to 19 fold higher was observed compared to the anti PD-1*1 IL7WT *2. Importantly, a high drug concentration (11-15 nM) which corresponds to a satisfying PK value in vivo, is maintained for at least 96 hours with the anti PD-1*1 IL7 W142H*1 anti PD-1*1 IL7 W142H*2 molecules whereas only 2 nM of anti PD-1*2 IL7WT*2 molecule is detected in the plasma. A residual drug concentration with the anti PD-1*2 IL-7 W142H*1 is 2,5-fold higher than the anti PD-1*2 IL7WT*2 concentration. Plasma drug exposure is often correlated with efficacy in vivo. Here, the inventors demonstrate that all anti PD-1 IL-7 W142H molecules constructed with one arm of anti PD-1 allows a long-term drug exposure following a single injection, suggesting that these constructions will induce a higher biological activity in vivo.

TABLE 10

Area under the curve determination from FIG. 23 . AUC was calculated from 0 to 96 hours following intraperitoneal injection of one dose of anti PD-1 IL-7 (34 nM/kg)

| | AUC | Cmax (nM) |
|---|---|---|
| anti PD1*1 IL7W142H*1 | 1597 | 42.4 |
| antiPD-1*1 IL7W142H*2 | 2024 | 248.6 |

It is also mentioned that, even if certain molecules PD-1*2 IL7WT*2 with IL7 wild type may have also a good PK (in particular for intravenous injection) as compared to IL7 W142H, the molecules with IL7 W142H have further better other properties: a better proliferation of T cells (CD4, CD8 as shown in FIG. 20) and a much better specific targeting of PD1+ cells vs PD1- cells (10 to 50-fold as explained for FIG. 15B).

As a whole, a plurality of constructs of bifunctional molecules with mutated IL7 (notably W142H) have been obtained with a very satisfying PK for effective pharmaceutical use (preferably at least 10 nM after 24 hours), and with further:
a substantial advantageous effect on LT proliferation,
a high performance in terms of LT activation through IL7R signaling pSTAT5 into PD-1+ cells, thanks to the synergistic surprising effect on T cells between the anti PD1 part of the bifunctional molecule and the IL7 part of the bifunctional molecule,
a high specific targeting of PD1+ T cells versus PD1- T cells (much higher than bifunctional molecules not having mutated IL7), and cis-activation of the IL-7R signaling into PD-1+ exhausted primary T cells over PD-1- T cells, which is a substantial advantage for tumor treatment; and
an effective antagonist effect of PD-⅟PD-L1 interaction (not only a binding to PD1).

### Material and Method

### ELISA Binding PD1

For activity ELISA assay, recombinant hPD1 (Sino Biologicals, Beijing, China; reference 10377-H08H) was immobilized on plastic at 0.5 µg/ml in carbonate buffer (pH9.2) and purified antibody were added to measure binding. After incubation and washing, peroxidase-labeled donkey anti-human IgG (Jackson Immunoresearch; USA; reference 709-035-149) was added and revealed by conventional methods.

### Affinity Measurement Using Biacore Method

Affinity assessment by Biacore of IgG fused to IL-7 on its heavy chains for CD127 (A) or CD132 (B). CD127 (Sinobiological, 10975-H03H-50) was immobilized onto a CM5 biochip at 20 µg/ml and the indicated protein were added at serial concentrations (0.35; 1.1; 3.3; 10; 30 nM). Affinity was analyzed using two state reaction models. To assess affinity of IL-7 to CD132, CD127 was immobilized on the CM5biochip and each IL-7 construction was injected at a concentration of 30 nM. The CD132 receptor (Sinobiological 10555-H08B) was added at different concentrations, e.g. 31.25, 52.5, 125, 250, 500 nM. A steady state affinity model was used for analysis.

### CD127 Binding ELISA

CD127 binding was assessed by a sandwich ELISA method. Recombinant proteins targeted by the antibody backbone were immobilized, then antibodies fused IL-7 preincubated with CD127 recombinant protein (Histidine tagged, Sino ref 10975-H08H) were incubated. Revelation was performed with a mixture of an anti-histidine antibody (MBL #D291-6) coupled to biotin and streptavidin coupled to Peroxidase (JI 016-030-084). Colorimetry was determined at 450 nm using TMB substrate.

### pSTAT5 Analysis

PBMCs isolated from peripheral blood of human healthy volunteers were incubated 15 minutes with recombinant IL-7, or IgG fused IL-7. To determine cis activity, U937 cells transduced with CD127+ PD-1+ were mixed with U937 cells transduced with CD127+ only. Cells were mixed at a ratio 1:1 and treated with recombinant IL-7, or the different IgG fused to IL-7 constructions described herein. Each cells subset was labeled with Cell proliferation dye (CPDe450 or CPDe670) prior to coculture. Cells were then fixed, permeabilized and stained with an AF647 labeled anti-pSTAT5 (clone 47/Stat5(pY694)). Data were obtained by calculating MFI pSTAT5 in CD3+ T cell population.

### Cellular Binding Analysis

To determine cis binding of the IgG fused to IL-7 molecules, U937 or CHO cells transduced with CD127+ PD-1+ were mixed with CHO or U937 transduced with CD127+ only. Cells were mixed at a ratio 1:1 and treated with the different IgG fused to IL-7 constructions described herein. Each cells subset was labeled with Cell proliferation dye (CPDe450 or CPDe670) prior to coculture. After 20 minutes of incubation, the binding of the different IgG fused molecules was detected with an anti IgG-PE antibody (Biolegend, clone HP6017) and analyzed by flow cytometry.

### Pharmacokinetics of the IgG Fused IL-7 In Vivo

To analyze the pharmacokinetics of the IL-7 immunocytokine, a single dose of the molecule was intra-orbitally or intraperitoneally injected to BalbcRJ mice (female 6-9 weeks) or C57bl6JrJ mice (female 6-9 weeks). Drug concentration in the plasma was determined by ELISA using an immobilized anti-human light chain antibody (clone NaM76-5F3) diluted serum containing IgG fused Il67. Detection was performed with a peroxidase-labeled donkey anti-human IgG (Jackson Immunoresearch; USA; reference 709-035-149) and revealed by conventional methods.

### T Cell Activation Assay Using Promega Cell-Based Bioassay

The capacity of anti-PD-1 antibodies restore T cell activation was tested using Promega PD-⅟PD-L1 kit (Reference J1250). Two cell lines are used (1) Effector T cells (Jurkat stably expressing PD-1, NFAT-induced luciferase) and (2) activating target cells (CHO K1 cells stably expressing PDL1 and surface protein designed to stimulate cognate TCRs in an antigen-independent manner. When cells are cocultured, PD-L1 /PD-1 interaction inhibits TCR mediated activation thereby blocking NFAT activation and luciferase activity. The addition of an anti- PD-1 antibody blocks the PD-1 mediated inhibitory signal leading to NFAT activation and luciferase synthesis and emission of bioluminescence signal. Experiment was performed as per as manufacturer recommendations. Serial dilutions of the PD-1 antibody were tested. Four hours following coculture of PD-L1+ target cells, PD-1 effector cells and anti PD-1 antibodies, BioGlo™ luciferin substrate was added to the wells and plates were read using Tecan™ luminometer.

### In Vivo Proliferation

A single dose of bifunctional molecules (34 nM/kg) was intraperitoneally injected to C57bl6JrJ mice (female 6-9 weeks) bearing a subcutaneous MC38 tumor. On Day 4 following treatment, Blood an MC38 tumor were collected and T cells were stained with an anti CD3, anti CD8, anti CD4 antibody and an anti ki67 antibody to quantify proliferation by flow cytometry.

### Antibodies and Bifunctional Molecules

The following antibodies and bifunctional molecules have been used in the different experiments disclosed herein: Pembrolizumab (Keytrudra, Merck) Nivolumab (Opdivo, Bristol-Myers Squibb), and the bifunctional molecules as disclosed herein comprising an anti-PD1 humanized antibody comprising a variable heavy chain (VH) as defined in SEQ ID NO: 24 and a variable light chain (VL) as defined in SEQ ID NO: 28 or an anti-PD1 chimeric antibody comprising an heavy chain as defined is SEQ ID NO: 71 and a light chain as defined in SEQ ID NO: 72.

Construction 1 comprises two anti PD-1 antigen binding domains and two IL-7 W142H variants (construction 1 is also called anti PD-1*2 IL-7 W142H*2). This molecule corresponds to the construction tested in the example 1 to 7. This molecule is also called BICKI-IL-7 W142H. In particular, construction 1 comprises a variable heavy chain (VH) as defined in SEQ ID NO: 24 and a variable light chain (VL) as defined in SEQ ID NO: 28 or an anti-PD1 chimeric antibody comprising a heavy chain as defined is SEQ ID NO: 71 and a light chain as defined in SEQ ID NO: 72. The molecule also comprises the IL7 variant such as described in SEQ ID NO: 5.

In the examples, a control molecule called BICKI-IL-7 WT corresponds to construction 1 but with wild type IL-7. It comprises a variable heavy chain (VH) as defined in SEQ ID NO: 24 and a variable light chain (VL) as defined in SEQ ID NO: 28. The molecule has an IgG4 S288P isotype.

Another control molecule is anti-PD1*2 (without any IL7). The molecule comprises a heavy chain as defined in SEQ ID NO: 79 and a light chain as defined in SEQ ID NO: 80.

Construction 2 comprises two anti PD-1 antigen binding domains and a single IL-7 W142H variant (construction 2 is also called anti PD-1*2 IL-7 W142H*1). In particular, construction 2 comprises a variable heavy chain (VH) as defined in SEQ ID NO: 24 and a variable light chain (VL) as defined in SEQ ID NO: 28. The molecule particularly comprises a heavy chain bound to IL-7 W142H as defined is SEQ ID NO: 83 (hole) or a heavy chain as defined is SEQ ID NO: 81 (knob) and a light chain as defined in SEQ ID NO: 80.

Construction 3 comprises a single anti PD-1 antigen binding domain and a single IL-7 W142H variant (construction 3 is also called anti PD-1*1 IL-7 W142H*1). In particular, construction 3 comprises a variable heavy chain (VH) as defined in SEQ ID NO: 24 and a variable light chain (VL) as defined in SEQ ID NO: 28. The molecule comprises a heavy chain bound to IL-7 W142H as defined is SEQ ID NO: 83, a Fc region as defined in SEQ ID NO: 75 and a light chain as defined in SEQ ID NO: 80.

A control construction called anti-PD-1*1 is similar than construction 3 but devoid of IL-7 variant. Such control comprises a variable heavy chain (VH) as defined in SEQ ID NO: 24 and a variable light chain (VL) as defined in SEQ ID NO: 28. The molecule comprises a heavy chain as defined is SEQ ID NO: 81, a Fc region as defined in SEQ ID NO: 75 and a light chain as defined in SEQ ID NO: 80.

Construction 4 comprises a single anti PD-1 antigen binding domain and two IL-7 W142H variants (construction 4 is also called anti PD-1*1 IL-W142H*2). In particular, construction 4 comprises a variable heavy chain (VH) as defined in SEQ ID NO: 24 and a variable light chain (VL) as defined in SEQ ID NO: 28. The molecule comprises a heavy chain bound to IL-7 W142H as defined is SEQ ID NO: 83, a Fc region bound to IL-7 W142H as defined in SEQ ID NO: 76 and a light chain as defined in SEQ ID NO: 80.

Constructions 2, 3 and 4 were engineered with an IgG1 N298A isotype and amino acid sequences were mutated in the Fc portion in order to create a knob on the CH2 and CH3 of the Heavy chains A and a hole on the CH2 and CH3 of the Heavy chains B. All anti PD-1 IL-7 and anti PD-1*1 constructions comprise an IgG1N298A mutated isotype excepted the anti PD-1*2 construction (lacking IL-7) and anti-PD-1*2 IL7wt*2 (BICKI-IL-7 WT) that were constructed with an IgG4 S288P isotype.

1-24. (canceled)

25. A bifunctional molecule comprising an interleukin 7 (IL-7) variant conjugated to a binding moiety, wherein:
the binding moiety binds to a target specifically expressed on immune cells surface,
the IL-7 variant presents at least 75% identity with a wild type human IL-7 (wth-IL-7) comprising SEQ ID NO: 1, wherein the variant comprises at least one amino acid mutation selected from the group consisting of (i) W142H, W142F or W142Y, (ii) C2S-C141S and C47S-C92S, C2S-C141S and C34S-C129S, or C47S-C92S and C34S-C129S, (iii) D74E, D74Q or D74N, iv) Q11E, Y12F, M17L, Q22E and/or K81R; or any combination thereof, the amino acid numbering being as shown in SEQ ID NO: 1, which i) reduces affinity of the IL-7 variant for IL-7 receptor (IL-7R) in comparison to the affinity of wth-IL-7 for IL-7R, and ii) improves pharmacokinetics of the bifunctional molecule comprising the IL-7 variant in comparison with a bifunctional molecule comprising wth-IL-7.

26. The molecule according to claim 25, wherein the IL-7 variant comprises an amino acid substitution selected from the group consisting of W142H, W142F and W142Y, the amino acid numbering being as shown in SEQ ID NO: 1.

27. The molecule according to claim 25, wherein the IL-7 variant comprises a group of amino acid substitutions selected from the group consisting of C2S-C141S and C47S-C92S, C2S-C141S and C34S-C129S, and C47S-C92S and C34S-C129S, the amino acid numbering being as shown in SEQ ID NO: 1.

28. The molecule according to claim 25, wherein the IL-7 variant comprises an amino acid substitution selected from the group consisting of D74E, D74Q and D74N, the amino acid numbering being as shown in SEQ ID NO: 1.

29. The molecule according to claim 25, wherein the IL-7 variant comprises SEQ ID NO: 2, 3, 4, 5, 6, 7, 8, 9, 10, 11, 12, 13, 14 or 15.

30. The molecule according to claim 25, wherein the binding moiety comprises a heavy chain constant domain or a Fc domain of a human IgG1, optionally with a substitution or a combination of substitutions selected from the group consisting of T250Q/M428L; M252Y/S254T/T256E + H433K/N434F; E233P/L234V/L235A/G236A + A327G/A330S/P331S; E333A; S239D/A330L/I332E; P257I/Q311; K326W/E333S; S239D/I332E/G236A; N297A; L234A/L235A; N297A + M252Y/S254T/T256E; and K322A and K444A.

31. The molecule according to claim 25, wherein the binding moiety comprises a heavy chain constant domain or a Fc domain of a human IgG4, optionally with a substitution or a combination of substitutions selected from the group consisting of S228P, L234A/L235A, S228P + M252Y/S254T/T256E.17 and K444A.

32. The molecule according to claim 25, wherein the immune cell is a T cell or an exhausted T cell.

33. The molecule according to claim 32, wherein the target is expressed by T cells and the binding moiety binds to a target selected from the group consisting of PD-1, CD28, CD80, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CXCR5, CD3, PDL2, CD4 and CD8.

34. The molecule according to claim 32, wherein the target is expressed by T exhausted cells and the binding moiety binds to a target selected from the group consisting of PD-1, CTLA-4, BTLA, TIGIT, LAG3 and TIM3.

35. The molecule according to claim 25, wherein the binding moiety is an antibody or an antigen fragment thereof, and the N-terminus of the IL-7 variant is fused to the C-terminus of a heavy or light chain constant domain of the antibody or antibody fragment thereof, optionally via a peptide linker.

36. The molecule according to claim 35, wherein the IL-7 variant is fused to the binding moiety by a peptide linker selected from the group consisting of GGGGS (SEQ ID NO: 68), GGGGSGGGS (SEQ ID NO: 67), GGGGSGGGGS (SEQ ID NO: 69) and GGGGSGGGGSGGGGS (SEQ ID NO: 70).

37. The molecule according to claim 25, wherein the molecule comprises a first monomer comprising an antigen-binding domain covalently linked via its C-terminal end to N-terminal end of a first heterodimeric Fc chain optionally via a peptide linker, said first heterodimeric Fc chain being covalently linked by the C-terminal end to the N-terminal end of the IL-7 variant, optionally via a peptide linker, and a second monomer comprising a complementary second heterodimeric Fc chain devoid of antigen-binding domain.

38. The molecule according to claim 37, wherein, in the second monomer, the complementary second heterodimeric Fc chain covalently linked to the IL-7 variant, optionally via a peptide linker.

39. The molecule according to claim 25, wherein the molecule comprises a first monomer comprising an antigen-binding domain covalently linked by C-terminal end to N-terminal end of a first heterodimeric Fc chain, optionally via a peptide linker, said first heterodimeric Fc chain being devoid of IL-7 variant, and a second monomer comprising a complementary second heterodimeric Fc chain devoid of antigen-binding domain, said second heterodimeric Fc chain being covalently to the IL-7 variant, optionally via a peptide linker.

40. The molecule according to claim 25, wherein the molecule comprises a first monomer comprising an antigen-binding domain covalently linked via its C-terminal end to N-terminal end of a first heterodimeric Fc chain optionally via a peptide linker, and a second monomer comprising an antigen-binding domain covalently linked via C-terminal end to N-terminal end of a complementary second heterodimeric Fc chain optionally via a peptide linker, wherein only one of heterodimeric Fc chains is covalently linked by the C-terminal end to the N-terminal end of the IL-7 variant.

41. The molecule according to claim 37, wherein the antigen-binding domain is a Fab domain, a Fab′, a single-chain variable fragment (scFV) or a single domain antibody (sdAb).

42. The molecule according to claim 37, wherein the antigen-binding domain comprises: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or SEQ ID NO: 65, a CDR2 of SEQ ID NO: 66 and a CDR3 of SEQ ID NO: 16.

43. The molecule according to claim 37, wherein the antigen-binding domain comprises:
(a) a heavy chain variable region (VH) comprising SEQ ID NO: 18, 19, 20, 21, 22, 23, 24 or 25;
(b) a light chain variable region (VL) comprising SEQ ID NO: 27 or SEQ ID NO: 28.

44. The molecule according to claim 43, wherein the antigen-binding domain comprises or consists essentially of a heavy chain variable region (VH) of SEQ ID NO: 24 and a light chain variable region (VL) of SEQ ID NO: 28.

45. An isolated nucleic acid sequence or a group of isolated nucleic acid molecules encoding the bifunctional molecule according to claim 25.

46. A host cell comprising the isolated nucleic acid according to claim 45.

47. A pharmaceutical composition comprising the bifunctional molecule according to claim 25 and a pharmaceutically acceptable carrier.

48. A method of treating cancer or an infectious disease comprising the administration of a molecule according to claim 25, or a pharmaceutical composition comprising said molecule, to a subject having cancer or an infectious disease.
